SEQUENCE LISTING (81 sequences)

SEQ ID NO: 1 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 2 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 3 - DNA, 1375 bp, Zea mays
agttcatcactaatcacacttattgtgccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaactacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgcgctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaacccgtcgaactgccgcaggttcctggtgctgtccatcgggacggggtcgacgtccgagcagggcctctacacggcgcggcagtgctcccggtggggcatctgccggtggctccgcaacaacggcatggcccccatcatcgacatcttcatggcggccagctcggacctggtggacatccacgtcgccgcgatgttccagtcgctccacagcgacggcgactacctacgcatccaggacaactcgctccgtggcgccgcggcaaccgtggacgcggcgacgccggagaacatgcggacgctcgtcgggatcggggagcggatgctggcacagcgggtgtccagggtcaacgtggagacagggagcgaggtacgaaccggtgaccggagaaggaagcaatgccgatgccctcggtgggctcgctaggcagctctccgaggagaggagaacaaggctcgcgcgccgcgtctctgccatcaaccccagaagctctagatgtgcgccctacgatatctaagacaagtggctttactgtcaatcacatgcttgtaaataagtagactttattttaataaaatataaaaatatatat

SEQ ID NO: 4 - protein, 372 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINYFYMQNCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGSEVRTGDRRRKQCRCPRWAR

SEQ ID NO: 5 - DNA, 18 bp, Artificial Sequence
  GRMZM2G471240_nil.F1 primer
gtacgccgtgcgctaaca

SEQ ID NO: 6 - DNA, 20 bp, Artificial Sequence
  GRMZM2G471240_nil.R1 primer
tcgtacctccctgtctccac

SEQ ID NO: 7 - DNA, 19 bp, Artificial Sequence
  GRMZM2G471240_rwk.F1 primer
tacgccgtgcgctaacata

SEQ ID NO: 8 - DNA, 20 bp, Artificial Sequence
  GRMZM2G471240_rwk.R1 primer
gtacctcgctccctgtctcc

SEQ ID NO: 9 - DNA, 1358 bp, Artificial Sequence
  TALEN-induced MTL mutation in Event 39A individual ID 22808-3954 allele 1
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 10 - DNA, 1366 bp, Artificial Sequence
  TALEN-induced MTL mutation in Event 23A individual ID 22808-3924 allele 1
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggagaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 11 - DNA, 1358 bp, Artificial Sequence; identical to SEQ ID NO: 9
  TALEN-induced MTL mutation in Event 81A

SEQ ID NO: 12 - DNA, 1343 bp, Artificial Sequence
  TALEN-induced MTL mutation in Event 39A individual ID 22808-3954 allele 2
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 13 - DNA, 1363 bp, Artificial Sequence
  TALEN-induced MTL mutation in Event 23A ID 22808-3924 allele 2
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacaggaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 14 - DNA, 1360 bp, Artificial Sequence
  TALEN-induced MTL mutation in Event 38A individual ID 22808-4108 allele 1
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 15 - DNA, 1363 bp, Artificial Sequence; identical to SEQ ID NO: 13
  CRISPR-induced MTL mutation in Event 18A ID 22807-4016

SEQ ID NO: 16 - DNA, 1372 bp, Artificial Sequence
  CRISPR-induced MTL mutation in Event 27A individual ID 22807-4801 allele 1
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacaagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 17 - DNA, 1372 bp, Artificial Sequence; identical to SEQ ID NO: 16
  CRISPR-induced mutation in Event 27A individual ID 22807-4081 allele 1

SEQ ID NO: 18 - DNA, 1373 bp, Artificial Sequence
  CRISPR-induced MTL mutation in Event 76A individual ID 22873-3999
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacaaagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 19 - DNA, 1370 bp, Artificial Sequence
  CRISPR-induced mutation in Event 32A individual ID 22873-3991
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacgggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 20 - DNA, 18 bp, Artificial Sequence
  nucleotide sequence encoding guide RNA
gtcaacgtggagacaggg

SEQ ID NO: 21 - DNA, 1823 bp, Oryza sativa
  5′UTR (1)..(78)
  exon (79)..(444): exon 1
  Intron (445)..(536): intron 1
  exon (537)..(717): exon 2
  Intron (718)..(803): intron 2
  exon (804)..(967): exon 3
  Intron (968)..(1074): intron 3
  exon (1075)..(1662): exon 4
  3′UTR (1663)..(1823)
acagtgactagtgacaaacgatcgatcgatccctccatccacaaaccctcctcgatctcatcttccttcgtctcgtcaatggcggcgagctactcgtgccggcggacatgcgaggcgtgcagcacgagggcgatggccgggtgcgtggtgggcgagccggcgtcggcgccggggcagcgggtgacgttgctggcgatcgacggcggcggcatcaggggcctcatcccgggcaccatcctcgccttcctcgaggccaggctgcaggagctggatggccccgacgcgcgcctcgccgattacttcgactgcatcgccgggaccagcaccggcggcctcatcaccgccatgctcgccgcgcccggcgaccacggccgcccgctcttcgccgccagcgacatcaaccgcttctacctcgacaacggcccactcatcttcccacaaaagtaactgatcacctcgaattcgatctcctctcttcgatctctgcattatttgatttgattggggattgtgggcggcgtggcgtggcgtccaggaggtgcggcatggcggcggccatggcggcgctgacgaggccgaggtacaacggcaagtacctgcaggggaagatcaggaagatgctgggcgagacgagggtgcgcgacacgctgacgaacgtcgtcatccccacgttcgacgtcaggctgctccagccaaccatcttctccacatacgacgtgcgtgcgttgattccatccgcattggcgttggaatcagctgattgtttgattgatcgaacaattgatcggttaaaattttgcaggcgaagagcatgccgctcaagaacgcgctcctctccgacatctgcatcagcacatccgcggcgccgacctacctccccgcgcactgcttccagaccaccgacgacgccaccggcaaggtccgcgagttcgacctcatcgacggcggcgtcgccgccaacaacccggtaactaatcaatcaagcaatccatcaaacgaagatccacatgtgcattcctgtggtacaaatgctgatcgatcgatggatggatcgattttcgcgagaacgtacagacgatggtggccatgacgcagatcaccaagaagataatggtgaaggacaaggaggagctgtacccggtaaagccgtcggactgcggtaagttcctggtgctgtccgtgggcaccgggtcgacgtcggaccaggggatgtacacggcgaggcagtgctcgcggtgggggatcgtccggtggctgcgcaacaaggggatggcgcccatcatcgacatcttcatggcggccagctccgacctcgtcgacatccacgccgccgtcatgttccagtcgctgcacagcgacggcgactacctccgcatccaggacaacacgctccacggcgacgccgccacggtggacgccgccaccagggacaacatgcgggcgctcgtcgggatcggcgagcggatgctggcgcagcgggtgtcgagggtcaacgtcgagaccggcaggtacgtcgaggtgcccggcgccggcagcaacgccgacgcgctgaggggcttcgccaggcagctctccgaggagaggagggcgaggctaggtcggcgaaacgcctgcggcggcggcggcgaaggagagcccagcggcgtggcgtgcaagcgttagtaactgtacacgcatcatgctgacgcgatcttttttatttttctttttttttttttacctttctagcggacatggggaataacaagacgtgacagtagtgcaatcggtttgtaacgtgcgtataccaacattgatccatttcttcatcacagtttcagttc

SEQ ID NO: 22 - DNA, 1299 bp, Oryza sativa
atggcggcgagctactcgtgccggcggacatgcgaggcgtgcagcacgagggcgatggccgggtgcgtggtgggcgagccggcgtcggcgccggggcagcgggtgacgttgctggcgatcgacggcggcggcatcaggggcctcatcccgggcaccatcctcgccttcctcgaggccaggctgcaggagctggatggccccgacgcgcgcctcgccgattacttcgactgcatcgccgggaccagcaccggcggcctcatcaccgccatgctcgccgcgcccggcgaccacggccgcccgctcttcgccgccagcgacatcaaccgcttctacctcgacaacggcccactcatcttcccacaaaagaggtgcggcatggcggcggccatggcggcgctgacgaggccgaggtacaacggcaagtacctgcaggggaagatcaggaagatgctgggcgagacgagggtgcgcgacacgctgacgaacgtcgtcatccccacgttcgacgtcaggctgctccagccaaccatcttctccacatacgacgcgaagagcatgccgctcaagaacgcgctcctctccgacatctgcatcagcacatccgcggcgccgacctacctccccgcgcactgcttccagaccaccgacgacgccaccggcaaggtccgcgagttcgacctcatcgacggcggcgtcgccgccaacaacccgacgatggtggccatgacgcagatcaccaagaagataatggtgaaggacaaggaggagctgtacccggtaaagccgtcggactgcggtaagttcctggtgctgtccgtgggcaccgggtcgacgtcggaccaggggatgtacacggcgaggcagtgctcgcggtgggggatcgtccggtggctgcgcaacaaggggatggcgcccatcatcgacatcttcatggcggccagctccgacctcgtcgacatccacgccgccgtcatgttccagtcgctgcacagcgacggcgactacctccgcatccaggacaacacgctccacggcgacgccgccacggtggacgccgccaccagggacaacatgcgggcgctcgtcgggatcggcgagcggatgctggcgcagcgggtgtcgagggtcaacgtcgagaccggcaggtacgtcgaggtgcccggcgccggcagcaacgccgacgcgctgaggggcttcgccaggcagctctccgaggagaggagggcgaggctaggtcggcgaaacgcctgcggcggcggcggcgaaggagagcccagcggcgtggcgtgcaagcgttag

SEQ ID NO: 23 - protein, 432 aa, Oryza sativa
MAASYSCRRTCEACSTRAMAGCVVGEPASAPGQRVTLLAIDGGGIRGLIPGTILAFLEARLQELDGPDARLADYFDCIAGTSTGGLITAMLAAPGDHGRPLFAASDINRFYLDNGPLIFPQKRCGMAAAMAALTRPRYNGKYLQGKIRKMLGETRVRDTLTNVVIPTFDVRLLQPTIFSTYDAKSMPLKNALLSDICISTSAAPTYLPAHCFQTTDDATGKVREFDLIDGGVAANNPTMVAMTQITKKIMVKDKEELYPVKPSDCGKFLVLSVGTGSTSDQGMYTARQCSRWGIVRWLRNKGMAPIIDIFMAASSDLVDIHAAVMFQSLHSDGDYLRIQDNTLHGDAATVDAATRDNMRALVGIGERMLAQRVSRVNVETGRYVEVPGAGSNADALRGFARQLSEERRARLGRRNACGGGGEGEPSGVACKR

SEQ ID NO: 24 - DNA, 1371 bp, Zea mays
agttcatcactaatcacacttattgtgccctcgacgagtatctatagctagctcattaatcgattcgggggtgtgttgtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatgcagaactgcccgcgcatctttcctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 25 - DNA, 1375 bp, Zea mays; identical to SEQ ID NO: 3

SEQ ID NO: 26 - DNA, 1375 bp, Zea mays; identical to SEQ ID NO: 3

SEQ ID NO: 27 - protein, 401 aa, Zea mays; identical to SEQ ID NO: 2

SEQ ID NO: 28 - protein, 372 aa, Zea mays; identical to SEQ ID NO: 4

SEQ ID NO: 29 - protein, 372 aa, Zea mays; identical to SEQ ID NO: 4

SEQ ID NO: 30 - DNA, 15463 bp, Artificial Sequence
  Expression cassette of construct 22466
  promoter (214)..(2339)
  gene (2341)..(3018)
  terminator (3024)..(4036)
  enhancer (4044)..(4237): eFMV
  enhancer (4244)..(4536): e35S
  promoter (4544)..(8537): Ubi
  terminator (10009)..(11008): Ubi
  promoter (11028)..(12618): pPLA2
  exon (12619)..(12977): PLA2 exon1
  Intron (12978)..(13096): PLA2 intron1
  exon (13097)..(13193): PLA2 exon2
  Intron (13194)..(13388): PLA2 intron2
  exon (13389)..(13550): PLA2 exon3
  Intron (13551)..(13660): PLA2 intron3
  exon (13661)..(14248): PLA2 exon4
  terminator (14249)..(15244): tPLA2
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaaagtagcaaacaacaggttcatgtgcactataaaaagacaaaattctcgagtttcatcttttattccacataagccttatattttccattttcatatgatttttagtttaagtttgtgtcttaactttttcgttaatacgtaattctatgcattatggatgcgtgaagtatttttgtttaaaaaaatgaaatgtcaaaatacgttttgtgatctatttccatgttttcacctaacaggtggtttttactatatattctgccataactctagccttagatgtaaatcgaaaaaaaatgagagatgagctggagatagccttagatgaagcgtctgaaatataaaagaaagagtaatgttgaacgcagtaggtgtagcagctgtagttccatctctaggaaagggaactgcaatccgggctccgggcctcgcgcaatctggcctgtcgtgtagatgcagccctgtccatgacggcccaagcaacgcccgcggctctcgatccaccacggaacccactccgacacacactgacacacacatgctggatgtggatgtgctgtccaattattagtagcaattcggtaggcacaggcacgtactggccggtgttttagctgtaagtaccgaaccaatcacggttaagaaccgattaatccgtgcccagccgccgagtgcgttcgtacgtgcatcggatgcactgcatgaattgagagcatcatcatatcatacgcaggagtagtacgacgccgctgctgtcttgtccggctaatgctttgctcacagattagtccatcgcccacggtcggtgtggtgtggatcgctgatgccactgctttttgtttggtttttattcccctgataatcctccgcgtccctgaatgtatctatttattttcattccgaaatccctttcacgaaaaagaaaacgaataaaaagagagttacgaatacgcttccggcggcccacatcaccttccagcgaacatcgcgccgcgctgacgtgtcgcccatcgcggccgtccatatcgccatccgacgaccgtggaagctggcagcggccgctccgttccgtcgaaggggcaggtcagtcaggtcacccacacggccacacccgcgcgggggatacgcggtggaaaacccggcgaccacatcaaaacacgaggcgtctcccgcaggactggtcactcggcacgcaggcagaggcagcacagcagcagccagctccatccatcctctttcccctcctcgcttcgcttcctcggcggattcctcctccctcggccgtccccgtccccttcttcgccgcgccagctcgcccgagttggtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagaccagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttcgatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcaggcaaacccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggagctcgcatcatgatcatgcatcatggactcggcctactactgtggatttgtatgccattatagacttggtgctgtgaaagactgcttgatgatttgcgggtttgttgctgtgtaaaaaaaggtcccttggctcccagaagaccatgaaggttcggatctatcatgtaattccttgttatctgccaattatgtatggactatggacatgtgttgcgctgttcaacttactactacaaataagtaatcgatatgttcccttcccatgtctcggtgacaattgtctggagaagcttaggggtcgtttgtttgggattatgtctggagaaacttattttaaactaagtgtgagttcaagttaagttagattatataatctaggcagattataattccaagcgaacaggtccttagtgtttttggaaaatcctaggtgttcttttggctacattgttgtgtgtgcagatcccttgttggtctgtaagcgtggggaagtaagaatcgtccgtttctactgaagacctgctcgagttaggcaccgaggatgccggtaaccaaacagagcaatagtgtctctgtgggcacagtggagtgtgaatctgtgtgatgcaaatccgtcatttgtttagcaaaatttccagcgttgcatgatgcagtttctttaacacggacttaagggaagggaaaaaaatgttgagccaggagatccttcaatgtgttagactgacgtgatagccaactaaaccacgacgcaatgttgtcgttaatgacaaaaaaactatttgttcctaaatccttggcgacattgcatggctgtctcatgagataatggtctcatctcttatttatctcttatttatagccggaagtggtagtgacccctgcttgattgctcgtatgccatctcaagttctcaaccgtgtcgagcagccattttcccatctcaagcgcatcatcgtttcgtttgacctcatctgctatcctgctcctagtgcaaatcacatgcgacagaaagtgtgcggacccagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcaagctttggaaaaccaaaacaacgaataagcaaactgcaggaaaagtatgcagtggaaaccaacccagattcggacgataggaaagtatcaagtgaatgatttgccaggaaaaggagaggggtaaaaaggggcgaagatttagaagatctaaagcacaagaaccagagattagattgaacaatagggaacttggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaagctataaagaatacattaacattcagtgaaacagcatgctgtcttcttctttttttatgcacaacagagcatacatatataccttcccaggctgaggacttggcggaggagagccgcggataggttggcggtgcagacggtctggacgggcccgaagacggagacgaacagcgggcccttcctgcccaggcaccacgcttggaacgccaagcacgcgccaaccgcggccccgccgaggacgacgatccccgcgacaagcgtggcgtcgatcctgggcgaccccaagccgaggaacctcccttccaggacgagccgtaggacggcggtgagagaggcacccgtcgcggaggtggcgcagcacaaggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggtaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctctgaacgcacacaccgcccggtctccgatctccgatccccgatcccctcgtcgatcctaggtacggcgaccatcctaccccccccccccccccctctctctctgccttctctagatcggcgatccgatccatgcttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatccagatcttcgtaaaccatggctctggtggctaggccagtgctgtctgctagagtggctgcttctaggccaagggtggccgctagaaaggctgtgagggtgagcgctgccagtcacggcgctagctctaggccagccaccgctaggaagtccagcggcctgagcggcaccgtgcgcatcccaggcgacaagagcatcagtcaccgcagcttcatgttcggcggcctggccagcggcgagactaggatcaccggcctgctcgagggcgaggacgtgatcaacaccggcaaggccatgcaggctatgggcgctcgcatccgcaaggagggcgacacctggatcatcgacggcgtgggtaatggcggcctgctggccccagaggccccactggacttcggcaacgccgccaccggctgcaggctgactatgggcctggtgggcgtgtacgacttcgacagcacctttatcggcgacgccagcctgaccaagaggccgatgggcagggtgctgaacccgctgcgcgagatgggcgtgcaggtcaagagcgaggacggcgacaggctgccggtgaccctgaggggcccaaagaccccgaccccgatcacctaccgcgtgccaatggccagcgcccaggtgaagtcagccgtgctgctggccggcctgaacaccccaggcatcaccaccgtgatcgagccgatcatgacccgcgatcacaccgagaagatgctgcagggcttcggcgccaacctgaccgtcgagactgacgctgacggcgtgaggaccatcaggctcgagggcaggggcaagctgaccggccaggtgatcgacgtgccgggcgacccaagcagcaccgccttcccactggtggccgccctgctggtgccaggctctgacgtgaccatcctgaacgtgctgatgaacccgagccgcaccggcctgatcctgaccctgcaggagatgggcgccgatatcgaggtgatcaacccgaggctggctggcggcgaggacgtcgccgacctgagggtgaggtccagcaccctgaagggcgtgaccgtgccagaggacagggccccgagtatgattgacgagtacccgatcctggccgtggccgctgccttcgccgagggcgccaccgtgatgaacggcctcgaggagctgcgcgtgaaggagagcgacaggctgagcgctgtggccaacggcctgaagctgaacggcgtggactgcgacgagggcgagactagcctggtggtgaggggcaggccagacggcaagggcctgggcaacgcttccggcgctgccgtggccacccacctggatcacaggatcgccatgtcgttcctcgtgatgggcctcgtgagcgagaacccggtgaccgtggacgacgccaccatgatcgccaccagcttccccgagttcatggacctgatggccggcctgggcgctaagatcgagctgagcgacaccaaggccgcctgatcatctagagctcgccaaggttcaattaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctcccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattactaattagctaacggaccctatttgtactcattccatgtctcataaactttgggcaccatccatccaacacatccaatctaaacacaccaaacgatggggaatggaaagagcagtattcgattcaacaatggcaaacaaatatcactgaattagaccaagaataaacctaattagacaacgacctcccaaccatcattcgtcaggctgtaaagaagataaagctgccatggggcatggatcaagcagaacaccagagatgaatccaaacacacagaaaatcacgcgcgctgtctacaatgacaacaagccccacatttcattgcagtacactgggctacaaaggcacgtacaacaaagagctagggaaacattgcggagggcacgagagagcagctaacttgacaatatagcagactgagcttgcactgttagcaggcgaggaagggaatcatggggacggagaatggggtccatgcccgcgaaggagaaggcggacgccgccacggtggcaccggcgcacgcgcacacagggaacccgcacaggcagccatggatgctgcctcgccattgcgccggtcgtctctgccacgctcctctctctctcccgctgcatcgccgtggatggggcaagcagagagcagggactgcgacgatctgggcggaggactcgccttggagagcgcggacgcagacgggattctagggagagagcgaagacggggcgcgcgcggcgctcgcgcggcgtggtggcggcgagattagcgggggtggggggagggcggagccgtggtgagggtgtggacgccctccttaccctcttaagtagtagtagagatataatccgttccaaaatatccatccgttcaatttatatttcgtttgatctttttaccctaaatttgattgactcatcttattaaaaaagttcataactattattaatctttattgagatatcatttagcatataatatactttaagtgtggttttagattttttttaaaaaaaaaaattcgcaaaaattaaatgaaacgacccaatcaaacttgaaaagtaaaactaattataaatttgaacggaaggagtaagaggatgtttgaatgtactagagctaatagttggttgctttaaaatttgctagtagaattagctagctaataaatatctagataactattagctaatttgctaaaacagctaatagttgaactattagctagattgtttggatgtattcggctaattttaatggctaactattagctatagtacaatattcaaacacctcctaattaaaatggacaaatatctcttcttttggtcccttgcgttagatttttcatatctccttatttagtataaaagaatcatcaaaaagtggacaacccctagtggaacaccattttagtagtggttgcatgaaacctttcgcgcaccagtttctatgtgtcactctaaaaatgggacagcatgtacgtagtgcctatatatatacaagtcatctatcgttgcctcctcagttcatcactaatcacacttattgtgccctcgacgagtatctatagctagctcattaatcgattcgggggtgtgttgtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacgggccggaggcgaggctggcggactacttcgactacatcgccggaaccagcaccggcggtctcatcaccgccatgctcaccgcgcccggcaaggacaagcggcctctctacgctgccaaggacatcaaccacttttacatgcagaactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcggccgccatgtccgcgctgaggaagccaaagtacaacggcaagtgcatgcgcagcctgattaggagcatcctcggcgagacgagggtaagcgagacgctgaccaacgtcatcatccctgccttcgacatcaggctgctgcagcctatcatcttctctacctacgacgtacgtacgtcgtcacgaatgattcatctgtacgtcgtcgcatgcgaatggctgcctacgtacgccgtgcgctaacatactcagctctttcctatctgctgcgccaatttgcaggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccggtaactgactagctaactggaaaacggacgcacagactccatgtccatggcggcccacaaggtcgatgctaattgttgcttatgtatgtcgcccgattgcacatgcgtagacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggactaccgtgaatgactatagaaccaatcatactagtataaaatatatgatgacactacgggagagacaaactttgtctggcgctaaatattttgccgagtgtgaattcacgggcactaggcaaagatcttctttgccgagtgttacgctgggcaaagtaagacactaggtaaatcagtcatttgccgagtgtccgccactaggcaaagcaaaacactggcaaatcaaaagtttacctagtgccagacactaggcaaaaaaaaaacgctcggcaaatcggaagtttccctagtgccagacactagacaaagaaaaacacttgataaactagcgtcgtcagctaacaccatccaccaaccgttaacgttgccgagtatctgacttcgacactcggcaaagaaggtctctttgcctagtgtcggtctggaacactaggcaaagaggcactttacctagtgtcgtattttgacactcagtaaaataattttttttctttctgcttccaaactttttatgatgtgttcctatagcacctagaactacatgtcaagttttggtaaaatttttgaagtttttgctatatttacttaatttattttatttaattgaatttcttttgataattcaaatttgaactcggcaaggtaagaagcgagggtagcctggaaacacactttgcctagtgttacactcggtacaggagcctcccctgcctagtgctgcactcgacaaaagattcgcctttgcctagcgctgcactcggcacaggagtcgcctttgcctagtgctgcactaggcaaagcctccgttaccgtgccttccatcgtcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 31 - DNA, 15757 bp, Artificial Sequence
  Expression cassette of construct 22467
  promoter (214)..(2339)
  gene (2341)..(3018)
  terminator (3024)..(4036)
  enhancer (4044)..(4237): eFMV
  enhancer (4244)..(4536): e35S
  promoter (4544)..(8537): Ubi
  gene (8538)..(9995): EPSPS
  terminator (10009)..(11008): Ubi
  gene (11028)..(15538): GRMZM2G062320
  promoter (11028)..(12596): pPGM
  exon (12597)..(12834): PGM exon1
  Intron (12835)..(12974): PGM intron1
  exon (12975)..(13198): PGM exon2
  Intron (13199)..(13295): PGM intron2
  exon (13296)..(13382): PGM exon3
  Intron (13383)..(13718): PGM intron3
  exon (13719)..(13787): PGM exon4
  Intron (13788)..(14102): PGM intron4
  exon (14103)..(14204): PGM exon5
  Intron (14205)..(14304): PGM intron5
  exon (14305)..(14424): PGM exon6
  Intron (14425)..(14492): PGM intron6
  exon (14493)..(14540): PGM exon7
  terminator (14541)..(15538): tPGM
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaaagtagcaaacaacaggttcatgtgcactataaaaagacaaaattctcgagtttcatcttttattccacataagccttatattttccattttcatatgatttttagtttaagtttgtgtcttaactttttcgttaatacgtaattctatgcattatggatgcgtgaagtatttttgtttaaaaaaatgaaatgtcaaaatacgttttgtgatctatttccatgttttcacctaacaggtggtttttactatatattctgccataactctagccttagatgtaaatcgaaaaaaaatgagagatgagctggagatagccttagatgaagcgtctgaaatataaaagaaagagtaatgttgaacgcagtaggtgtagcagctgtagttccatctctaggaaagggaactgcaatccgggctccgggcctcgcgcaatctggcctgtcgtgtagatgcagccctgtccatgacggcccaagcaacgcccgcggctctcgatccaccacggaacccactccgacacacactgacacacacatgctggatgtggatgtgctgtccaattattagtagcaattcggtaggcacaggcacgtactggccggtgttttagctgtaagtaccgaaccaatcacggttaagaaccgattaatccgtgcccagccgccgagtgcgttcgtacgtgcatcggatgcactgcatgaattgagagcatcatcatatcatacgcaggagtagtacgacgccgctgctgtcttgtccggctaatgctttgctcacagattagtccatcgcccacggtcggtgtggtgtggatcgctgatgccactgctttttgtttggtttttattcccctgataatcctccgcgtccctgaatgtatctatttattttcattccgaaatccctttcacgaaaaagaaaacgaataaaaagagagttacgaatacgcttccggcggcccacatcaccttccagcgaacatcgcgccgcgctgacgtgtcgcccatcgcggccgtccatatcgccatccgacgaccgtggaagctggcagcggccgctccgttccgtcgaaggggcaggtcagtcaggtcacccacacggccacacccgcgcgggggatacgcggtggaaaacccggcgaccacatcaaaacacgaggcgtctcccgcaggactggtcactcggcacgcaggcagaggcagcacagcagcagccagctccatccatcctctttcccctcctcgcttcgcttcctcggcggattcctcctccctcggccgtccccgtccccttcttcgccgcgccagctcgcccgagttggtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagaccagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttcgatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcaggcaaacccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggagctcgcatcatgatcatgcatcatggactcggcctactactgtggatttgtatgccattatagacttggtgctgtgaaagactgcttgatgatttgcgggtttgttgctgtgtaaaaaaaggtcccttggctcccagaagaccatgaaggttcggatctatcatgtaattccttgttatctgccaattatgtatggactatggacatgtgttgcgctgttcaacttactactacaaataagtaatcgatatgttcccttcccatgtctcggtgacaattgtctggagaagcttaggggtcgtttgtttgggattatgtctggagaaacttattttaaactaagtgtgagttcaagttaagttagattatataatctaggcagattataattccaagcgaacaggtccttagtgtttttggaaaatcctaggtgttcttttggctacattgttgtgtgtgcagatcccttgttggtctgtaagcgtggggaagtaagaatcgtccgtttctactgaagacctgctcgagttaggcaccgaggatgccggtaaccaaacagagcaatagtgtctctgtgggcacagtggagtgtgaatctgtgtgatgcaaatccgtcatttgtttagcaaaatttccagcgttgcatgatgcagtttctttaacacggacttaagggaagggaaaaaaatgttgagccaggagatccttcaatgtgttagactgacgtgatagccaactaaaccacgacgcaatgttgtcgttaatgacaaaaaaactatttgttcctaaatccttggcgacattgcatggctgtctcatgagataatggtctcatctcttatttatctcttatttatagccggaagtggtagtgacccctgcttgattgctcgtatgccatctcaagttctcaaccgtgtcgagcagccattttcccatctcaagcgcatcatcgtttcgtttgacctcatctgctatcctgctcctagtgcaaatcacatgcgacagaaagtgtgcggacccagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcaagctttggaaaaccaaaacaacgaataagcaaactgcaggaaaagtatgcagtggaaaccaacccagattcggacgataggaaagtatcaagtgaatgatttgccaggaaaaggagaggggtaaaaaggggcgaagatttagaagatctaaagcacaagaaccagagattagattgaacaatagggaacttggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaagctataaagaatacattaacattcagtgaaacagcatgctgtcttcttctttttttatgcacaacagagcatacatatataccttcccaggctgaggacttggcggaggagagccgcggataggttggcggtgcagacggtctggacgggcccgaagacggagacgaacagcgggcccttcctgcccaggcaccacgcttggaacgccaagcacgcgccaaccgcggccccgccgaggacgacgatccccgcgacaagcgtggcgtcgatcctgggcgaccccaagccgaggaacctcccttccaggacgagccgtaggacggcggtgagagaggcacccgtcgcggaggtggcgcagcacaaggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggtaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctctgaacgcacacaccgcccggtctccgatctccgatccccgatcccctcgtcgatcctaggtacggcgaccatcctaccccccccccccccccctctctctctgccttctctagatcggcgatccgatccatgcttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatccagatcttcgtaaaccatggctctggtggctaggccagtgctgtctgctagagtggctgcttctaggccaagggtggccgctagaaaggctgtgagggtgagcgctgccagtcacggcgctagctctaggccagccaccgctaggaagtccagcggcctgagcggcaccgtgcgcatcccaggcgacaagagcatcagtcaccgcagcttcatgttcggcggcctggccagcggcgagactaggatcaccggcctgctcgagggcgaggacgtgatcaacaccggcaaggccatgcaggctatgggcgctcgcatccgcaaggagggcgacacctggatcatcgacggcgtgggtaatggcggcctgctggccccagaggccccactggacttcggcaacgccgccaccggctgcaggctgactatgggcctggtgggcgtgtacgacttcgacagcacctttatcggcgacgccagcctgaccaagaggccgatgggcagggtgctgaacccgctgcgcgagatgggcgtgcaggtcaagagcgaggacggcgacaggctgccggtgaccctgaggggcccaaagaccccgaccccgatcacctaccgcgtgccaatggccagcgcccaggtgaagtcagccgtgctgctggccggcctgaacaccccaggcatcaccaccgtgatcgagccgatcatgacccgcgatcacaccgagaagatgctgcagggcttcggcgccaacctgaccgtcgagactgacgctgacggcgtgaggaccatcaggctcgagggcaggggcaagctgaccggccaggtgatcgacgtgccgggcgacccaagcagcaccgccttcccactggtggccgccctgctggtgccaggctctgacgtgaccatcctgaacgtgctgatgaacccgagccgcaccggcctgatcctgaccctgcaggagatgggcgccgatatcgaggtgatcaacccgaggctggctggcggcgaggacgtcgccgacctgagggtgaggtccagcaccctgaagggcgtgaccgtgccagaggacagggccccgagtatgattgacgagtacccgatcctggccgtggccgctgccttcgccgagggcgccaccgtgatgaacggcctcgaggagctgcgcgtgaaggagagcgacaggctgagcgctgtggccaacggcctgaagctgaacggcgtggactgcgacgagggcgagactagcctggtggtgaggggcaggccagacggcaagggcctgggcaacgcttccggcgctgccgtggccacccacctggatcacaggatcgccatgtcgttcctcgtgatgggcctcgtgagcgagaacccggtgaccgtggacgacgccaccatgatcgccaccagcttccccgagttcatggacctgatggccggcctgggcgctaagatcgagctgagcgacaccaaggccgcctgatcatctagagctcgccaaggttcaattaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctcccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattactaattagctaacggaccccttgcctcggttacaattgagtttgtcataagaaagaatgagaaagaaaagaagcaatccaagcgcaagagctcaaaggaacacaagtcactctctcactagtcactattgattggaattgaactagggacttgggagaggattttatctctttggtgtgtcttgtattgaatgctatagctcttgtaatgtgtagaatgttggaaacttggatgccattgaatgtggggtggttggggtatttatagccccaaccaccacaatgtggtcgttggaagtctgctgtcgcatggcgcaccggatagtccgatgcgccaccggacactgtccggtgcgccagccacgtcagccagccgttggggttcgaccgttggagctctgacttgtggggcctctgggctgtccggtggtgcaccggacaggtcctgtagactgtccggtgcaccaactgcgcgtgctctgacctctgcgcgcgcaggcgcgcattaaatgcgttgcagtcgaccgttgcgcttgaagtagtcgttgctccgctggcacaccggacagtgtccggtgaattatagcggagcggcctccattttcccgaaggtagcgagttcagcgtcgagttccctggtgcaccggacactgtccggtggcacaccggacagtccggtgcgccagaccagggcacacttcggttgtctttggctctctttgtttgaaccctttcttggtctttttattggtctattgtgaacctttggcacctgtaaaacttataatctagagtaaactagttagtccaattatttgtgttgggcaattcaaccaccaaaatcaattaggaaaaggtgtaagcctatttccctttcaggaggcgtacgtgagagggagaggtgaaaaggaacaacgcgtataccagataaggtcccacagcctaagtaggtagccttctaatatctctactaactattaaggagagagtgtagactgcccccgctccctacccaacgccccccgctacctgttcaccgcgcgccagcgaaacctccgcacgcccactgcccatctgttccccgtgcgccagcgaaacatccgcacgcccgcggcccgcctgttccccgcgcatcccgctgcacgacttctgctaccgcaacggccacccacgcacgcccgcctgttcaccgcgcatcccgctgacctccccttcacgctcgcacacgctccgttcccccaccccaccgcaatccccgacgctataagagcggtaaccaactccatctccctggtgccacgcattgttgagttcttaaggtgcgtttcgttgaggacttgttcatttttgttggtcatgtattccattttactgctctaccattttgtggaataaagggaggaatgttttcactagaagagttcatcaatcttatgttggtttcttggatcagttttgctctatggctaaatggtcgaattgagcctatttcattataaagttagcgagcgaataattgttcagcctcttcctagaactcattaccagtagaatcagttactaactgcttttctttttcttggattagaatggctggggctatctctcaccatgcgctagcattttcacaatcccactggtgcagtgcgaagaactctagattcggaaagaggacgggcaatgctcgcctggtttatctaaaaggaagatgtggttcaggcagcagaaaactgggtttgatgtgggcctcgagctcgcagtcttctgtcatggagccgacgcacctaccatctgatggcaacagcagccacaccccaaaaaaatcaagtaattttaacgacctcctatggtggttatttgtttttaatttgagaaaactatccatttgacacatttaactttgggcttctcagaatttgggggcatataataagatctgctaatctgttatctctatgtcgttgtaggtgaaagcgctcttatattgatttggcatggtgaatccctgtggaacgagaaaaatctatttcctggctgcatcgatgtacccctgacaccgaagggtgttgaggaggccattgaggcaggtaaaaggatatgcaatatcccaatcgatgtgatatatacttcatcactgatttgtgctcagatgaccgcaatgcttgccatgatgcagcatcgacgcaagaaggtttgtgtctttcctttgaaattccagtaatttcttctagcatttgtatgaacttgccggagaaatcatgctttgctggtgatatatgtatttatagatcctagttatcacgcataatgagagtgaacaagctcacaggtggagtcagatatacagtgaggagacaatgaaacagtccattcctgtcatcacagcttggcaattgaatgaacggatgtaatactttctccatactctttgatttgctaattactccctctgtctcaaaatagtattaattttagctcttgatttttatgtctatattcaaatagatgatgataaatctagattctagacacaaatataaaacatatacatcaagtattatatgaatctattaatttactaagaccaattttaatttgggacagagggagtatacgattataatagttgtttgactgtgcttctctttaaatatcccttgacatttctaggtatggtgagctacaaggccttaacaagcaagaaactgtagatcgatttggcaaagaacaagttcatgagtggcgccgcagttatgatattcctccgccaaatggagaaagtctagagaagtgtgctgagagagctgttgcttatttcaaagatcaggcacatctagcaaggccactttacactaattgaaagatacactttttacttgggttattggtcttgctgcagtattggtatgcatgctaaaggttattcttgaatcgatgaattcctctactatgggatgcagaaatgcatgtgcttagttttctttctattgtgctagctcatatcaaatttataacctgaattttttatttatgttcgactctaaaaaacagttttttctagctcgatttgacctatagtaatttttccgtaatagattattccacaacttgtggctggaaaacatgtgatggttgctgcacatgggaattcacttcgttcaattataatgcatctggacaaattaacttctcagaaggtaattcactgtcgtttttgtctttccatcaaaaaggactcggctaaacagaacatgtagcattatgttaagtttgggagtgagcctttcgtcccttcaggtaataagccttgagctgtctactggcattcccatgctttacatattcaaagagggaaagtttattcgacgtgggactccagtaggaccttcggaggccagtgtttatgcttataccagggtaagattctttcccccacatgttctaccataggacgatactccagtttacaaaccttatctgtacagaccaaacgatttgctgagcacattacatttcagaacaaattggcctagaagataaggggtgtttggtttgagaaatcactctattcaaaatgagatggtgtatcatgggtccatttctcaaatttggtgggatgaccctattcctcatattagtactaactaggtgagtgtccgtgcgttgcaacgggaacatataataacatgataacttatatacaaaatgtgtcttatattgttataagaaaatgtttcataatccatttgtaatcctggccatacataaattttgttattttaatttagctgtttcactactacattgcaaccatcagtatcatgcagacttcgatatatgtcacgatttgcatagtcttatcattgaagagcacgtgtcacacctaccggtagaagttccctcgtacattgtcagggggacctcatggtcgtcgctaaagccggcgactctctggttgttctgggcaccgcatccgacgacaacaccgtcacgctgtccagctcatcatccacctgaagatcaacctgccacgtaagtcggtagtgaccggccatgaattcttgtgcgctggggcaacgggcgttgcattccgttgtgtgagtgttttagaacaagatgtatgtaaaggagtagcacatccggtggtgcaatggctaggtgtactacctcgctgatgagctcgcggtgcacttcgtttagcagcccagccaagagtcatcggtacttgccatgtcgtgcgtgttcgacgactactatatcaaggactgtggcgtcctcttggcgccagaggtgaggagtaccgacagcaatgacctctcgccatcgtcggggtagcttttgtgctggcccgcctttaattctcttcgtaaacacatacacttgccttttttgtttaagcaagtgagtatagtggtgcgtacctgatgactgacaatgtacgaggaaacttctactggcaggtgtggcacgtgctcttcaatgatgagaccatgtaaatcgtgacatatatcgaagtctgcatgatactgatcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 32 - DNA, 15355 bp, Artificial Sequence
  Expression cassett of construct 22503
  enhancer (214)..(407): eFMV
  enhancer (414)..(706): e35S
  promoter (714)..(4707): Ubi
  gene (4708)..(6165): EPSPS
  terminator (6179)..(7178): Ubi
  promoter (7198)..(9285): pPLA2m
  stem_loop (9298)..(10212): targeting exon2 of PLA2
  terminator (10223)..(11218): tPLA2
  promoter (11226)..(13349)
  gene (13352)..(14131)
  misc_feature (14039)..(14128)
  terminator (14138)..(15136)
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcaagctttggaaaaccaaaacaacgaataagcaaactgcaggaaaagtatgcagtggaaaccaacccagattcggacgataggaaagtatcaagtgaatgatttgccaggaaaaggagaggggtaaaaaggggcgaagatttagaagatctaaagcacaagaaccagagattagattgaacaatagggaacttggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaagctataaagaatacattaacattcagtgaaacagcatgctgtcttcttctttttttatgcacaacagagcatacatatataccttcccaggctgaggacttggcggaggagagccgcggataggttggcggtgcagacggtctggacgggcccgaagacggagacgaacagcgggcccttcctgcccaggcaccacgcttggaacgccaagcacgcgccaaccgcggccccgccgaggacgacgatccccgcgacaagcgtggcgtcgatcctgggcgaccccaagccgaggaacctcccttccaggacgagccgtaggacggcggtgagagaggcacccgtcgcggaggtggcgcagcacaaggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggtaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctctgaacgcacacaccgcccggtctccgatctccgatccccgatcccctcgtcgatcctaggtacggcgaccatcctaccccccccccccccccctctctctctgccttctctagatcggcgatccgatccatgcttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatccagatcttcgtaaaccatggctctggtggctaggccagtgctgtctgctagagtggctgcttctaggccaagggtggccgctagaaaggctgtgagggtgagcgctgccagtcacggcgctagctctaggccagccaccgctaggaagtccagcggcctgagcggcaccgtgcgcatcccaggcgacaagagcatcagtcaccgcagcttcatgttcggcggcctggccagcggcgagactaggatcaccggcctgctcgagggcgaggacgtgatcaacaccggcaaggccatgcaggctatgggcgctcgcatccgcaaggagggcgacacctggatcatcgacggcgtgggtaatggcggcctgctggccccagaggccccactggacttcggcaacgccgccaccggctgcaggctgactatgggcctggtgggcgtgtacgacttcgacagcacctttatcggcgacgccagcctgaccaagaggccgatgggcagggtgctgaacccgctgcgcgagatgggcgtgcaggtcaagagcgaggacggcgacaggctgccggtgaccctgaggggcccaaagaccccgaccccgatcacctaccgcgtgccaatggccagcgcccaggtgaagtcagccgtgctgctggccggcctgaacaccccaggcatcaccaccgtgatcgagccgatcatgacccgcgatcacaccgagaagatgctgcagggcttcggcgccaacctgaccgtcgagactgacgctgacggcgtgaggaccatcaggctcgagggcaggggcaagctgaccggccaggtgatcgacgtgccgggcgacccaagcagcaccgccttcccactggtggccgccctgctggtgccaggctctgacgtgaccatcctgaacgtgctgatgaacccgagccgcaccggcctgatcctgaccctgcaggagatgggcgccgatatcgaggtgatcaacccgaggctggctggcggcgaggacgtcgccgacctgagggtgaggtccagcaccctgaagggcgtgaccgtgccagaggacagggccccgagtatgattgacgagtacccgatcctggccgtggccgctgccttcgccgagggcgccaccgtgatgaacggcctcgaggagctgcgcgtgaaggagagcgacaggctgagcgctgtggccaacggcctgaagctgaacggcgtggactgcgacgagggcgagactagcctggtggtgaggggcaggccagacggcaagggcctgggcaacgcttccggcgctgccgtggccacccacctggatcacaggatcgccatgtcgttcctcgtgatgggcctcgtgagcgagaacccggtgaccgtggacgacgccaccatgatcgccaccagcttccccgagttcatggacctgatggccggcctgggcgctaagatcgagctgagcgacaccaaggccgcctgatcatctagagctcgccaaggttcaattaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctcccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattactaattagctaacggaccctatttgtactcattccatgtctcataaactttgggcaccatccatccaacacatccaatctaaacacaccaaacgatggggaatggaaagagcagtattcgattcaacaatggcaaacaaatatcactgaattagaccaagaataaacctaattagacaacgacctcccaaccatcattcgtcaggctgtaaagaagataaagctgccttggggcatggatcaagcagaacaccagagatgaatccaaacacacagaaaatcacgcgcgctgtctacaatgacaacaagccccacatttcattgcagtacactgggctacaaaggcacgtacaacaaagagctagggaaacattgcggagggcacgagagagcagctaacttgacaatatagcagactgagcttgcactgttagcaggcgaggaagggaatcatggggacggagaatggggtccatgcccgcgaaggagaaggcggacgccgccacggtggcaccggcgcacgcgcacacagggaacccgcacaggcagccaaggatgctgcctcgccattgcgccggtcgtctctgccacgctcctctctctctcccgctgcatcgccgtggatggggcaagcagagagcagggactgcgacgatctgggcggaggactcgccttggagagcgcggacgcagacgggattctagggagagagcgaagacggggcgcgcgcggcgctcgcgcggcgtggtggcggcgagattagcgggggtggggggagggcggagccgtggtgagggtgtggacgccctccttaccctcttaagtagtagtagagatataatccgttccaaaatatccatccgttcaatttatatttcgtttgatctttttaccctaaatttgattgactcatcttattaaaaaagttcataactattattaatctttattgagatatcatttagcatataatatactttaagtgtggttttagattttttttaaaaaaaaaaattcgcaaaaattaaatgaaacgacccaatcaaacttgaaaagtaaaactaattataaatttgaacggaaggagtaagaggatgtttgaatgtactagagctaatagttggttgctttaaaatttgctagtagaattagctagctaataaatatctagataactattagctaatttgctaaaacagctaatagttgaactattagctagattgtttggatgtattcggctaattttaatggctaactattagctatagtacaatattcaaacacctcctaattaaaatggacaaatatctcttcttttggtcccttgcgttagatttttcatatctccttatttagtataaaagaatcatcaaaaagtggacaacccctagtggaacaccattttagtagtggttgcatgaaacctttcgcgcaccagtttctatgtgtcactctaaaaatgggacagcatgtacgtagtgcctatatatatacaagtcatctatcgttgcctcctcagttcatcactaatcacacttattgtgccctcgacgagtatctatagctagctcattaatcgattcgggggtgtgttgtcgaaggcggcattggcgagctactcgtcgcggcgtccaagcaatacctgtagcacgaaggcgatcgccgggagcgtggtcggcgagcccgtcgtgctggggcagagggtgacggtgctgacggtggacggcggcggcgtccggggtctcatcccgggaaccatcctcgccttcctggaggccaggctgcaggagctggacgcaccggaggcgaggctggcggactacttcgactacatcgccggaaccagcaccggcggtctcatcaccgccttgctgaccgcgcccggcaaggacaagcggcctctctaggctgccaaggacatcaaccacttttacatccataactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcgaaaaccccatggagatctgaacaggcttgcggccgccgtgtccgcgctgaggaagccaaagtacaacggcaagtgcatgcgcagcctgattaggagcatcctcggcgagacgaggggtcgactcgggagcatgccctttgtgcttatgcctccgttctgccttctgacgaatttggtactggaagcagatgagttttggttcactatcattctgaatttacacctgcgcttgctgtcagactaggcaaccaagtgacttttgtgactttgatcatgttcagtgtgtttccaagtcctaatcaatcaaaaagaaaaacagtttgttaacgattgtttgccatgtctatataataaagttgcttttatagtagcttagaattcaatcggccaactttatctcgtacgctgacagtaaaggtacatttaaaaggtgacaatggatagtctaatacttgaactgacaatagagacacattacatgtcagttgattaagtttgtaacagaaaaataaacaatactacataattgcaaagtttctttgatgtctttctttcaagaaacacaaatatatcaatgctacagtattgctgatgaatttatccatgttgagatgtttttctggtttctgatctgatcagtctcaattggtgtgctgtttcattttcatttgctgatgatcgtccgagtagttaattcttactaatatttagataatttggcatacaagcgaatcacgtagaacatgatacttttgaatgaatttatcaaagttttatcacttggtgagttgtttcatggttttcctactgatgtctcttcttcagatttctcgagccctcgtctcgccgaggatgctcctaatcaggctgcgcatgcacttgccgttgtactttggcttcctcagcgcggacatggcggccgcaagcctgctcggatccatgggacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggactaccgtgaatgactatagaaccaatcatactagtataaaatatatgatgacactacgggagagacaaactttgtctggcgctaaatattttgccgagtgtgaattcacgggcactaggcaaagatcttctttgccgagtgttacgctgggcaaagtaagacactaggtaaatcagtcatttgccgagtgtccgccactaggcaaagcaaaacactggcaaatcaaaagtttacctagtgccagacactaggcaaaaaaaaaacgctcggcaaatcggaagtttccctagtgccagacactagacaaagaaaaacacttgataaactagcgtcgtcagctaacaccatccaccaaccgttaacgttgccgagtatctgacttcgacactcggcaaagaaggtctctttgcctagtgtcggtctggaacactaggcaaagaggcactttacctagtgtcgtattttgacactcagtaaaataattttttttctttctgcttccaaactttttatgatgtgttcctatagcacctagaactacatgtcaagttttggtaaaatttttgaagtttttgctatatttacttaatttattttatttaattgaatttcttttgataattcaaatttgaactcggcaaggtaagaagcgagggtagcctggaaacacactttgcctagtgttacactcggtacaggagcctcccctgcctagtgctgcactcgacaaaagattcgcctttgcctagcgctgcactcggcacaggagtcgcctttgcctagtgctgcactaggcaaagcctccgttaccgtgccttccatcgtcggaccctggagaggcatgaacccatcatgtatactcctggacttggaagaggaaatgtcaaccaaagtgaaaggttttcttctagttgctgctaagagataaattgaatactagatctctcctaagacatcggggcattcgtctggactactacacatgcgaaaattgcatcttacagtgggaagaaactatatctcacctcttcttgcggtgtaactttgcccgaagatgttggctcactgttggaatcactccgccccgaacgttggatctagcgcttgcagtgctacatattagagcaagactgacattgtcgtggagaatggaaggtattataaccatgcatgtcatggtacatatggaaatgtcaaaataactggatattcgaaaacataccgccaacggtggcggtctgcaaggaaatgttcaagattgaaatgaactacatatgcaaccaggttaagcttgagacaggagataaaagtagaaactggatacaacactttgtaacatagcgacactctctccttcctttcttttaccttagaactatacatagaatctacattcaataaaaatacagtaggtacgccgagagatttaaaatgagtaagctaacataccaactaaggccctgtttgtttcggattataatctctccagattatataatccagcgtaaataattcagcagataaacaaacacctaaattatatgttcagattatataatctatagcggagattatgataatctcgtaatctcctaagagtagcttatttgagatttttttggcaaaagacccactacccaaggttatgtaaatagaattacaatatatgacatccttctttcttcacctcaaataaacaaacaagggtactgttgtctttatgaataatctacatttatataatctagactaacaaacaactacatatagattataatatgtctagattataatctagattatataatttaaattatagtccatattatataatctataagctaaaacaaataggccctaattattagctattagttgttagctatttaaatctaagctaaaaccaactaatagcttattagttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataacgcgccacctccgctttagttctgcatacagccaacccaacacacacccgagcatatcacagtgacagacactacacggtgagcgccagaatcgttgtcctcctcgccaccctcctatccgctgccgccgcagtcgcgtcgtcctgggaggacgacaacctccaccaccacgggggccacaagtccgggcgtagcgtgcggcggtgcgaggaccggccctggcaccagcgcccccggtgcctggagcagtgcagggaggaggagcgggagaagcggcaagagcggagcaggctagaggccgacgaccgcagcggcaagggttcgtcggaggtagagcgcgagcgagagcaggaggaggagaagcagaaggaccggcggccgtacgtgttcgaccggcgcagcttccgtcgcgtggtccggagcgagcaggggtctctgagggtgctccggccgttcgacgaggtgtccaggctcctccgcggcatccgggactaccgcgtggcggtcctagaggcgaacccgcgctcgttcgtggtgcccagccataccgtagcgcactgcatctgctacgtggtggaaggtacgttcgtccggtccctgagaccacatagatcgagggcgacgccggccgtgcacgttagctgatctgttgttgcctcgtgactaggcgagggataaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacaatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgagctgatccaaaaaagaagagaaaggtagatccaaaaaagaagagaaaggtagatccaaaaaagaagagaaaggtaggctccaccggatctagataagagctcggccgccgcgctcgccaaaacgagcaagaagcaacaagagggtggcgcgcgaccgacgtgcgtacgtagctgagcctgagtggagacgttggacgtgtatgtatatacctctctgcgtgttaactatgtacgtaagcggcaggcagtgcaataagtgtggctctgtagtatgtacgtgcggatacgatgctgtatgctactgaggcaagtcccataaataaataatgacacgtgcgtgttctataatctcttcgcttcttcagtgtccccttgcggagtttggcatccattgatgccgttacactgagaacataacggacacagtagacgaaccacttgagttcttgtatgaaattctgacccttttttttgaaggatcaggagggggaaacccctactggcctggtccttttacaatcaataaaaagaaaagaaaaagaaagaaaaaggaagagctatttctatatttgccacaaatttagccagttcgggattgaggtgtcgaattctccccgtgctctgtgaatgattagtcgcaattctctactaaattcctgagtacagcggagcacggagggatctctatttgaaaacaaccactcgtttcggcttttccagatgctccaagtcataagaatagtgatctccatagagaagggaacgttgagctggtgtttgaggttcacagatgcctcttcaaggttggaaattctgggtggggtaatgccaattgagttccagcaggctttcgcgaagttgcatctgaggaaaagatgataaagggtctcctctctttgccaaaggcagttttcgcaagagtaggaatctagctccatgtttctccttctcaacatatttctggtgtttagcctgttcttaagccacagccaatagaagactctatgcttaggctgacacttacttttccaaactagtttgtagataggatgagtttgcatctggcccatgaaagatctgtaagctttttgggacgagaatcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 33 - DNA, 15441 bp, Artificial Sequence
  Expression cassette of construct 22513
  enhancer (214)..(407): eFMV
  enhancer (414)..(706): e35S
  promoter (714)..(4707): Ubi
  gene (4708)..(6165): EPSPS
  terminator (6179)..(7178): Ubi
  promoter (7198)..(9285): pPLA2
  stem_loop (9292)..(10386): targeting exon4 of PLA2
  terminator (10397)..(11392): tPLA2
  promoter (11400)..(13526)
  gene (13527)..(14204)
  terminator (14210)..(15222)
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcaagctttggaaaaccaaaacaacgaataagcaaactgcaggaaaagtatgcagtggaaaccaacccagattcggacgataggaaagtatcaagtgaatgatttgccaggaaaaggagaggggtaaaaaggggcgaagatttagaagatctaaagcacaagaaccagagattagattgaacaatagggaacttggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaagctataaagaatacattaacattcagtgaaacagcatgctgtcttcttctttttttatgcacaacagagcatacatatataccttcccaggctgaggacttggcggaggagagccgcggataggttggcggtgcagacggtctggacgggcccgaagacggagacgaacagcgggcccttcctgcccaggcaccacgcttggaacgccaagcacgcgccaaccgcggccccgccgaggacgacgatccccgcgacaagcgtggcgtcgatcctgggcgaccccaagccgaggaacctcccttccaggacgagccgtaggacggcggtgagagaggcacccgtcgcggaggtggcgcagcacaaggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggtaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctctgaacgcacacaccgcccggtctccgatctccgatccccgatcccctcgtcgatcctaggtacggcgaccatcctaccccccccccccccccctctctctctgccttctctagatcggcgatccgatccatgcttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatccagatcttcgtaaaccatggctctggtggctaggccagtgctgtctgctagagtggctgcttctaggccaagggtggccgctagaaaggctgtgagggtgagcgctgccagtcacggcgctagctctaggccagccaccgctaggaagtccagcggcctgagcggcaccgtgcgcatcccaggcgacaagagcatcagtcaccgcagcttcatgttcggcggcctggccagcggcgagactaggatcaccggcctgctcgagggcgaggacgtgatcaacaccggcaaggccatgcaggctatgggcgctcgcatccgcaaggagggcgacacctggatcatcgacggcgtgggtaatggcggcctgctggccccagaggccccactggacttcggcaacgccgccaccggctgcaggctgactatgggcctggtgggcgtgtacgacttcgacagcacctttatcggcgacgccagcctgaccaagaggccgatgggcagggtgctgaacccgctgcgcgagatgggcgtgcaggtcaagagcgaggacggcgacaggctgccggtgaccctgaggggcccaaagaccccgaccccgatcacctaccgcgtgccaatggccagcgcccaggtgaagtcagccgtgctgctggccggcctgaacaccccaggcatcaccaccgtgatcgagccgatcatgacccgcgatcacaccgagaagatgctgcagggcttcggcgccaacctgaccgtcgagactgacgctgacggcgtgaggaccatcaggctcgagggcaggggcaagctgaccggccaggtgatcgacgtgccgggcgacccaagcagcaccgccttcccactggtggccgccctgctggtgccaggctctgacgtgaccatcctgaacgtgctgatgaacccgagccgcaccggcctgatcctgaccctgcaggagatgggcgccgatatcgaggtgatcaacccgaggctggctggcggcgaggacgtcgccgacctgagggtgaggtccagcaccctgaagggcgtgaccgtgccagaggacagggccccgagtatgattgacgagtacccgatcctggccgtggccgctgccttcgccgagggcgccaccgtgatgaacggcctcgaggagctgcgcgtgaaggagagcgacaggctgagcgctgtggccaacggcctgaagctgaacggcgtggactgcgacgagggcgagactagcctggtggtgaggggcaggccagacggcaagggcctgggcaacgcttccggcgctgccgtggccacccacctggatcacaggatcgccatgtcgttcctcgtgatgggcctcgtgagcgagaacccggtgaccgtggacgacgccaccatgatcgccaccagcttccccgagttcatggacctgatggccggcctgggcgctaagatcgagctgagcgacaccaaggccgcctgatcatctagagctcgccaaggttcaattaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctcccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattactaattagctaacggaccctatttgtactcattccatgtctcataaactttgggcaccatccatccaacacatccaatctaaacacaccaaacgatggggaatggaaagagcagtattcgattcaacaatggcaaacaaatatcactgaattagaccaagaataaacctaattagacaacgacctcccaaccatcattcgtcaggctgtaaagaagataaagctgccttggggcatggatcaagcagaacaccagagatgaatccaaacacacagaaaatcacgcgcgctgtctacaatgacaacaagccccacatttcattgcagtacactgggctacaaaggcacgtacaacaaagagctagggaaacattgcggagggcacgagagagcagctaacttgacaatatagcagactgagcttgcactgttagcaggcgaggaagggaatcatggggacggagaatggggtccatgcccgcgaaggagaaggcggacgccgccacggtggcaccggcgcacgcgcacacagggaacccgcacaggcagccaaggatgctgcctcgccattgcgccggtcgtctctgccacgctcctctctctctcccgctgcatcgccgtggatggggcaagcagagagcagggactgcgacgatctgggcggaggactcgccttggagagcgcggacgcagacgggattctagggagagagcgaagacggggcgcgcgcggcgctcgcgcggcgtggtggcggcgagattagcgggggtggggggagggcggagccgtggtgagggtgtggacgccctccttaccctcttaagtagtagtagagatataatccgttccaaaatatccatccgttcaatttatatttcgtttgatctttttaccctaaatttgattgactcatcttattaaaaaagttcataactattattaatctttattgagatatcatttagcatataatatactttaagtgtggttttagattttttttaaaaaaaaaaattcgcaaaaattaaatgaaacgacccaatcaaacttgaaaagtaaaactaattataaatttgaacggaaggagtaagaggatgtttgaatgtactagagctaatagttggttgctttaaaatttgctagtagaattagctagctaataaatatctagataactattagctaatttgctaaaacagctaatagttgaactattagctagattgtttggatgtattcggctaattttaatggctaactattagctatagtacaatattcaaacacctcctaattaaaatggacaaatatctcttcttttggtcccttgcgttagatttttcatatctccttatttagtataaaagaatcatcaaaaagtggacaacccctagtggaacaccattttagtagtggttgcatgaaacctttcgcgcaccagtttctatgtgtcactctaaaaatgggacagcatgtacgtagtgcctatatatatacaagtcatctatcgttgcctcctcagttcatcactaatcacacttattgtgccctcgacgagtatctatagctagctcattaatcgattcgggggtgtgttgtcgaaggcggcattggcgagctactcgtcgcggcgtccaagcaatacctgtagcacgaaggcgatcgccgggagcgtggtcggcgagcccgtcgtgctggggcagagggtgacggtgctgacggtggacggcggcggcgtccggggtctcatcccgggaaccatcctcgccttcctggaggccaggctgcaggagctggacgcaccggaggcgaggctggcggactacttcgactacatcgccggaaccagcaccggcggtctcatcaccgccttgctgaccgcgcccggcaaggacaagcggcctctctaggctgccaaggacatcaaccacttttacatccataactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcgaaaaccccatggagatctttgcgatggcgcagatcaccaaaaagatgcttgccaacaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcgtcgactcgggagcatgccctttgtgcttatgcctccgttctgccttctgacgaatttggtactggaagcagatgagttttggttcactatcattctgaatttacacctgcgcttgctgtcagactaggcaaccaagtgacttttgtgactttgatcatgttcagtgtgtttccaagtcctaatcaatcaaaaagaaaaacagtttgttaacgattgtttgccatgtctatataataaagttgcttttatagtagcttagaattcaatcggccaactttatctcgtacgctgacagtaaaggtacatttaaaaggtgacaatggatagtctaatacttgaactgacaatagagacacattacatgtcagttgattaagtttgtaacagaaaaataaacaatactacataattgcaaagtttctttgatgtctttctttcaagaaacacaaatatatcaatgctacagtattgctgatgaatttatccatgttgagatgtttttctggtttctgatctgatcagtctcaattggtgtgctgtttcattttcatttgctgatgatcgtccgagtagttaattcttactaatatttagataatttggcatacaagcgaatcacgtagaacatgatacttttgaatgaatttatcaaagttttatcacttggtgagttgtttcatggttttcctactgatgtctcttcttcagatttctcgaggcggagccaccggcagataccccaccgggagcactgccgcgccgtgtagaggccctgctcggacgtcgaccccgtcccgatggacagcaccaggaacctgcggcagttcgacggcttcactgggtacagctcctcggccttgtccttgctggcaagcatctttttggtgatctgcgtcatcgcaaggatccatgggacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggactaccgtgaatgactatagaaccaatcatactagtataaaatatatgatgacactacgggagagacaaactttgtctggcgctaaatattttgccgagtgtgaattcacgggcactaggcaaagatcttctttgccgagtgttacgctgggcaaagtaagacactaggtaaatcagtcatttgccgagtgtccgccactaggcaaagcaaaacactggcaaatcaaaagtttacctagtgccagacactaggcaaaaaaaaaacgctcggcaaatcggaagtttccctagtgccagacactagacaaagaaaaacacttgataaactagcgtcgtcagctaacaccatccaccaaccgttaacgttgccgagtatctgacttcgacactcggcaaagaaggtctctttgcctagtgtcggtctggaacactaggcaaagaggcactttacctagtgtcgtattttgacactcagtaaaataattttttttctttctgcttccaaactttttatgatgtgttcctatagcacctagaactacatgtcaagttttggtaaaatttttgaagtttttgctatatttacttaatttattttatttaattgaatttcttttgataattcaaatttgaactcggcaaggtaagaagcgagggtagcctggaaacacactttgcctagtgttacactcggtacaggagcctcccctgcctagtgctgcactcgacaaaagattcgcctttgcctagcgctgcactcggcacaggagtcgcctttgcctagtgctgcactaggcaaagcctccgttaccgtgccttccatcgtcggacccgaaagtagcaaacaacaggttcatgtgcactataaaaagacaaaattctcgagtttcatcttttattccacataagccttatattttccattttcatatgatttttagtttaagtttgtgtcttaactttttcgttaatacgtaattctatgcattatggatgcgtgaagtatttttgtttaaaaaaatgaaatgtcaaaatacgttttgtgatctatttccatgttttcacctaacaggtggtttttactatatattctgccataactctagccttagatgtaaatcgaaaaaaaatgagagatgagctggagatagccttagatgaagcgtctgaaatataaaagaaagagtaatgttgaacgcagtaggtgtagcagctgtagttccatctctaggaaagggaactgcaatccgggctccgggcctcgcgcaatctggcctgtcgtgtagatgcagccctgtccatgacggcccaagcaacgcccgcggctctcgatccaccacggaacccactccgacacacactgacacacacatgctggatgtggatgtgctgtccaattattagtagcaattcggtaggcacaggcacgtactggccggtgttttagctgtaagtaccgaaccaatcacggttaagaaccgattaatccgtgcccagccgccgagtgcgttcgtacgtgcatcggatgcactgcatgaattgagagcatcatcatatcatacgcaggagtagtacgacgccgctgctgtcttgtccggctaatgctttgctcacagattagtccatcgcccacggtcggtgtggtgtggatcgctgatgccactgctttttgtttggtttttattcccctgataatcctccgcgtccctgaatgtatctatttattttcattccgaaatccctttcacgaaaaagaaaacgaataaaaagagagttacgaatacgcttccggcggcccacatcaccttccagcgaacatcgcgccgcgctgacgtgtcgcccatcgcggccgtccatatcgccatccgacgaccgtggaagctggcagcggccgctccgttccgtcgaaggggcaggtcagtcaggtcacccacacggccacacccgcgcgggggatacgcggtggaaaacccggcgaccacatcaaaacacgaggcgtctcccgcaggactggtcactcggcacgcaggcagaggcagcacagcagcagccagctccatccatcctctttcccctcctcgcttcgcttcctcggcggattcctcctccctcggccgtccccgtccccttcttcgccgcgccagctcgcccgagttggtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagaccagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttcgatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcaggcaaacccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggagctcgcatcatgatcatgcatcatggactcggcctactactgtggatttgtatgccattatagacttggtgctgtgaaagactgcttgatgatttgcgggtttgttgctgtgtaaaaaaaggtcccttggctcccagaagaccatgaaggttcggatctatcatgtaattccttgttatctgccaattatgtatggactatggacatgtgttgcgctgttcaacttactactacaaataagtaatcgatatgttcccttcccatgtctcggtgacaattgtctggagaagcttaggggtcgtttgtttgggattatgtctggagaaacttattttaaactaagtgtgagttcaagttaagttagattatataatctaggcagattataattccaagcgaacaggtccttagtgtttttggaaaatcctaggtgttcttttggctacattgttgtgtgtgcagatcccttgttggtctgtaagcgtggggaagtaagaatcgtccgtttctactgaagacctgctcgagttaggcaccgaggatgccggtaaccaaacagagcaatagtgtctctgtgggcacagtggagtgtgaatctgtgtgatgcaaatccgtcatttgtttagcaaaatttccagcgttgcatgatgcagtttctttaacacggacttaagggaagggaaaaaaatgttgagccaggagatccttcaatgtgttagactgacgtgatagccaactaaaccacgacgcaatgttgtcgttaatgacaaaaaaactatttgttcctaaatccttggcgacattgcatggctgtctcatgagataatggtctcatctcttatttatctcttatttatagccggaagtggtagtgacccctgcttgattgctcgtatgccatctcaagttctcaaccgtgtcgagcagccattttcccatctcaagcgcatcatcgtttcgtttgacctcatctgctatcctgctcctagtgcaaatcacatgcgacagaaagtgtgcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 34 - DNA, 10671 bp, Artificial Sequence
  Expression cassette of construct 22807
  enhancer (165)..(256): eNOS
  promoter (301)..(2097): Ubi
  gene (2114)..(6283): Cas9
  misc_feature (6218)..(6274): NLS-TAG
  terminator (6289)..(6541): tNOS
  promoter (6548)..(6922): pU3
  misc_feature (6924)..(6941)
  misc_feature (6924)..(7026): sgRNA
  promoter (7037)..(9028): Ubi
  gene (9045)..(10223): PMI
  terminator (10246)..(10498): tNOS
cctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagtcaacgtggagacaggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 35 - DNA, 14475 bp, Artificial Sequence
  Expression cassette of construct 22808
  enhancer (165)..(256): eNOS
  promoter (301)..(2097): Ubi
  gene (2116)..(5190): TNFw
  terminator (2116)..(5190): tNOS
  promoter (5483)..(7475): Ubi
  gene (7489)..(10563): TNRv
  terminator (10570)..(10822): tNOS
  promoter (10841)..(12832): Ubi
  gene (12849)..(14027): PMI
  terminator (14050)..(14302): tNOS
cctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgcgccaccatgggaaaacctattcctaatcctctgctgggcctggattctaccggaggcatggcccctaagaaaaagcggaaggtggacggcggagtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgattaaaagaacaaatagaagaatccccgagcggacctctcacagagtggccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaagtgtacggctacagaggaaagcatctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccaaaccaggaacaagcatatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaaccctagtcgaagacaaccggtgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagcggccgcgccaccatgggaaaacctattcctaatcctctgctgggcctggattctaccggaggcatggcccctaagaaaaagcggaaggtggacggcggagtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacgggctgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagccacgacggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgattaaaagaacaaatagaagaatccccgagcggacctctcacagagtggccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaagtgtacggctacagaggaaagcatctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccaaaccaggaacaagcatatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagacggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 36 - DNA, 10668 bp, Artificial Sequence
  Expression cassette of construct 22873
  enhancer (165)..(256): eNOS
  promoter (301)..(2097): Ubi
  gene (2114)..(6280): Cas9m
  misc_feature (6215)..(6271): NLS-TAG
  terminator (6286)..(6538): tNOS
  promoter (6545)..(6919): pU3
  misc_feature (6921)..(6938)
  misc_feature (6921)..(7023): sgRNA
  promoter (7034)..(9025): Ubi
  gene (9042)..(10220): PMI
  terminator (10243)..(10495): tNOS
cctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctaaaatggataagaagtactctattggcctcgatattggaaccaactctgtgggctgggccgtgatcaccgatgagtacaaggtgccatctaagaagttcaaggtgctcggcaacaccgataggcactctatcaagaagaacctcatcggtgccctcctcttcgattctggcgagaccgccgaggccaccaggctcaagaggaccgccaggaggaggtacaccaggaggaagaacaggatctgctacctccaggagatcttctctaacgagatggccaaggtggatgattctttcttccacaggctcgaggagtctttcctcgtggaggaggataagaagcacgagaggcacccaatcttcggcaacatcgtggatgaggtggcctaccacgagaagtacccaaccatctaccacctcaggaagaagctcgtggattctaccgataaggccgatctcaggctcatctacctcgccctcgcccacatgatcaagttcaggggccacttcctcatcgagggcgatctcaacccagataactctgatgtggataagctcttcatccagctcgtgcagacctacaaccagctcttcgaggagaacccaatcaacgcctctggcgtggatgccaaggccatcctctctgccaggctctctaagtctaggaggctcgagaacctcatcgcccagctcccaggcgagaagaagaacggcctcttcggcaacctcatcgccctctctctcggcctcaccccaaacttcaagtctaacttcgatctcgccgaggatgccaagctccagctctctaaggatacctacgatgatgatctcgataacctcctcgcccagatcggcgatcagtacgccgatctcttcctcgccgccaagaacctctctgatgccatcctcctctctgatatcctcagggtgaacaccgagatcaccaaggccccactctctgcctctatgatcaagaggtacgatgagcaccaccaggatctcaccctcctcaaggccctcgtgaggcagcagctcccagagaagtacaaggagatcttcttcgatcagtctaagaacggctacgccggctacatcgatggcggcgcctctcaggaggagttctacaagttcatcaagccaatcctcgagaagatggatggcaccgaggagctcctcgtgaagctcaacagggaggatctcctcaggaagcagaggaccttcgataacggctctatcccacaccagatccacctcggcgagctccacgccatcctcaggaggcaggaggatttctacccattcctcaaggataacagggagaagatcgagaagatcctcaccttccgcatcccatactacgtgggcccactcgccaggggcaactctaggttcgcctggatgaccaggaagtctgaggagaccatcaccccttggaacttcgaggaggtggtggataagggcgcctctgcccagtctttcatcgagaggatgaccaacttcgataagaacctcccaaacgagaaggtgctcccaaagcactctctcctctacgagtacttcaccgtgtacaacgagctcaccaaggtgaagtacgtgaccgagggcatgaggaagccagccttcctctctggcgagcagaagaaggccatcgtggatctcctcttcaagaccaacaggaaggtgaccgtgaagcagctcaaggaggattacttcaagaagatcgagtgcttcgattctgtggagatctctggcgtggaggataggttcaacgcctctctcggcacctaccacgatctcctcaagatcatcaaggataaggatttcctcgataacgaggagaacgaggatatcctcgaggatatcgtgctcaccctcaccctcttcgaggatagggagatgatcgaggagaggctcaagacctacgcccacctcttcgatgataaggtgatgaagcagctcaagaggaggaggtacaccggctggggcaggctctctaggaagctcatcaacggcatcagggataagcagtctggcaagaccatcctcgatttcctcaagtctgatggcttcgccaacaggaacttcatgcagctcatccacgatgattctctcaccttcaaggaggatatccagaaggctcaggtgtctggccagggccactctctccacgagcagatcgccaacctcgccggctctccagccatcaagaagggcatcctccagaccgtgaaggtggtggatgagctcgtgaaggtgatgggccacaagccagagaacatcgtgatcgagatggccagggagaaccagaccacccagaagggccagaagaactctagggagaggatgaagaggatcgaggagggcatcaaggagctcggctctcagatcctcaaggagcacccagtggagaacacccagctccagaacgagaagctctacctctactacctccagaacggcagggatatgtacgtggatcaggagctcgatatcaacaggctctctgattacgatgtggatcacatcgtgccacagtctttcctcaaggatgattctatcgataacaaggtgctcaccaggtctgataagaacaggggcaagtctgataacgtgccatctgaggaggtggttaagaagatgaagaactactggaggcagctcctcaacgccaagctcatcacccagaggaagttcgataacctcaccaaggccgagaggggcggcctctctgagctcgataaggccggcttcatcaagaggcagctcgtggagaccaggcagatcactaagcacgtggcccagatcctcgattctaggatgaacaccaagtacgatgagaacgataagctcatcagggaggtgaaggtgatcaccctcaagtctaagctcgtgtctgatttcaggaaggatttccagttctacaaggtgagggagatcaacaactaccaccacgcccacgatgcctacctcaacgccgtggtgggcaccgccctcatcaagaagtacccaaaactcgagtctgagttcgtgtacggcgattacaaggtgtacgatgtgaggaagatgatcgccaagtctgagcaggagatcggcaaggccaccgccaagtacttcttctactctaacatcatgaacttcttcaagaccgagatcaccctcgccaacggcgagatcaggaagaggccactcatcgagaccaacggcgagaccggcgagatcgtgtgggataagggcagggatttcgccaccgtgaggaaggtgctctctatgccacaggtgaacatcgtgaagaagaccgaggtgcagaccggcggcttctctaaggagtctatcctcccaaagaggaactctgataagctcatcgccaggaagaaggattgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagtcaacgtggagacaggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 37 - DNA, 10673 bp, Artificial Sequence
  Expression cassette of construct 23123
  enhancer (165)..(256): eNOS
  promoter (301)..(2097): Ubi
  gene (2114)..(6283): Cas9
  misc_feature (6218)..(6274): NLS-TAG
  terminator (6289)..(6541): tNOS
  promoter (6548)..(6922): pU3
  misc_feature (6924)..(6943)
  misc_feature (6924)..(7028): sgRNA
  promoter (7039)..(9030): Ubi
  gene (9047)..(10225): PMI
  terminator (10248)..(10500): tNOS
cctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagggtcaacgtggagacaggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 38 - DNA, 10693 bp, Artificial Sequence
  Expression cassette of construct 23501, gRNA target 1 dual guides
  promoter (214)..(734): 35S
  gene (748)..(4917): Cas9
  misc_feature (4852)..(4908): NLS-TAG
  terminator (4923)..(5175): tNOS
  promoter (5196)..(5570): pU3
  misc_feature (5572)..(5591)
  misc_feature (5592)..(5636): crRNA
  promoter (5643)..(6158): pU6
  misc_feature (6159)..(6246): tracrRNA
  promoter (6253)..(8245): Ubi
  gene (8257)..(9435): PMI
  terminator (9440)..(10474): Ubi
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccgagtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaccggaattcgggacccgggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcatgcagtcgaagtaatcggcggttttagagctatgctgttttgaatggtcccaaaactttttttttactagttttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggaaccattcaaaacagcatagcaagttaaaataaggctagtccgttatcaacttgaaaaagtggcaccgagtcggtgctttttttttcctaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgactaaatagatgcagaagctgatcaacagcgtgcagaactacgcctggggcagcaagaccgccctgaccgagctgtacggcatggagaaccccagcagccagcccatggccgagctgtggatgggcgcccaccccaagagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagaccactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgatagctacgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 39 - DNA, 10264 bp, Artificial Sequence
  Expression cassette of construct 23501, gRNA target 1 single guide
  promoter (214)..(734): 35S
  gene (748)..(4917): Cas9
  misc_feature (4852)..(4908): NLS-TAG
  terminator (4923)..(5175): tNOS
  promoter (5196)..(5711): pU6
  misc_feature (5712)..(5817): sgRNA
  misc_feature (5713)..(5732)
  promoter (5824)..(7816): Ubi promoter
  gene (7828)..(9006): PMI
  terminator (9011)..(10045): Ubi terminator
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccgagtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaccggaattcgggaccctttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgtgcagtcgaagtaatcggcggttttagagctagaaatagcaagttaaaataaggctagtccgttatcaacttgaaaaagtggcaccgagtcggtgctttttttttcctaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgactaaatagatgcagaagctgatcaacagcgtgcagaactacgcctggggcagcaagaccgccctgaccgagctgtacggcatggagaaccccagcagccagcccatggccgagctgtggatgggcgcccaccccaagagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagaccactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgatagctacgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 40 - DNA, 10692 bp, Artificial Sequence
  Expression cassette of construct 23501, gRNA target 2 dual guides
  promoter (214)..(734): 35S
  gene (748)..(4917): Cas9
  misc_feature (4852)..(4908): NLS-TAG
  terminator (4923)..(5175): tNOS
  promoter (5196)..(5570): pU3
  misc_feature (5572)..(5590)
  misc_feature (5591)..(5635): crRNA
  promoter (5642)..(6157): pU6
  misc_feature (6158)..(6245): tracrRNA
  promoter (6252)..(8244): Ubi promoter
  gene (8256)..(9434): PMI
  terminator (9439)..(10473): Ubi terminator
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccgagtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaccggaattcgggacccgggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagagaccggcaggtacgtcggttttagagctatgctgttttgaatggtcccaaaactttttttttactagttttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggaaccattcaaaacagcatagcaagttaaaataaggctagtccgttatcaacttgaaaaagtggcaccgagtcggtgctttttttttcctaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgactaaatagatgcagaagctgatcaacagcgtgcagaactacgcctggggcagcaagaccgccctgaccgagctgtacggcatggagaaccccagcagccagcccatggccgagctgtggatgggcgcccaccccaagagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagaccactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgatagctacgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 41 - DNA, 10262 bp, Artificial Sequence
  Expression cassette of construct 23501, gRNA target 2 single guide
  promoter (214)..(734): 35S promoter
  gene (748)..(4917): Cas9
  terminator (4923)..(5175): tNOS
  promoter (5196)..(5711)
  misc_feature (5712)..(5730)
  misc_feature (5712)..(5815): sgRNA
  promoter (5822)..(7814): Ubi promoter
  gene (7826)..(9004): PMI
  terminator (9009)..(10043): Ubi terminator
cctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccgagtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaccggaattcgggaccctttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgagaccggcaggtacgtcggttttagagctagaaatagcaagttaaaataaggctagtccgttatcaacttgaaaaagtggcaccgagtcggtgctttttttttcctaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgactaaatagatgcagaagctgatcaacagcgtgcagaactacgcctggggcagcaagaccgccctgaccgagctgtacggcatggagaaccccagcagccagcccatggccgagctgtggatgggcgcccaccccaagagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagaccactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgatagctacgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcag

SEQ ID NO: 42 - DNA, 1366 bp, Artificial Sequence
  TALEN-induced mutation in Event 38A ID 22808-4108 allele 2
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacaggacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 43 - DNA, 1366 bp, Artificial Sequence; identical to SEQ ID NO: 10
  CRISPR-induced MTL mutation in Event 27A ID 22807-4073 allele 2

SEQ ID NO: 44 - DNA, 1363 bp, Artificial Sequence; identical to SEQ ID NO: 13
  CRISPR-induced MTL mutation in Event 27A ID 22807-4081 allele 2

SEQ ID NO: 45 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctatagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 46 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagctcgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 47 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcctgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 48 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttacgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 49 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatacggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 50 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgattggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 51 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggaaaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 52 - protein, 401 aa, Zea mays
MASYSSRRPCNTYSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 53 - protein, 401 aa, Zea mays
MASYSSRRPCNTCSTKAMAGSVVGELVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 54 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCLRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 55 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVTMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 56 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENIRTLVGIGERMLAQRVSRVNVETGRYEPVTGEGSNADALGGLARQLSEERRTRLARRVSAINPRGSRCASYDI

SEQ ID NO: 57 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVIGEGSNADALGGLARQLSEERRTRLARRVSAINPRGSRCASYDI

SEQ ID NO: 58 - protein, 401 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMENCPRIFPQKSRLAAAMSALRKPKYNGKCMRSLIRSILGETRAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERKTRLARRVSAINPRGSRCASYDI

SEQ ID NO: 59 - protein, 379 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVENR

SEQ ID NO: 60 - protein, 396 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRTGDWRRKQCRCPRWAR

SEQ ID NO: 61 - protein, 379 aa, Zea mays; identical to SEQ ID NO: 59

SEQ ID NO: 62 - protein, 394 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETTGDWRRKQCRCPRWAR

SEQ ID NO: 63 - protein, 395 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGTGDWRRKQCRCPRWAR

SEQ ID NO: 64 - protein, 393 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETTGDWRRKQCRCPRWA

SEQ ID NO: 65 - protein, 395 aa, Zea mays; identical to SEQ ID NO: 63

SEQ ID NO: 66 - protein, 398 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETREVRTGDWRRKQCRCPRWAR

SEQ ID NO: 67 - protein, 398 aa, Zea mays; identical to SEQ ID NO: 66

SEQ ID NO: 68 - protein, 384 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETKGGTNR

SEQ ID NO: 69 - protein, 383 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGGTNR

SEQ ID NO: 70 - protein, 396 aa, Zea mays; identical to SEQ ID NO: 60

SEQ ID NO: 71 - protein, 396 aa, Zea mays; identical to SEQ ID NO: 60

SEQ ID NO: 72 - protein, 395 aa, Zea mays; identical to SEQ ID NO: 63

SEQ ID NO: 73 - protein, 437 aa, Sorghum bicolor
MATYYSSRRPCNACSTKAMAGSVVGEPVVLGQRVTVLTVDGGGIRGLIPGTILAFLEARLQELDGPEVRLADYFDYIAGTSTGGLITAMLTAPGKDRRPLYAAKDINQFYMENCPRIFPQKSSRLAAAMSALRKPRYNGKCLRNLIMSMLGETRVSDTLTNVIIPTFDVRLLQPIIFSTYDAKSMPLKNALLSDVCIGTSAAPTYLPAHYFQTKDAGSGKEREYNLIDGGVAANNPTMVAMTQITKKMLASKEKAEELYPVKPWNCRKFLVLSIGTGSTSEQGLYTARQCSRWGICRWIRNNGMAPIIDIFMAASSDLVDIHVAAMFQSLHSDGDYLRIQDNSLHGAAATVDAATPENMRTLVGIGERMLAQRVSRVNVETGRYEPVPGEGSNADALAGIARQLSEERRTRLARRTSAIVSSGGASRRTCASKVSNV

SEQ ID NO: 74 - protein, 421 aa, Setaria italica
MASYSSRRPCNACRTKAMAGSVVGEPVVPGQRVTVLTIDGGGIRGLIPGTILAFLEARLQELDGPEARLADYFDCIAGTSTGGLITAMITTPGEDKRPLFAARDINRFYFDNCPRIFPQSRSSLAAAMSALRKPRYNGKYLRSTIRSMLGETRVSDALTNVVIPTFDIKLIQPIIFSTYDVKNMPLKNALLSDVCISTSAAPTYLPAHYFQIQDAGGKTREYNLIDGGVAANNPTMVAMTQITKMMLAKDKEELYPVKPEDCRKFLVLSIGTGSTSDEGLFTARQCSRWGVVRWLRNNGMAPIIDIFMAASSDLVDIHAAVLFQSLHSDGHSLRGAAATVDAATPENMRTLVGIGERMLAQRVSRVNVETGRYEPVPGEGSNADALVALARQLSDERRARIARRAAAACAGGSRCCSPVKT

SEQ ID NO: 75 - protein, 422 aa, Hordeum vulgare
MASYWCRRPCESCSTRAMAGSVVGQPVAPGQRVTVLTIDGGGIRGLIPGTILAFLEARLQELDGPDARLADYFDCIAGTSTGGLITAMLTAPGQDGRPLFAAKDVNRFYLDNGPYIFPQRRCALAAVTASLRRPRYSGKYLHGKIRSMLGETRLCDALTDVVIPTFDVKLLQPIIFSTYDARNMPLKNARLADICIGTSAAPTYLPAHHFHTQDDNGKEREYNLIDGGVAANNPTMVTMTQITKKMMVKDREELYPVKPSDCGKFLVLSIGTGSTSDQGLYTAKQCSQWGIIRWLRNKGMAPIIDIFMAASSDLVDIHAAVLFQSLHSDGNYLRIQDNSLHGPAATVDAATPENMAELLRIGERMLAQRVSRVNVETGRYEEIRGAGSNADALAGFAKQLSDERRTRLGRRRVGAGRLKSRR

SEQ ID NO: 76 - protein, 423 aa, Brachypodium distachyon
MASYACRRPCESCRTRAMAGGVVGEPTTPGQRVTVLTIDGGGIRGLIPGTILAFLEDRLQELDGPDARLADYFDCIAGTSTGGLITAMITAPGEEGRPLFAAEDINRFYLDNGPQIFPQKRSSLMSVLASLTRPRYNGKFLHGKIRSMLGETRVCDTLTDVVIPTFDVRLLQPIIFSTYDAKSMPLKNALLSDVCISTSAAPTFLPAHYFQTEDDNGKVREYNLIDGGVAANNPTMVAMTQITKKIMAKDKEELYPVKPSDCGKFLVLSIGTGSTSDQGLYTAKQCSRWGIIRWLRNKGMAPIIDIFMAASSDLVDIHAAVLFQSLHSDGDCYLRIQDNSLRGAAATVDTATPDNMRELVRIGERMLAQRVSKVNVETGRYEEMQGAGTNADALAGFARQLSDERRARFGPRDGAPANGKSRC

SEQ ID NO: 77 - protein, 414 aa, Oryza sativa
MAGCVVGEPASAPGQRVTLLAIDGGGIRGLIPGTILAFLEARLQELDGPDARLADYFDCIAGTSTGGLITAMLAAPGDHGRPLFAASDINRFYLDNGPRIFPQKRCGMAAAMAALTRPRYNGKYLQGKIRKMLGETRVRDTLTNVVIPTFDVRLLQPTIFSTYDAKSMPLKNALLSDICISTSAAPTYLPAHCFQTTDDATGKVREFDLIDGGVAANNPTMVAMTQITKKIMVKDKEELYPVKPSDCGKFLVLSLGTGSTSDQGMYTARQCSRWGIVRWLRNKGMAPIIDIFMAASSDLVDIHAAVMFQSLHSDGDYLRIQDNTLHGDAATVDAATRDNMRALVGIGERMLAQRVSRVNVETGRYVEVPGAGSNADALRGFARQLSEERRARLGRRNACGGGGEGEPSGVACKR

SEQ ID NO: 78 - protein, 275 aa, Triticum aestivum
MLGETRLSDALTDVVIPTFDVKLLQPIIFSTYDAKSMPLKNARLADVCIGTSAAPTYLPAHHFHTHDGNGKEREYNLIDGGVAANNPTMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEVKGAGNNADALAGFARQLSDERRTRLGSRRGGAGRLKSSR

SEQ ID NO: 79 - protein, 416 aa, Musa acuminata
MASDQTPPATADAPISTPPPSFGKRVTVLCIDGGGVRGLIPATIIAFLEAELQKLDGPEARIADYFDVIAGTSTGGLVTAMLTAPNKEKRPLFAAKDIVQFYLDNSPKIFPQKNAGLFNSALNLVGAVSGPKYDGKYLHAIIRQLLGDTRLSQTLTNVVIPTFDIKFLQPTIFSTYQTKSTPLKDALLSDICIGTSAAPTYLPGHYFETKDDNGNKRSFNLVDGGVAANNPTLTAMTEVSKEILLSNPDFFSYQPVEYDRFLVISLGTGAPKQEEKFTAQESAKWGVLGWLLNKGTTPLIDIFTQASADMVDIHASVLFQALNKGKNYLRIEDDTLTGQTSSVDVSTKKNLQDLVDIGNSLLKKPVSRVNIETGHSEAVDGEGTNEAALTGFAKKLSDEKRRRQSKQLTSSDATQH

SEQ ID NO: 80 - protein, 402 aa, Elaeis guineensis
MGSNESPVANPPPCKGKVVTILSIDGGGVRGIIPGTILEFLEAKLQELDGPDARIADYFDIIAGTSTGGLVTTMLTAPNKDNRPLFSAKDIIQFYLENCPKIFPQRTGLLAGALNLFGAVSGPKYDGKFLHSKVKELLGDTKLHQTLTNIVIPAFDIKLLQPVIFSTFETKTDPSKDALLSDICISTSAAPTYLPAHYFETKDSQGKTRSFNLVDGGVAANNPMLIATSQITKQIFWNNEDFSKFKPTDFAKFLVVSLGTGSPKQEQKFTAPESAKWGLLGWLQNKGSTPIIDIFSQSSADMVDIHASILFQALRSEKNYLRIQDDTLEGDTASVDVSTSENLRKLVQVGQDLLKKPVSRVNLETGVSEACDVEGTNEDALIRFAKMLSNERKSRNAKMSAA

SEQ ID NO: 81 - protein, 407 aa, Arabidopsis thaliana
MQMDSPKSPLQPPTYGNLVTILSIDGGGIRGLIPAVILGFLESELQKLDGEEARLADYFDVIAGTSTGGLVTAMLTAPNKEGRPLFAASEIKDFYLEQCPKIFPQDHFPFSAAKKLVKSLTGPKYDGKYLHQLIHAKLGDTKLSQTLTNVVIPTFDIKHLQPTIFSSYEVKNHPLKDAALADIAISTSAAPTYLPAHFFKVEDLNGNAKEYNLIDGGVAANNPALLAIGEVTNEISGGSSDFFPIRPNDYGRFLVLSLGTGNHKAEEKFNAKEVAGWGLLNWLTHDNSTPIIDAFSQASSDMVDFHLSAVFRALHSEANYIRIQDDTLTGDAASVDIATVENLDILAKTGDELLKKPVARVNLDSGCNENAYETTNEHALIKLAGILSKEKKIRDIRSPHAKAPIRI

(19) United States
(12) Patent Application Publication
(10) Pub. No.: US 2018/0332790 A1
(43) Pub. Date: Nov. 22, 2018

(54) HAPLOID INDUCTION COMPOSITIONS AND METHODS FOR USE THEREFOR

(71) Applicant: Syngenta Participations AG, Basel (CH)

(72) Inventors: Timothy KELLIHER, Durham, NC (US); Brent DELZER, Janesville, WI (US); Satya CHINTAMANANI, Slater, IA (US); David Stewart SKIBBE, Durham, NC (US); Zhongying CHEN, Durham, NC (US); Dakota STARR, Durham, NC (US); Sebastian Volker WENDEBORN, Stein (DE); Mark LEDSON, Greensboro, NC (US); Jeffrey David FOWLER, Greensboro, NC (US)

(73) Assignee: Syngenta Participations AG, Basel (CH)

(21) Appl. No.: 15/776,957

(22) PCT Filed: Nov. 17, 2016
(86) PCT No.: PCT/US2016/062548

§ 371 Date: May 17, 2018

Related U.S. Application Data
(60) Provisional application No. 62/256,902, filed on Nov. 18, 2015.
(60) Provisional application No. 62/300,507, filed on Feb. 26, 2016.

(51) Int. Cl. A01H 1/08

(52) U.S. Cl. CPC A01H 1/08

(57) ABSTRACT

Provided here are methods of using a mutated patatin-like phospholipase IIα (“pPLAIIα,” renamed here MATRILINEAL) to induce haploid induction in plants, cloning a pPLAIIα to induce haploid induction in plants, and genetically engineering a plant to contain a mutated pPLAIIα. Also provided are methods of applying topical and spray chemicals, lipids, and RNAi molecules to plants during pollination in order to induce haploid production. Further provided are methods of chemically treating plants during pollination to induce haploids while also reducing embryo abortion and increasing seed set.

### CLAIM FOR PRIORITY

This application claims the benefit under 35 U.S.C. § 120 of U.S. Provisional Application No. 62/256,902, filed on Nov. 18, 2015, and U.S. Provisional Application No. 62/300,507, filed on Feb. 26, 2016, the contents of which are incorporated herein by reference

### SEQUENCE LISTING

This application is accompanied by a sequence listing entitled 80906-PCT_ST25.txt, created Nov. 16, 2016, which is approximately 392 kilobytes in size. This sequence listing is incorporated herein by reference in its entirety. This sequence listing is submitted herewith via EFS-Web, and is in compliance with 37 C.F.R. § 1.824(a)(2)-(6) and (b).

### FIELD OF THE INVENTION

The presently disclosed subject matter relates to the diagnostic detection of haploid induction (“HI”) or its absence and/or presence in plants which are or are not haploid inducers. More particularly, the presently disclosed subject matter relates to nucleic acids that can be employed for inducing HI in plants and/or the biological activities which can be modified in order to produce or prevent HI in either a plant that would otherwise exhibit HI or in a plant that would otherwise not exhibit HI. Even more particularly, the presently disclosed subject matter relates to a nucleic acid molecule that encodes a biologically active molecule as well as methods for using the same to regulate HI in plants.

Provided here are a series of independent human-induced mutations found in at least one patatin-like phospholipase A2α (“PLA”) gene of maize; maize plants having these mutations in at least one of their PLA genes; and a method of creating and identifying similar and/or additional mutations in the PLA gene by screening pooled and/or individual maize plants. The maize plants of the present invention induce haploidy as a result of non-transgenic mutations in at least one of their PLA genes. Also provided are methods of inducing de novo haploid induction by chemical application to the reproductive tissues of plants. Also provided are methods of increasing the seed setting rate and haploid induction rate (“HIR”, defined herein as the number of surviving haploid kernels over the total number of kernels after an ear is pollinated with haploid inducer pollen), by chemical application to reproductive plant tissues during the pollination.

### BACKGROUND

Pollination is a complex process. Angiosperm pollen grains consist of a large vegetative cell and two male gametes (sperm cells). After landing on the stigma, the grain germinates a pollen tube that exhibits rapid tip-growth as it navigates down the female transmitting tract, guided by chemo-attractants secreted by the two synergid cells at the micropylar end of the embryo sac. During transmittance down the tube, the sperm are connected to each other and the vegetative nucleus by a stringy cytoplasm called the male germ unit. Shortly after contact with one of the two synergids, the pollen tube bursts and the two sperm are propelled across the dying synergid cell cytoplasm to independently fuse with the egg and central cells of the embryo sac, completing double fertilization. Even after initial contact, fertilization failure events can be rescued by a second pollen tube that fertilizes the embryo sac via interaction with the persistent synergid cell.

Breeders cross inbred parent lines, one acting as a male and one as a female, in order to form hybrid seed. The process of developing inbred parent lines which are substantially homozygous usually requires a hybrid cross to be selected and self-pollinated (selfed) for numerous generations to become nearly homozygous. This process is time consuming and expensive. To shorten the time to develop homozygous inbreds in maize, rice, wheat, barley, and other crops, breeders may opt to use a haploid inducer line to induce haploid seed production on a hybrid parent. The chromosomes of the haploid plants are then doubled, for example by a chromosome doubling agent such as colchicine, to form doubled haploid homozygous inbred lines.

Haploid induction (“HI”) is a class of plant phenomena characterized by loss of the inducer chromosomes during embryo development. WO2012/030893, incorporated herein by reference, describes a region of maize chromosome 1 that is may be responsible for haploid induction. The identified markers in that region increased haploid induction are described as being between 48,249,509-51,199,249, which is associated with a public marker umc1169 that has the physical position of (60,213,661). This region does not seem to align with the Haploid Induction region in Stock 6. Dong et al., (2013) Theor. Appl. Genet. 126: 1713-1720, describe a QTL located in bin 1.04 which explains up to 66% of the genotypic variance for HIR.

Haploid induction has been observed in numerous plant species, such as sorghum, barley, wheat, and other grasses. In maize, HI appears to be a result of rearrangements of, mutations in, and/or recombinations, insertion, or deletions within a region of chromosome 1 (with the notable exception of the ig type haploid induction, which is a result of a mutation in the INDETERMINATE GAMETOPHYTE1 gene on chromosome 3). Purported HI lines have been studied and roughly identified. However, experimental evidence demonstrating a causative genetic agent of HI in maize has not been presented. Nor have the markers listed herein that associate with this trait been previously identified.

In maize haploid seed or embryos are specifically produced by making crosses between a haploid inducer male (i.e., “haploid inducer pollen”) and virtually any ear that one chooses—the ear could be of any inbred, hybrid, or other germplasm. Haploids are produced when the haploid inducer pollen DNA is not fully transmitted and/or maintained through the first cell divisions of the embryos. The resulting phenotype is not fully penetrant, with some ovules containing haploid embryos, and others containing diploid embryos, aneuploid embryos, chimeric embryos, or aborted embryos. The haploid kernels have embryos that contain only the maternal DNA plus normal triploid endosperm. After haploid induction, haploid embryos or seed are typically segregated from diploid and aneuploid siblings using a phenotypic or genetic marker screen and grown or cultured into haploid plants. These plants are then converted either naturally or via chemical manipulation (i.e., colchicine) into doubled haploid (DH) plants which then produce inbred seed.

HI lines contain a quantitative trait locus (“QTL”) on Chromosome 1 responsible for at least 66% of the variation in haploid induction. The QTL causes haploid induction at different rates when it is introgressed into various backgrounds. All haploid inducer lines used in the seed industry are derivatives of the founding HI line, known as Stock6, and all have the haploid inducer chromosome 1 QTL mutation. Here, we uncover the key mutation in that QTL which, when complemented, rescues normal reproduction. While the origins of this mutation are unclear, it is in all inducer lines, including Stock6.

Plant breeding is facilitated by the use of doubled haploid (DH) plants. The production of DH plants enables plant breeders to obtain inbred lines without multi-generational inbreeding, thus decreasing the time required to produce homozygous plants. DH plants provide an invaluable tool to plant breeders, particularly for generating inbred lines, QTL mapping, cytoplasmic conversions, trait introgression, and F2 screening for high throughput trait improvement. A great deal of time is spared as homozygous lines are essentially generated on one generation, negating the need for multigenerational conventional inbreeding. In particular, because DH plants are entirely homozygous, they are very amenable to quantitative genetics studies. The production of haploid seed is critical for the doubled haploid breeding process. Haploid seed are produced on maternal germplasm when fertilized with pollen from a gynogenetic inducer, such as Stock 6.

Maize haploid inducer plants produce pollen which, when crossed onto non-inducer germplasm, results in the gynogenic development of haploid seeds. Unfortunately, this process often yields a low frequency of haploid kernels. Inefficient haploid induction frequency is a limiting factor in maize doubled haploid breeding programs.

### SUMMARY

A high HIR allows a higher frequency of haploid seeds to be formed on the parent plant of interest. The parent plants can be pre-screened with genetic markers associated with desired traits or phenotypically-observed traits to enrich the genetic potential of the parent plants. When these desired parent plants are pollinated by a haploid inducer that has a higher HIR, a higher potential of desired doubled haploids is obtained with the desired genotype and phenotype.

Although the doubled haploid process resulted in faster production of homozygous inbreds, the volume of doubled haploid inbreds that are produced may be limited. Known inducer lines, including but not limited to: Stock 6, MHI (Moldovian Haploid Inducer), indeterminate gametophyte (“ig”) mutation, KEMS, RWK, ZEM, ZMS, and KMS. All have a relatively low HIR. Stock 6, for example, only induces 1-3% haploid seeds. As such, the induction of haploids has been a rate-limiting step in the process of producing doubled haploid lines.

We have invented a way to induce haploid production and/or increase the haploid induction rate in plants by treating the plants with a lipid compound, a phospholipase inhibitor, and a fatty acid desaturase inhibitor. One such set of methods includes applying specific chemicals to reproductive tissues when crossing with wild-type (non-haploid inducer) pollen. For the first time, we have triggered de novo haploid induction chemically. This is accomplished by administering a concentration of the phospholipase inhibitor methyl alpha linolenyl fluorophosphonate (MALFP) to the flower during pollination, which leads to a high rate of haploid induction: up to 9% HIR. Separately, we have triggered de novo haploid induction by administering a concentration of arachidonyl fluorophosphonate (MAFP) to the flower during pollination. Separately, we have triggered de novo haploid induction by administering a concentration of 1,2-distearoyl-sn-glycero-3-phosphatidyl choline (also known as distearyl-phosphatidyl choline; “DSPC”) to the flower during pollination. We have also triggered de novo haploid induction by administering a concentration of alpha linolenic acid to the flower during pollination. We have also triggered de novo haploid induction by administering readily available compounds, including corn oil and linseed oil, as well as chemically-synthesized linoleic acid, oleic acid ethyl ester (OAEE), arachidonic acid methyl ester, (AAME) and the phospholipase inhibitor manoalide. We have also increased the rate of haploid induction when crossing plants with a haploid inducer line (e.g., RWK, Stock 6, or ZMS) by administering a concentration of the phospholipase inhibitor methyl alpha linolenyl fluorophosphonate (MALFP). At concentrations of 2% MALFP+surfactant blend 91 emulsified with a buffered DML solution, we have doubled the haploid induction rate in pollinations using RWK as the pollinator. The typical induction rate in RWK is about 10-18%. With MALFP applied the induction rate increases to 20-35%. We have also boosted the haploid induction rate by applying concentrations of linoleic acid (LLE), linoleic acid ethyl ester (LLAEE), and the phospholipase inhibitor called methyl arachidonyl fluorophosphonate (MAFP) to the flower during pollination. Furthermore, doing so in this manner also reduces the rate of embryo abortion and increases the rate of kernel formation for MALFP, LLAEE, MAFP, and LLE. Together these effects lead to an increase in the total number of haploid embryos recovered on the ear after pollination.

We have also cloned and characterized the gene responsible for haploid induction in maize. The gene is PLA2 and it has pollen-specific expression. The PLA2 protein appears to localize to the sperm-cell cytoplasm, perhaps the endoplasmic reticulum or golgi bodies. The identification of the gene has led to inventions of several new techniques to improve the haploid induction process, defined as the act of producing haploid embryos, kernels, seed, or plants by crossing any ear with haploid inducer pollen. The identification of the gene has also led to the inventions of new methods to induce haploids. Another set includes methods to create new haploid inducer lines by changing the sequence of the causative gene, either through targeted mutagenesis, TILLING, or CRISPR/Cas9. Expression of the PLA2 protein may be downregulated using RNAi or by using targeting mutagenesis in the promoter, 3′ UTR, 5′ UTR, or the splice sites.

Based on the identification of this mutation, we invent ways to modify and improve the haploid induction process and, for the first time, we disclose methods to produce haploids de novo via chemical treatment during pollination. We show methods to increase the haploid induction rate (“HIR,” i.e., the percentage of haploid embryos found on a given haploid induced ear) and also methods to raise the kernel survival rate during haploid induction. We also discuss methods to create new haploid inducer lines using genetic modification (“GM”) or targeted mutagenesis strategies.

The haploid induction process can be improved through a variety of methods. First, one can strive to improve the average HIR. The HIR is rate-limiting in large-scale doubled haploid (“DH”) plant production because the HIR is relatively low. See FIG. 1. With rare exceptions, the HIR is usually less than 25% and most frequently is in the 10-20% range, meaning 75-90% of kernels are diploid. With some low frequency, aneuploid embryos are also produced during haploid induction. Examples of compounds that have been shown to increase the number of haploids formed and/or the rate of haploid formation during haploid inducer crosses without increasing the rate of embryo abortion include methyl alpha linolenyl fluorophosphonate (MALFP), methyl arachidonyl fluorophosphonate (MAFP), linoleic acid (LLA), and linoleic acid ethyl ester (LLAEE). Second, one can decrease the rate of kernel abortion and fertilization failure during haploid induction, leading to the formation of more kernels per ear. Kernel abortion occurs after a successful fertilization occurs but when a functional embryo or endosperm fails to develop, and as a result a small, colorless kernel grows lacking a mature embryo inside. The kernel abortion rate is high in haploid induction processes—between 10-50% of fertilized ovules on an ear. This limits the number of haploid kernels one can recover per ear. Fertilization failure results when an ovule fails to be fertilized by a pollen grain, and is characterized by the absence of any kernel development post-pollination. This rate tends to be high during haploid induction—between 10-70% of kernels on some haploid induced ears fail to be fertilized, depending on the alignment of the male and female flower maturity, the type of cross made, and the male and female genetics. Examples of compounds that have shown to increase the rate of viable kernel formation by reducing fertilization failure and embryo abortion include the same four mentioned above: MALFP, MAFP, LLA, and LLAEE. These four molecules, when applied to pollen, tassels or other flower parts during haploid induction crosses, increase the number of haploids formed by increasing both the haploid induction rate and the kernel count.

Finally, one could improve the haploid inducer process by negating the need for haploid inducer males, by enabling de novo haploid induction to occur on an ear or in an ovule when it is self-crossed or crossed by pollen from a non-haploid inducer line. These de novo haploid induction methods, including applications of phospholipids such as DSPC, fatty acids such as linolenic acid (LNA), common mixtures of triglycerides such as corn oil and linseed oil, or phospholipase inhibitors such as MALFP, could be applied in any outcross or self-pollination to induce haploids in corn. Here we describe examples that fall into one or more of these categories, constituting improvements to or new inventions in the haploid induction process.

The present invention is directed to a method for inducing haploid embryos in a cross between two parent plants. This is done by altering the expression of a phospholipase in one of the parent plants. This altering may be accomplished in several ways: either by causing one of the parent plants to express a mutated phospholipase; or by administering a small interfering RNA to one or both of the parent plants, which causes suppression of the phospholipase; or by transforming one of the parent plants with a mutated phospholipase; or by editing one of the parent plants' phospholipase, for example by site-directed mutagenesis such as CRISPR- or TALEN-based technologies. When the phospholipase's expression in one of the parent plants is altered by one of these techniques, then when that parent plant is used in a cross, at least one haploid embryo is produced.

In one embodiment of the method, the phospholipase is a patatin-like phospholipase. In another embodiment, the patatin-like phospholipase is an orthologue of pPLAIIα, which is encoded by a nucleotide sequence comprising SEQ ID NO: 1 or a sequence at least 70% identical to SEQ ID NO: 1. The nucleotide sequence encoding the patatin-like phospholipase may be mutated, and in one embodiment the nucleotide sequence has a frameshift mutation which creates an artificial stop codon. The frameshift mutation sequence comprises SEQ ID NO: 3 or a sequence at least 70% identical to SEQ ID NO: 3.

Other mutations are possible and are within the scope of the invention. Using site-directed mutagenesis can be used to create more mutations of a phospholipase. CRISPR/Cas9, TALENs, zinc fingers, and meganucleases are methods of accomplishing site-directed mutagenesis in accordance with embodiments of the invention.

The present invention is useful in many types of crosses between plants. In one embodiment, the parent plants used in the cross are monocot plants, such as maize, rice, barley, and wheat. The parent plants may be of the same monocot species, or they may different species. In another embodiment, the parent plants used in the cross are dicot plants, such as soybean, sunflower, tomato, pepper, sugar beet, or Brussels sprouts. In a preferred embodiment, the parent plants are maize or rice plants. Within the scope of this invention are the haploid embryo produced by the method, the haploid seed comprising the haploid embryo, and the haploid plant grown from the haploid seed. Also within the scope of this invention is a doubled haploid produced by exposing the haploid embryo to a chromosome doubling agent, such a colchicine or trifluralin.

The present invention is directed to a cDNA comprising SEQ ID NO: 3, or a sequence orthologous to SEQ ID NO: 3, or a sequence 70% identical to SEQ ID NO: 3. In preferred embodiments, the sequence orthologous to SEQ ID NO: 3 encompasses patatin-like phospholipases from maize, rice, wheat, soybean, and sunflower. In particular, a sequence orthologous to SEQ ID NO: 3 includes the rice gene Os03g27610. In a more preferred embodiment, the sequence orthologous to SEQ ID NO: 3 encompasses SEQ ID NOs: 23 and 73-81.

The present invention is directed to a plant containing a human-induced, non-transgenic mutation within its patatin-like phospholipase gene. In a preferred embodiment, the patatin-like phospholipase gene is a pPLAIIα. In another embodiment, the mutation causes a premature stop codon to be encoded in the gene. In a more preferred embodiment, the plant is any monocot or any dicot, but especially preferred is maize or rice.

The present invention is also directed to a method of inducing haploid embryos and seed production by treating plant reproductive tissues with a compound comprising a lipid or a phospholipase inhibitor. In one embodiment, the treatment occurs before, during, or immediately after pollination. The plants treated may be any monocot or dicot, but in preferred embodiments the plants are maize, rice, wheat, soybean, sunflower, and sugar beet. In another embodiment, the lipid or phospholipase inhibitor is selected from the group found in Table 7. In preferred embodiments, the treatment compound comprises methyl alpha-linolenoyl fluorophosphonate (“MALFP”), linoleic acid ethyl ester (“LLAEE”), linoleic acid (“LLA”), corn oil, distearyl-phosphatidyl choline (“DSPC”), or methyl arachidonyl fluorophosphonate (“MAFP”).

In another embodiment, the lipid or phospholipase inhibitor matches the following formula (I):

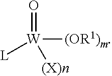

Within the scope of the invention, W of formula (I) is carbon (“C”), phosphorus (“P”), or sulfur (“S”); m may be 0 or 1; n may be 0 or 1; X is selected from the group consisting of OH, CN, O(C₁C₄ alkyl), halogen, C₁-C₄ alkyl, and C₁-C₄ alkyl substituted by one, two, or three halogen or carbonyl; R¹ is selected from the group consisting of H, C₁-C₆ alkyl, and C₁-C₆ alkyl substituted by one or more hydroxyl groups wherein optionally one or more of said hydroxyl groups is esterified with a radical independently selected from the group consisting of:

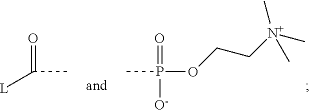

or R1 is a bond to W when W is S; each L is independently a C₂-C₃₀ carbon chain, said carbon chain optionally comprising one or more groups independently selected from alkenyl, alkynyl, phenyl, and heteroaryl, and said carbon chain optionally interrupted by 1-6 oxygen atoms. When W is C and m is 1, then n is 0; however, when W is C and m is 0, then n is 1.

In a preferred embodiment, X is F, Cl, CF₃, CCl₃, CF₂H, CCl₂H, CF₂CF₃, CCl₂CCl₃, CF₂Cl, CF₂CH₃, C(O)CH₃ or CN. In a more preferred embodiment, the halogen is F or Cl.

It is important to note that in R¹, C₁-C₆ alkyl includes linear, branched, and cyclic alkyl groups. In a preferred embodiment, R¹ is C₁-C₆ alkyl substituted by one to six hydroxyl groups.

In one embodiment, each L of formula (I) is independently a C₂-C₃₀ carbon chain, including branched chains, which may be saturated, unsaturated, or polyunsaturated. In a preferred embodiment, the carbon chain of L comprises one to four groups independently selected from alkenyl, alkynyl, phenyl, and heteroaryl. Unsaturation is in the form of double or triple bonds. The alkenyl or alkynyl can be within the carbon chain, or terminal with respect to the carbon chain. Phenyl and/or heteroaryl rings can be joined in the carbon chain at the ortho, meta, or para position, or can be terminal to the carbon chain. Aryl rings may optionally be substituted. In a preferred embodiment, the carbon chain is interrupted by one to six oxygen atoms. As used herein, “interrupted by” means that the carbon chain comprises at least two carbons in sequence, followed by an oxygen atom. For example, —CH₂—CH₂—O—CH₂—CH₂—CH₃ is a carbon chain interrupted by an oxygen atom. In a preferred embodiment, the carbon chain is interrupted by one to two oxygen atoms.

Examples of suitable carbon chains compliant with the requirements of L include: (CH₂)₈-(CH)₂-CH₂—(CH)₂-CH₂—(CH)₂-CH₂—CH₃; (CH₂)₃-(CH)₂-CH₂—(CH)₂-CH₂—(CH)₂-CH₂—(CH)₂-(CH₂)₄-CH₃; (CH₂)₇-(CH)₂-(CH₂)₇-CH₃; (CH₂)₈-(CH)₂-CH₂-phenyl-CH₂—(CH)₂-CH₂—CH₃; (CH₂)8-(CH)₂-(CH₂)₂-O—CH₂-(CH)₂—CH₂—CH₃; and (CH₂)₈-(CH)₂-CH₂-phenyl-O—(CH₂)₃-CH₃.

The treatment of these compounds is accomplished by applying the compound by any of the following techniques: dipping, injection, spray-based topical application, nebulizer, pipette-based topical application, and brush-based topical application, and any other topical application. Preferred embodiments use a spray or a nebulizer.

The present invention is further directed to a method of increasing seed set and reducing embryo abortion in plants during haploid induction, comprising treating plant reproductive tissues, such as silks, tassels, pollen, ears, kernels, or other flowering tissues, with a suitable concentration of compound prior to, during, or following pollination. In one embodiment, the compound is selected from the group consisting of the members of Table 7. In another embodiment, the compound is methyl alpha-linolenyl fluorophosphonate (MALFP). In another embodiment, the compound is linoleic acid (LLA), linoleic acid ethyl ester (LLAEE), linolenic acid (LNA), distearoyl-phosphatidylcholine (DSPC), or methyl arachidonyl fluorophosphonate (MAFP).

The present invention is further directed to a method of increasing the rate of haploid induction in a plant, comprising applying a lipid composition to tissues of the plant immediately preceding, during, or immediately following pollination. In one embodiment, the plant is a monocot or a dicot; or the plant is a maize plant or a rice plant. In another embodiment, the lipid acts as a phospholipase inhibitor and/or a fatty acid desaturase inhibitor. In another embodiment, the lipid is a fatty acid (e.g., LLA) or fatty acid ester (e.g., LLAEE) of a particular chain length and degree of saturation (eighteen carbons, and two double bonds), which is a class of fatty acid chain length that is lacking in haploid inducer pollen. By way of illustration and not limitation, the lipid is, for example, the phospholipase inhibitor methyl alpha linolenyl fluorophosphonate (MALFP), dissolved in a buffered DMSO solution at concentrations of MALFP between 0.0001 mg/mL and 1 g/mL, or dissolved in a surfactant formulation and then emulsified in a buffered dimethylactamide (DML) solution at concentrations of MALF between 0.0001 mg/mL and 1 g/mL. By way of further illustration and not limitation, the lipid composition is applied by dipping, injection, spray, mist, nebulization, pouring, brush, or any other method of application on the reproductive tissues of the plant. In one embodiment, the lipid composition is combined with pollen in a mixture, which mixture is then applied to the tissues of the plant. In another embodiment, the mixture is applied to the reproductive tissues of the plant, for example, the pollen or silks of a maize plant.

The present invention is directed to a method of inducing de novo haploid induction in a plant, comprising administering a lipid compound to at least a reproductive tissue of the plant during pollination, preceding pollination, or following pollination. In one embodiment, the plant is selected from the group consisting of monocots and dicots. In another embodiment, the plant is selected from the group consisting of rice, maize, wheat, sorghum, tomato, sugar beet, millet, barley, soybean, sunflower, cotton, oats, tobacco, vegetables, fruits, and any other crop plant.

In accordance with one exemplary embodiment, this invention includes a maize or a rice plant capable of inducing haploidy due to a human-induced mutation in the patatin-like phospholipase Allα (“PLA”) gene, as well as seeds, pollen, plant parts and progeny of that plant.

In accordance with yet another exemplary embodiment, this invention includes a maize or a rice plant capable of inducing haploids created by the steps of obtaining plant material from a parent maize or rice plant, inducing at least one mutation in at least one copy of a PLA gene of the plant material by treating the plant material with a mutagen to create mutagenized plant material, culturing the mutagenized plant material to produce progeny rice or maize plants, analyzing progeny rice or maize plants to detect at least one mutation in at least one copy of a PLA gene, selecting progeny rice or maize plants that have capability to induce haploids compared to the parent rice or maize plant; and repeating the cycle of culturing the progeny rice or maize plants to produce additional progeny plants having capability to induce haploids.

### BRIEF DESCRIPTION OF THE SEQUENCES IN THE SEQUENCE LISTING

SEQ ID NO: 1 is the cDNA sequence of an unmutated phospholipase found in GRMZM2G471240-NIL. The unmutated phospholipase allele is herein renamed MATRILINEAL.

SEQ ID NO: 2 is the amino acid sequence encoded by SEQ ID NO: 1

SEQ ID NO: 3 is the cDNA nucleotide sequence of a mutated phospholipase found in GRMZM2G471240-mtl, comprising a 4 base pair insertion. The mutated phospholipase allele is herein renamed matrilineal.

SEQ ID NO: 4 is the amino acid sequence encoded by SEQ ID NO: 3.

SEQ ID NO: 5 is the GRMZM2G471240_nil.F1 primer.

SEQ ID NO: 6 is the GRMZM2G471240_nil.R1 primer.

SEQ ID NO: 7 is the GRMZM2G471240_rwk.F1 primer.

SEQ ID NO: 8 is the GRMZM2G471240 rwk.R1 primer.

SEQ ID NO: 9 is the nucleotide sequence for the TALEN-induced MTL mutation in Event 39A ID T1 individual 22808-3954 allele 1.

SEQ ID NO: 10 is the nucleotide sequence for the TALEN-induced MTL mutation in Event 23A T1 individual ID 22808-3924 allele 1.

SEQ ID NO: 11 is the nucleotide sequence for the TALEN-induced MTL mutation in Event 81A T1 individual ID 22808-3932, Event 81A individual ID 22808-3317, and Event 81A individual ID 22808-3303.

SEQ ID NO: 12 is the nucleotide sequence for the TALEN-induced MTL mutation in Event 39A ID 22808-3954 allele 2.

SEQ ID NO: 13 is the nucleotide sequence for the TALEN-induced MTL mutation in Event 23A ID 22808-3924 allele 2.

SEQ ID NO: 14 is the nucleotide sequence for the TALEN-induced MTL mutation in Event 38A T1 individual ID 22808-4108 allele 1.

SEQ ID NO: 15 is the nucleotide sequence for the CRISPR-induced MTL mutation in Event 18A T1 individual ID 22807-4016.

SEQ ID NO: 16 is the nucleotide sequence for the CRISPR-induced MTL mutation in Event 27A T1 individual ID 22807-4073 allele 1.

SEQ ID NO: 17 is the nucleotide sequence for the CRISPR-induced MIL mutation in Event 27A T1 individual ID 22807-4081 allele 1.

SEQ ID NO: 18 is the nucleotide sequence for the CRISPR-induced MTL mutation in Event 76A T1 individual ID 22873-3999.

SEQ ID NO: 19 is the nucleotide sequence for the CRISPR-induced MTL mutation in Event 32A T1 individual ID 22873-3991.

SEQ ID NO: 20 is the nucleotide sequence for a CRISPR guide RNA.

SEQ ID NO: 21 is the genomic nucleotide sequence for Os03g27610, the rice PLA2 ortholog.

SEQ ID NO: 22 is the cDNA sequence for SEQ ID NO: 21.

SEQ ID NO: 23 is the amino acid sequence encoded by SEQ ID NO: 22.

SEQ ID NO: 24 is the nucleotide sequence of unmutated GRMZM2G471240-B73.

SEQ ID NO: 25 is the nucleotide sequence of unmutated GRMZM2G471240-RWK.

SEQ ID NO: 26 is the nucleotide sequence of unmutated GRMZM2G471240-ST6.

SEQ ID NO: 27 is the amino acid sequence encoded by SEQ ID NO: 24.

SEQ ID NO: 28 is the amino acid sequence encoded by SEQ ID NO: 25.

SEQ ID NO: 29 is the amino acid sequence encoded by SEQ ID NO: 26.

SEQ ID NO: 30 is the nucleotide sequence for the expression cassette of construct 22466, comprising wildtype MATRILINEAL.

SEQ ID NO: 31 is the nucleotide sequence for the expression cassette of construct 22467, comprising wildtype PHOSPHOGLYCERATE MUTASE.

SEQ ID NO: 32 is the nucleotide sequence for the expression cassette of construct 22503, comprising a sequence encoding a stem-loop structure targeting exon 2 of MATRILINEAL.

SEQ ID NO: 33 is the nucleotide sequence for the expression cassette of construct 22513, comprising a sequence encoding a stem-loop structure targeting exon 4 of MATRILINEAL.

SEQ ID NO: 34 is the nucleotide sequence for the expression cassette of construct 22807, comprising sequences encoding CRISPR/Cas9 editing machinery targeting MATRILINEAL in NP2222.

SEQ ID NO: 35 is the nucleotide sequence for the expression cassette of construct 22808, comprising sequences encoding CRISPR/Cas9 editing machinery targeting MATRILINEAL in NP2222.

SEQ ID NO: 36 is the nucleotide sequence for the expression cassette of construct 22873, comprising sequences encoding CRISPR/Cas9 editing machinery targeting MATRILINEAL in NP2222.

SEQ ID NO: 37 is the nucleotide sequence for the expression cassette of construct 23123, comprising sequences encoding TALEN editing machinery targeting MATRILINEAL in NP2222.

SEQ ID NO: 38 is the nucleotide sequence for the expression cassette of construct 23501, rice gRNA targeting exon 4 with dual guides.

SEQ ID NO: 39 is the nucleotide sequence for the expression cassette of construct 23501, rice gRNA targeting exon 4 single guide.

SEQ ID NO: 40 is the nucleotide sequence for the expression cassette of construct 23501, rice gRNA targeting exon 1 with dual guides.

SEQ ID NO: 41 is the nucleotide sequence for the expression cassette of construct 23501, rice gRNA targeting exon 1 with single guide.

SEQ ID NO: 42 is the nucleotide sequence for the TALEN-induced MTL mutation in Event 38A ID 22808-4108 allele 2.

SEQ ID NO: 43 is the nucleotide sequence for the CRISPR-induced MIL mutation in Event 27A ID 22807-4073 allele 2.

SEQ ID NO: 44 is the nucleotide sequence for the CRISPR-induced MTL mutation in Event 27A ID 22807-4081 allele 2.

SEQ ID NO: 45 is the nucleotide sequence for TILLING line 1139.

SEQ ID NO: 46 is the nucleotide sequence for TILLING line 3594.

SEQ ID NO: 47 is the nucleotide sequence for TILLING line 0505.

SEQ ID NO: 48 is the nucleotide sequence for TILLING line 2658.

SEQ ID NO: 49 is the nucleotide sequence for TILLING line 1983.

SEQ ID NO: 50 is the nucleotide sequence for TILLING line 2732.

SEQ ID NO: 51 is the nucleotide sequence for TILLING line 2414.

SEQ ID NO: 52 is the amino acid sequence encoded by SEQ ID NO: 45.

SEQ ID NO: 53 is the amino acid sequence encoded by SEQ ID NO: 46.

SEQ ID NO: 54 is the amino acid sequence encoded by SEQ ID NO: 47.

SEQ ID NO: 55 is the amino acid sequence encoded by SEQ ID NO: 48.

SEQ ID NO: 56 is the amino acid sequence encoded by SEQ ID NO: 49.

SEQ ID NO: 57 is the amino acid sequence encoded by SEQ ID NO: 50.

SEQ ID NO: 58 is the amino acid sequence encoded by SEQ ID NO: 51.

SEQ ID NO: 59 is the amino acid sequence encoded by SEQ ID NO: 9.

SEQ ID NO: 60 is the amino acid sequence encoded by SEQ ID NO: 10.

SEQ ID NO: 61 is the amino acid sequence encoded by SEQ ID NO: 11.

SEQ ID NO: 62 is the amino acid sequence encoded by SEQ ID NO: 12.

SEQ ID NO: 63 is the amino acid sequence encoded by SEQ ID NO: 13.

SEQ ID NO: 64 is the amino acid sequence encoded by SEQ ID NO: 14.

SEQ ID NO: 65 is the amino acid sequence encoded by SEQ ID NO: 15.

SEQ ID NO: 66 is the amino acid sequence encoded by SEQ ID NO: 16.

SEQ ID NO: 67 is the amino acid sequence encoded by SEQ ID NO: 17.

SEQ ID NO: 68 is the amino acid sequence encoded by SEQ ID NO: 18.

SEQ ID NO: 69 is the amino acid sequence encoded by SEQ ID NO: 19.

SEQ ID NO: 70 is the amino acid sequence encoded by SEQ ID NO: 42.

SEQ ID NO: 71 is the amino acid sequence encoded by SEQ ID NO: 43.

SEQ ID NO: 72 is the amino acid sequence encoded by SEQ ID NO: 44.

SEQ ID NO: 73 is the amino acid sequence for MTL ortholog found in Sorghum bicolor.

SEQ ID NO: 74 is the amino acid sequence for MTL ortholog found in Setaria italica.

SEQ ID NO: 75 is the amino acid sequence for MTL ortholog found in Hordeum vulgare.

SEQ ID NO: 76 is the amino acid sequence for MTL ortholog found in Brachypodium distachyon.

SEQ ID NO: 77 is the amino acid sequence for MTL ortholog found in Oryza sativa v. indica.

SEQ ID NO: 78 is the amino acid sequence for MTL ortholog found in Triticum aestivum.

SEQ ID NO: 79 is the amino acid sequence for MTL ortholog found in Musa acuminata.

SEQ ID NO: 80 is the amino acid sequence for MTL ortholog found in Elaeis guineensis.

SEQ ID NO: 81 is the amino acid sequence for MTL ortholog found in Arabidopsis thaliana.

### BRIEF DESCRIPTION OF THE FIGURES

FIG. 1 is a mapping scheme used to map the haploid induction trait in RWK.

FIG. 2 shows fine mapping narrowed the major QTL to a very small interval in bin 1.04, between 67.85 Mb and 68.42 Mb. This region has seven annotated genes. We sequenced and assembled the genes in this interval in several lines. The two genes with the most dramatic mutations in the haploid inducer lines are shown on the bottom right (GRMZM2G471240 and GRMZM2G062320).

FIG. 3 shows the difference in expression of GRMZM2G471240 in haploid inducer and non-inducer pollen and post-anthesis anther sacs (sporophytic tissue with the pollen grains removed). This gene is specifically expressed in the male gametophyte.

FIG. 4a shows splice-specific qRT-PCR results for GRMZM2G471240. Three biological replicates of R1-staged anthers were tested in technical triplicate, and the average Ct and standard deviation was calculated for each reaction. The relative quantity of each transcript type was compared to the endogenous control using a log₂ regression of the delta Ct. Two sets of primers were used to assess the relative abundance of each of the two annotated splice variants compared to a primer set that is agnostic with respect to the splice variants. The shorter transcript variant had relatively low abundance compared to the long transcript in both NP2222 (wild type) and NP2222-HI (haploid inducer) genotypes. Expression of the mutant copies of the gene in NP2222-HI was significantly higher for all three primer pairs tested.

FIG. 4b shows five biological replicates of fresh pollen from NP2222 and MTL^{TAL-FS} plants that are homozygous for edited mtl-like alleles) were tested in technical triplicate on the generic primer, and the average Ct and standard deviation was calculated for each reaction. The relative quantity of each transcript type was compared to the endogenous control using a log₂ regression of the delta Ct. MTL^{TAL-FS} pollen has lower transcript abundance than NP2222 (wild type) pollen.

FIG. 5a shows an amino acid alignment of the B73 predicted protein sequence of the long splice variant of the GRMZM2G471240 gene in B73 and RWK-NIL, with the predicted sequence of the mtl allele found in RWK and Stock 6 (S6). Amino acids that differ are in red; amino acids that match are indicated in normal grey text, and stop codons are indicated with a full stop. Two point mutations result in amino acid substitutions, a histidine (H) to a tyrosine (Y), and a lysine (K) to an arginine (N). These changes are not conservative; it is possible that one or both of these modifies the haploid induction phenotype—suggesting that an allelic series could be uncovered with further investigation of variants.

FIG. 5b shows wild type MTL and mutant (truncated) MTL encoded by the mtl allele have in vitro phospholipase activity. PLA2 phospholipase activity as measured by fluorescent liposome assay on recombinant, purified protein produced using the MTL and mtl cDNAs. Error bars indicate standard error based on the average of four replicates.

FIG. 6 shows mtl is responsible for pleiotropic phenotypes associated with haploid induction. 6A: Pollen tube germination rate was similar in inducers and non-inducers (n=200). 6B: Initial pollen tube elongation was also similar (n=25). 6C: RWK but not RWK-NIL is subject to segregation distortion (SD) based on low (25%) trait transmission in germinated progeny (n=300). 6D: MTL/0 complementation lines also exhibit SD against mtl in germinated progeny (n=400). 6E: Venn diagram showing RNA-seq profiling results of two haploid inducer-near isogenic pairs (left, RWK versus RWK-NIL; right, NP2222-HI versus NP2222; red text, up-regulated; green text, down-regulated). Only 60 genes were found significantly changed in the same direction.

FIG. 7 shows an amino acid alignment of the maize MTL gene to publically available MTL orthologs in eight grasses, two non-grass monocots, and Arabidopsis (thale cress). This alignment includes maize (Zea mays), sorghum (Sorghum bicolor, 92% sequence identity to MTL), foxtail millet (Setaria italica, 85% identity), barley (Hordeum vulgare, 78% identity) , Brachypodium distachyon (78% identity), Indica and Japonica variety rice (Oryza sativa v. indica and japonica, Os3g27610, 78 and 79% identity, respectively), bread wheat (Triticum aestivum, 55% identity), banana (Musa acuminata, 57% identity), oil palm (Elaeis guineesnsis, 56% identity), and Arabidopsis thaliana (52% identity).

FIG. 8. Expression profile of rice phospholipases (adapted from Singh, A., et al., Rice phospholipase A superfamily: organization, phylogenetic and expression analysis during abiotic stresses and development, PLOS ONE 7: e30947 (2012)). The closest homolog to MTL is the rice gene OspPLAIIφ (Os3g27610).

FIG. 9. Diagram showing a route to editing Os3g27610 in order to make haploid inducer lines. One could target any part of the gene (shown here—targeting the first and fourth exons) and expect to create frame-shift mutations that would lead to knockout and loss of function of the gene, and that will lead to haploid induction.

FIG. 10 shows the atomic structure of methyl alpha-linolenoyl fluorophosphonate (MALFP).

FIG. 11 shows the atomic structure of methyl arachidonyl fluorophosphonate (MAFP).

FIG. 12 shows the atomic structure of palmityl trifluoromethylketone (PACOCF3).

FIG. 13 shows the atomic structure of arachidonyl trifluoromethylketone (AACOCF3).

FIG. 14 shows the atomic structure of manoalide.

FIG. 15 shows the atomic structure of linoleic acid ethyl ester (LLAEE).

FIG. 16 shows the atomic structure of linolenic acid ethyl ester (LNAEE).

FIG. 17 shows the atomic structure of arachidonic acid methyl ester (AAME).

FIG. 18 shows the atomic structure of oleic acid methy ester (OAME).

FIG. 19 shows the atomic structure of oleic acid ethyl ester (OAEE).

FIG. 20 shows the atomic structure of palmitic acid ethyl ester (PAEE).

FIG. 21 shows the atomic structure of palmitoleic acid ethyl ester (PLAEE).

FIG. 22 shows the atomic structure of alpha-linolenic acid (aLNA).

FIG. 23 shows the atomic structure of gamma-linolenic acid (gLNA)

FIG. 24 shows the atomic structure of oleic acid.

FIG. 25 shows the atomic structure of Linoleic acid.

FIG. 26 shows the atomic structure of Arachidonic acid.

FIG. 27 shows the atomic structure of Stearic Acid.

FIG. 28 shows the atomic structure of 9(Z)-11(E)-conjugated Linoleic acid.

FIG. 29 shows the atomic structure of Distearoyl phosphatidylcholine (DSPC).

FIG. 30 shows the atomic structure of 2-oleoyl-1-palmitoyl-sn-glycero-3-phospho-ethanolamine.

FIG. 31 shows the generic atomic structure for molecules operable in the claimed invention.

### DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

As used herein, the phrase “associated with” refers to a recognizable and/or assayable relationship between two entities. For example, the phrase “associated with HI” refers to a trait, locus, gene, allele, marker, phenotype, etc., or the expression thereof, the presence or absence of which can influence an extent and/or degree at which a plant or its progeny exhibits HI. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with HI” refers to a marker whose presence or absence can be used to predict whether and/or to what extent a plant will display haploid induction.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to nucleic acids that encode polypeptides comprising amino acid sequences that are at least 95% identical to a SEQ ID NO: 2 or 3. It is understood that the disclosed subject matter thus also encompasses nucleic acids that encode polypeptides that in some embodiments consist essentially of amino acid sequences that are at least 95% identical to that SEQ ID NO: 2 or 3 as well as nucleic acids that encode polypeptides that in some embodiments consist of amino acid sequences that are at least 95% identical to that SEQ ID NO: 2 or 3. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

As used herein, the term “de novo haploid induction” refers to the triggering of haploid induction by the introduction of a spontaneous-haploid inducing agent. Such introduction can be achieved by topical spray, hand-pollination, mutagenesis, or transgenic methods. The terms “de novo haploid induction,” “de novo HI,” and “haploid induction de novo” are used interchangeably throughout this specification.

As used herein, the term “gene” refers to a hereditary unit including a sequence of DNA that occupies a specific location on a chromosome and that contains the genetic instruction for a particular characteristic or trait in an organism.

A “genetic map” is a description of genetic linkage relationships among loci on one or more chromosomes within a given species, generally depicted in a diagrammatic or tabular form.

As used herein, a plant referred to as “haploid” has a single set (genome) of chromosomes and the reduced number of chromosomes (n) in the haploid plant is equal to that of the gamete. As used herein, a plant referred to as “doubled haploid” is developed by doubling the haploid set of chromosomes. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

As used herein, the term “human-induced mutation” refers to any mutation that occurs as a result of either direct or indirect human action. This term includes, but is not limited to, mutations obtained by any method of targeted mutagenesis.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a HI-associated locus. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

One example of an algorithm that is suitable for determining percent sequence identity and sequence similarity is the BLAST algorithm, which is described in Altschul et al., 1990. In some embodiments, a percentage of sequence identity refers to sequence identity over the full length of one of the gDNA, cDNA, or the predicted protein sequences in the largest ORF of SEQ ID No: 1 being compared. In some embodiments, a calculation to determine a percentage of nucleic acid sequence identity does not include in the calculation any nucleotide positions in which either of the compared nucleic acids includes an “N” (i.e., where any nucleotide could be present at that position).

The term “open reading frame” (ORF) refers to a nucleic acid sequence that encodes a polypeptide. In some embodiments, an ORF comprises a translation initiation codon, a translation termination (i.e., stop) codon, and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

Patatin-like phospholipase A2α may also be known as PLA, pPLA, pPLAIIA pPLAIIα, PLA2alpha, or PLA2, or other similar variation. Patatin-like phospholipase AIIα is also referred to as MATRILINEAL. These terms are used interchangeably throughout. A MATRILINEAL gene comprising a four basepair frameshift mutation is hereby named matrilineal.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from a vegetative or sexual reproduction from one or more parent plants. In haploid induction the seed on the female parent is haploid, thus not a progeny of the inducing haploid line. The progeny of the haploid seed is not the only desired progeny. There is also the HI seed and subsequent plant and seed progeny of the haploid inducing plant. Both the haploid seed and the HI seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F₁ or F2 or still further generations. An F₁ is a first-generation progeny produced from parents at least one of which is used for the first time as donor of a trait, while progeny of second generation (F₂) or subsequent generations (F₃, F₄, and the like) are specimens produced from selfings, intercrosses, backcrosses, and/or other crosses of F₁s, F₂s, and the like. An F₁ can thus be (and in some embodiments is) a hybrid resulting from a cross between two true breeding parents (i.e., parents that are true-breeding are each homozygous for a trait of interest or an allele thereof), while an F₂ can be (and in some embodiments is) a progeny resulting from self-pollination of the F₁ hybrids.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer in some embodiments to a meiotic crossover.

As used herein, the term “reference sequence” refers to a defined nucleotide sequence used as a basis for nucleotide sequence comparison. In some embodiments, any of SEQ ID NOs: 1-4,22-23, or 73-81 can serve as a reference sequence for comparing to other sequences obtained from plants.

As used herein, the term “regenerate,” and grammatical variants thereof, refers to the production of a plant from tissue culture.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances.

Longer sequences typically hybridize specifically at higher temperatures. An extensive guide to the hybridization of nucleic acids is found in Sambrook & Russell, 2001. Generally, stringent conditions are selected to be about 5-10° C. lower than the thermal melting point (Tₘ) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides).

Stringent conditions can also be achieved with the addition of destabilizing agents such as formamide. Additional exemplary stringent hybridization conditions include 50% formamide, 5× SSC, and 1% SDS incubating at 42° C.; or SSC, 1% SDS, incubating at 65° C.; with one or more washes in 0.2× SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the term “trait” refers to a phenotype of interest, a gene that contributes to a phenotype of interest, as well as a nucleic acid sequence associated with a gene that contributes to a phenotype of interest. For example, a “HI trait” refers to a haploid induction phenotype as well as a gene that contributes to a haploid induction and a nucleic acid sequence (e.g., a HI-associated gene product) that is associated with the presence or absence of the haploid induction phenotype.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

As used herein, haploid induction rate (“HIR”) means the number of surviving haploid kernels over the total number of kernels after an ear is pollinated with haploid inducer pollen.

Particular problems plague that haploid induction: increased embryo abortion rates and increased fertilization failure rates (reduced seed set rates). For these reasons, there exists a need to successfully determine the cause of HI, and to use that knowledge to determine methods of stably or increasingly creating haploid plants while simultaneously reducing fertilization failure and embryo abortions.

It is specifically contemplated that one could mutagenize a promoter to potentially improve the utility of the elements for the expression of transgenes in plants. The mutagenesis of these elements can be carried out at random and the mutagenized promoter sequences screened for activity in a trial-by-error procedure. Alternatively, particular sequences which provide the promoter with desirable expression characteristics, or the promoter with expression enhancement activity, could be identified and these or similar sequences introduced into the promoter via mutation. It is further contemplated that one could mutagenize these sequences in order to enhance their expression of transgenes in a particular species. The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory sequence. In particular, site-specific mutagenesis is a technique useful in the preparation of promoter mutants, through specific mutagenesis of the underlying DNA. RNA-guided endonucleases (“RGEN,” e.g., CRISPR/Cas9) may also be used. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

Where a clone comprising a promoter has been isolated in accordance with the instant invention, one may wish to delimit the essential promoter regions within the clone. One efficient, targeted means for preparing mutagenized promoters relies upon the identification of putative regulatory elements within the promoter sequence. This can be initiated by comparison with promoter sequences known to be expressed in similar tissue specific or developmentally unique patterns. Sequences which are shared among promoters with similar expression patterns are likely candidates for the binding of transcription factors and are thus likely elements which confer expression patterns. Confirmation of these putative regulatory elements can be achieved by deletion analysis of each putative regulatory sequence followed by functional analysis of each deletion construct by assay of a reporter gene which is functionally attached to each construct. As such, once a starting promoter sequence is provided, any of a number of different deletion mutants of the starting promoter could be readily prepared.

The invention disclosed herein provides polynucleotide molecules comprising regulatory element fragments that may be used in constructing novel chimeric regulatory elements. Novel combinations comprising fragments of these polynucleotide molecules and at least one other regulatory element or fragment can be constructed and tested in plants and are considered to be within the scope of this invention. Thus the design, construction, and use of chimeric regulatory elements is one embodiment of this invention. Promoters of the present invention include homologues of cis elements known to affect gene regulation that show homology with the promoter sequences of the present invention.

Functional equivalent fragments of one of the transcription regulating nucleic acids described herein comprise at least 50, 100, 150, 200, 250, 300, 350, 400, 450, 500, 550, 600, 650, 700, 750, 800, 850, 900, 950, or 1000 base pairs of a transcription regulating nucleic acid. Equivalent fragments of transcription regulating nucleic acids, which are obtained by deleting the region encoding the 5′-untranslated region of the mRNA, would then only provide the (untranscribed) promoter region. The 5′-untranslated region can be easily determined by methods known in the art (such as 5′-RACE analysis). Accordingly, some of the transcription regulating nucleic acids, described herein, are equivalent fragments of other sequences.

As indicated above, deletion mutants of the promoter of the invention also could be randomly prepared and then assayed. Following this strategy, a series of constructs are prepared, each containing a different portion of the promoter (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

An expression cassette as described herein may comprise further regulatory elements. The term in this context is to be understood in the broad meaning comprising all sequences which may influence construction or function of the expression cassette. Regulatory elements may, for example, modify transcription and/or translation in prokaryotic or eukaryotic organisms. The expression cassette described herein may be downstream (in 3′ direction) of the nucleic acid sequence to be expressed and optionally contain additional regulatory elements, such as transcriptional or translational enhancers. Each additional regulatory element may be operably liked to the nucleic acid sequence to be expressed (or the transcription regulating nucleotide sequence). Additional regulatory elements may comprise additional promoters, minimal promoters, promoter elements, or transposon elements which may modify or enhance the expression regulating properties. The expression cassette may also contain one or more introns, one or more exons and one or more terminators.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters. Promoters which are useful for plant transgene expression include those that are inducible, viral, synthetic, constitutive (Odell Nature 313: 810-812 (1985)), temporally regulated, spatially regulated, tissue specific, and spatial temporally regulated. Using the regulatory elements described herein, numerous agronomic genes can be expressed in transformed plants. More particularly, plants can be genetically engineered to express various phenotypes of agronomic interest.

The compounds of the present invention may exist in different geometric or optical isomers (diastereoisomers and enantiomers) or tautomeric forms. This invention covers all such isomers and tautomers and mixtures thereof in all proportions as well as isotopic forms such as deuterated compounds. The invention also covers all salts, N-oxides, and metalloidic complexes of the compounds of the present invention.

Each alkyl moiety either alone or as part of a larger group (such as alkoxy, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. The alkyl groups include C1-C6 alkyl, C1-C4 alkyl, and C1-C3 alkyl.

The term “alkenyl,” as used herein, is an alkyl moiety having at least one carbon-carbon double bond, for example C2-C6 alkenyl. Specific examples include vinyl and allyl. The alkenyl moiety may be part of a larger group (such as alkenoxy, alkenoxycarbonyl, alkenylcarbonyl, alkyenlaminocarbonyl, dialkenylaminocarbonyl).

The term “acetoxy” refers to —OC(═O)CH3.

The term “alkynyl,” as used herein, is an alkyl moiety having at least one carbon-carbon triple bond, for example C2-C6 alkynyl. Specific examples include ethynyl and propargyl. The alkynyl moiety may be part of a larger group (such as alkynoxy, alkynoxycarbonyl, alkynylcarbonyl, alkynylaminocarbonyl, dialkynylaminocarbonyl).

Halogen is fluorine (F), chlorine (Cl), bromine (Br) or iodine (I).

Haloalkyl groups (either alone or as part of a larger group, such as haloalkoxy or haloalkylthio) are alkyl groups which are substituted with one or more of the same or different halogen atoms and are, for example, —CF3, —CF2Cl, —CH2CF3, or —CH2CHF2.

Hydroxyalkyl groups are alkyl groups which are substituted with one or more hydroxyl group and are, for example, —CH2OH, —CH2CH2OH or —CH(OH)CH3.

Alkoxyalkyl groups are an alkoxy group bonded to an alkyl (R—O—R′), for example —(CH2)ᵣO(CH2)ₛCH3, wherein r is 1 to 6 and s is 1 to 5.

In the context of the present specification, the term “aryl” refers to a ring system which may be mono, bi or tricyclic. Examples of such rings include phenyl, naphthalenyl, anthracenyl, indenyl or phenanthrenyl.

Unless otherwise indicated, alkenyl and alkynyl, on their own or as part of another substituent, may be straight or branched chain and may contain 2 to 6 carbon atoms, and where appropriate, may be in either the (E) or (Z) configuration. Examples include vinyl, allyl, ethynyl and propargyl.

Unless otherwise indicated, cycloalkyl may be mono- or bi-cyclic, may be optionally substituted by one or more C1-C6 alkyl groups, and contain 3 to 7 carbon atoms. Examples of cycloalkyl include cyclopropyl, 1-methylcyclopropyl, 2-methylcyclopropyl, cyclobutyl, cyclopentyl, and cyclohexyl.

The term “heterocyclyl” refers to a ring system containing from one to four heteroatoms selected from N, O and S, wherein the nitrogen and sulphur atoms are optionally oxidized, and the nitrogen atom(s) are optionally quaternized. Heterocyclyl includes heteroaryl, saturated analogues, and in addition their unsaturated or partially unsaturated analogues such as 4,5,6,7-tetrahydro-benzothiophenyl, 9H-fluorenyl, 3,4-dihydro-2H-benzo-1,4-dioxepinyl, 2,3-dihydro-benzo-furanyl, piperidinyl, 1,3-dioxolanyl, 1,3-dioxanyl, 4,5-dihydro-isoxazolyl, tetrahydrofuranyl and morpholinyl. In addition, the term “heterocyclyl” includes heterocycloalkyl, a non-aromatic monocyclic or polycyclic ring comprising carbon and hydrogen atoms and at least one heteroatom selected from nitrogen, oxygen, and sulfur such asoxetanyl or thietanyl. A monocyclic heterocycloalkyl may contain 3 to 7 members.

The term “heteroaryl” refers to an aromatic ring system containing from one to four heteroatoms selected from N, O and S, wherein the nitrogen and sulfur atoms are optionally oxidized, for example having 5, 6, 9, or 10 members, and consisting either of a single ring or of two or more fused rings. Single rings may contain up to three heteroatoms, and bicyclic systems up to four heteroatoms, which will preferably be chosen from nitrogen, oxygen, and sulfur. Examples of such groups include pyridyl, pyridazinyl, pyrimidinyl, pyrazinyl, furanyl, thienyl, oxazolyl, isoxazolyl, oxadiazolyl, thiazolyl, isothiazolyl, thiadiazolyl, pyrrolyl, pyrazolyl, imidazolyl, triazolyl, and tetrazolyl.

### EXAMPLES

I. Identifying the Frameshift Mutation in PLA

The present invention identifies a series of independent human-induced mutations found in at least one patatin-like phospholipase AIIα (pPLAIIα) gene of maize; maize plants having these mutations in at least one of their PLA genes; and a method of creating and identifying similar and/or additional mutations in the PLA gene by screening pooled and/or individual rice and maize plants. The rice and maize plants of the present invention induce haploidy as a result of non-transgenic mutations in at least one of their PLA genes.

More specifically, the present invention produces new maize haploid-inducing lines. A number of known haploid-inducing maize lines exist including but not limited to: Stock 6, MHI (Moldovian Haploid Inducer), indeterminate gametophyte (“ig”) mutation, KEMS, ZEM, ZMS, KMS, RWS, and RWK. The present invention relates to a method of identifying, and/or selecting germplasm which can or cannot induce haploids. The present invention also relates to increasing and further development of the selected haploid inducing germplasm. The invention further relates to a method of improving haploid inducing germplasm to increase the induction of haploids on the seed producing parent.

The initial step in the production of haploid seeds from a hybrid or segregating maternal parent plant derives from the pollination with pollen from a haploid inducer onto the ear from a seed producing plant. A result of this hybridization process is the production of diploid and maternal haploid (1n) kernels. The induced haploid (1n) kernels are often distinguished from the diploid seed by the use of color markers which indicate embryo ploidy. The diploid seeds are generally discarded, while haploid kernels or embryos are often subjected to chromosome doubling processes to produce doubled haploid plants. More specifically, the haploid genetic material is treated with one or more mitotic arrest agents to allow the haploid (1n) chromosome complement in one or more cells to produce homolog-pairs. After the chemical treatment procedure, the chromosome doubling chemical(s) are removed. The now-doubled haploid maize is allowed to mature and the resulting doubled haploid seeds when planted will produce homozygous plants (also called inbred plant or lines). These inbred lines are the materials that breeders utilize to pursue their hybrid development programs.

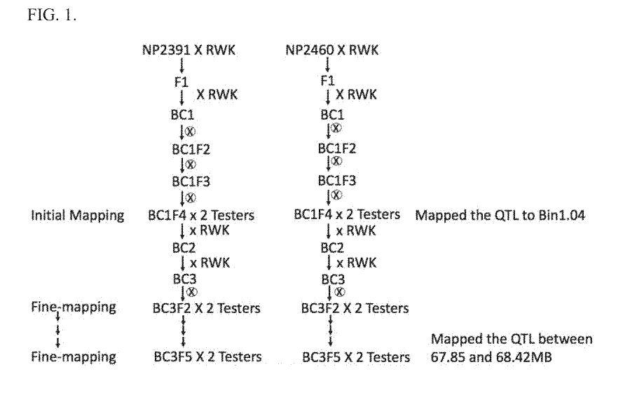

The locus for the haploid induction trait was fine mapped. Although a major QTL on chromosome 1 responsible for haploid induction has been mapped and published, Dong et al. Theor. Appl. Genet (2013) 126: 1713-1720, the exact gene/genetic element responsible for the induction process has not been identified until now. To clarify the developmental genetics underlying haploid induction, the Stock 6 derivative RWK (˜13% HIR) was obtained from the University of Hohenheim in 2006, crossed to inbreds NP2460 and NP2391, and subsequently backcrossed to RWK to generate mapping populations. See FIG. 1.

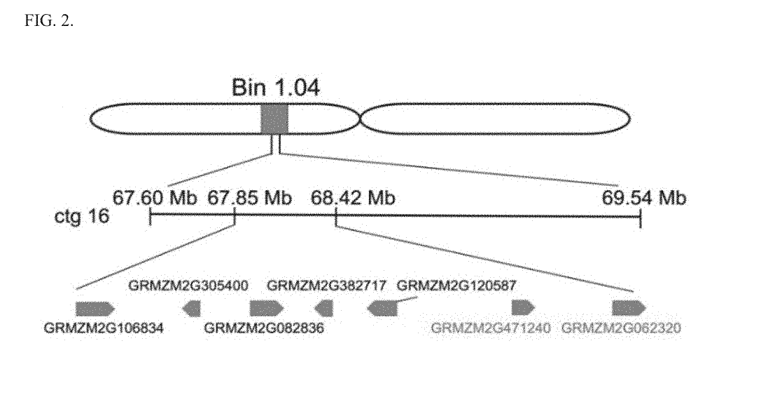

Elevated HIR in both populations co-segregated with marker SM020SDQ in bin 1.04, consistent with recent reports on a QTL called qhir1. See Prigge, et al., New Insights into the Genetics of in Vivo Induction of Maternal Haploids, the Backbone of Doubled Haploid Technology in Maize, GENETICS (2012) 190:781-793 (discussing major QTL for HI in Bin 1.04 [qhir1] and minor QTLs for HI in Bins 3.02 [qhir2], 3.06 [qhir3], 4.03 [qhir4], 5.01 [qhir5], 5.04 [qhir6], 7.01 [qhir7], and 9.01 [qhir8]); Liu, et al., Fine mapping of qhir8 affecting in vivo haploid induction in maize, THEOR. APPL. GENET. (2015) 128:2507-2515 (fine mapping thirty-five genes to qhir8); Hu, et al., The Genetic Basis of Haploid Induction in Maize Identified with a Novel Genome-Wide Association Method, GENETICS (2016) 202:1267-1276 (asserting that qhir1 is two QTLs: qhir11 and qhir12, and fine mapping qhir6 to a 1.1 Mb region). We did several rounds of fine mapping and narrowed the QTL to an approximately 0.57 Mb region between 67.85 Mb and 68.42 Mb that lies within qhirl 11. This region has seven annotated genes (FIG. 2).

Using the Illumina HiSeq2000, we sequenced RWK, Stock 6, and a BC3F5 non-inducer “RWK-NIL” that is near-isogenic to RWK but has NP2391 haplotypes in the qhir11 interval. By comparing inducer and non-inducer germplasm, it was determined that a four nucleotide insertion present in haploid inducers which shifts the frame for amino acid coding of GRMZM2G471240 is not present in non-inducer germplasm. Therefore, the present invention has identified a gene with a frameshift mutation in inducer germplasm as being responsible for maize haploid induction. The candidate gene corresponding to gene model GRMZM2G471240 encodes patatin-like phospholipase AIIα (pPLAIIα), which we have renamed MATRILINEAL (MTL) to represent the wildtype allele and the frameshift allele is referred to as matrilineal (mtl).

DNA sequence was generated for each candidate gene from the two inducer lines (Stock 6 and RWK) and one non-inducer line (RWK-NIL). In addition, the public B73 genome data was used as a second non-inducer line. Gene model information was compared to EST/cDNA data to confirm the structure of each gene. The annotated sequence data were compared to catalog differences between the four alleles of each gene.

The sequence comparisons revealed that B73 and RWK-NIL alleles were similar to each other, and RWK and Stock 6 alleles were similar to each other. Most sequence differences were single nucleotide polymorphisms that do not alter protein coding sequence. There were some insertions and some deletions, most of which are in non-protein coding sequence.

Table 1 below lists gene identities in the interval shown. This information is from chromosome 1, see, e.g., Patrick S. Schnable et al., The B73 Maize Genome: Complexity, Diversity, and Dynamics, 326 SCIENCE 1112-15 (2009), incorporated herein by reference in its entirety, and lists a short description of the other encoded proteins from the genes within the haploid inducing locus.

TABLE 1
Information on Chromosome 1 from Maize.

| | Transcript | | Query | Subject | | | | Align | |
|---|---|---|---|---|---|---|---|---|---|
| Gene_id | Start | End | length | length | Score | Identity | Similarity | length | Description |
| GRMZM2G305400 | 67991172 | 67994092 | 308 | 362 | 383 | 33.3 | 53.33752 | 314 | Cyclin D2; 1 |
| GRMZM2G082836 | 68107606 | 68110989 | 202 | 205 | 729 | 71.2 | 83.33333 | 198 | GTP-binding protein 1 |
| GRMZM2G382717 | 68113453 | 68115168 | 396 | 464 | 489 | 38.77 | 53.17371 | 314 | Chaperone DnaJ-domain superfamily |
| | | | | | | | | | protein |
| GRMZM2G120587 | 68133178 | 68136953 | 458 | 461 | 1329 | 55 | 71.23894 | 452 | serine carboxypeptidase-like 51 |
| GRMZM2G471240 | 68240862 | 68242656 | 428 | 407 | 1049 | 51.5 | 72.36181 | 398 | phospholipase A 2A |
| GRMZM2G471240 | 68240862 | 68242656 | 401 | 407 | 961 | 50.15 | 70.0938 | 395 | phospholipase A 2A |
| GRMZM2G062320 | 68318898 | 68321409 | 335 | 334 | 1064 | 73.3 | 84.21053 | 285 | Phosphoglycerate mutase family |
| | | | | | | | | | protein |
| GRMZM5G866758 | 68430654 | 68436197 | 401 | 403 | 1678 | 80.4 | 90.45226 | 398 | acetoacetyl-CoA thiolase 2 |
| GRMZM5G866758 | 68430634 | 68436197 | 303 | 403 | 1248 | 78.4 | 89.40397 | 302 | acetoacetyl-CoA thiolase 2 |
| GRMZM2G003530 | 68435670 | 68439997 | 360 | 344 | 1063 | 60.5 | 76.41791 | 335 | P-loop containing nucleoside |
| | | | | | | | | | triphosphate hydrolases |
| | | | | | | | | | superfamily protein |
| GRMZM2G077991 | 68343246 | 68546264 | 94 | 95 | 424 | 79.7 | 91.48936 | 94 | Zinc-binding ribosomal protein |
| | | | | | | | | | family protein |
| GRMZM2G077991 | 68543694 | 68546264 | 94 | 95 | 424 | 79.7 | 91.48936 | 94 | Zinc-binding ribosomal protein |
| | | | | | | | | | family protein |
| GRMZM2G077991 | 68543805 | 68546269 | 147 | 95 | 419 | 79.5 | 91.39785 | 93 | Zinc-binding ribosomal protein |
| | | | | | | | | | family protein |
| GRMZM2G077960 | 68354980 | 68559182 | 438 | 428 | 1422 | 65.3 | 79.80998 | 421 | Protein phosphatase 2C family |
| | | | | | | | | | protein |
| GRMZM2G077897 | 68561209 | 68565155 | 784 | 807 | 1561 | 48.1 | 65.69848 | 723 | Plant protein of unknown function |
| | | | | | | | | | (DUF827) |
| GRRZM2G347583 | 68660278 | 68665995 | 1651 | 2156 | 1201 | 41.37 | 55.70954 | 1375 | |
| GRMZM2G173030 | 68668900 | 68671460 | 626 | 2156 | 858 | 35.6 | 48.30299 | 586 | |
| GRMZM2G022061 | 68876150 | 68882226 | 203 | 556 | 618 | 64.9 | 79.89691 | 194 | |
| GRMZM2G022061 | 68876130 | 68882226 | 322 | 536 | 1004 | 66 | 77.47748 | 333 | |
| GRMZM2G022061 | 68876150 | 68882226 | 142 | 356 | 347 | 79.6 | 89.84375 | 128 | |
| GRMZM2G022061 | 68876150 | 68882226 | 322 | 556 | 1004 | 66 | 77.47748 | 333 | |
| GRMZM2G022061 | 68876150 | 68882226 | 534 | 556 | 1802 | 67.7 | 79.81651 | 545 | |
| GRMZM2G340286 | 68928213 | 68929600 | 378 | 403 | 370 | 37.83 | 55.75713 | 407 | |
| GRMZM2G340279 | 68934652 | 68937080 | 746 | 937 | 3095 | 29 .34 | 50.31745 | 2517 | Tetratricopeptide repeat (TPR)- |
| | | | | | | | | | like superfamily protein |
| GRMZM2G347808 | 69003208 | 69012612 | 589 | 435 | 1115 | 50.4 | 66.60178 | 423 | S-adenosyl-L-methionine-dependent |
| | | | | | | | | | methyltransferases superfamily |
| | | | | | | | | | protein |

Having completed fine mapping of the haploid inducer trait to an interval containing only seven genes, we focused on those in the sequence assembly and analysis. The sequences for the seven genes were nearly identical between B73 and RWK-NIL, but RWK and Stock 6 lacked GRMZM2G062320, a PHOSPHOGLYCERATE MUTASE (PGM), and had a 4 basepair (“bp”) insertion in the fourth exon of GRMZM2G471240, a PATATIN-LIKE PHOSPHOLIPASE AIIα (pPLAIIα) (FIG. 2). We found that RWK and Stock 6 both have the same 4 bp insertion in the fourth exon of pPLAIIα, and that this gene is specifically expressed in pollen (see maizegdb.org/gene_center/gene?id=GRMZM2G471240 incorporated herein by reference). The unmutated GRMZM2G471240 is represented by SEQ ID NO: 1. GRMZM2G471240, comprising the 4 bp insertion in the fourth exon, is represented by SEQ ID NO: 3.

Most of the haploids that were identified were found using a taqman marker test. This marker test takes advantage of a difference in the pPLAIIα gene between RWK×NP2222. In crosses where we use RWK as the female, and NP2222 as the male, the RWK parent is homozygous for the mtl allele, while NP2222 is homozygous for the MTL allele. Diploid progeny are MTL/mtl and haploid gynogenetic haploid progeny are mtl/0. Therefore when this test is done the taqman results show 1 copy of the mtl allele and one copy of MTL allele in the diploid progeny, and 1 copy of the mtl but no copies of MTL in the haploid progeny. When this type of cross is performed, ears are harvested between 12-21 days following pollination, the embryos are extracted and a small sample of the embryos are taken for taqman marker analysis. Alternatively the embryos are plated on solid media and germinated in the dark so that a larger sample of the extended shoot or root can be taken between 2-10 days later for marker analysis. At the same time some of the tissue is saved for ploidy analysis. In this latter case after the molecular test is used, the larger samples of the haploids can be run on a CyFlow Space ploidy analyzer and confirmed as haploids. In most cases this results in the positive identification of haploids. In a few rarer cases this results in the overturning of the false positive marker results and correction of the call as a diploid.

Another way we test for haploids is via dominant marker assay. In this case, an X26 male line is used. This line is homozygous for a marker that acts in a dominant fashion. In such a cross any line can be used as a female as long as it doesn't have a marker or any genes or alleles that work to inhibit the marker phenotype. The X26 line is a non-inducer and is homozygous for MTL. Using such a line, the progeny are dissected between 12-21 days after pollination and evaluated for the presence of the marker, or they are examined directly on the ear, or the dried kernels are harvested and evaluated for the presence of the marker. Diploid progeny show the marker phenotype because they have a single copy of the marker gene from the X26 male parent, whereas gynogenic haploid progeny do not show the marker phenotype. The penetrance of the marker and the spontaneous haploid induction rate of X26 was tested in numerous control crosses. Using this system we screen for haploids and then test them on the ploidy analyzer to confirm that they are truly haploids.

We developed PCR tests to specifically detect the “wild-type” and “mutant” alleles for screening of nineteen Stock 6-derived inducers, including NP2222-Haploid Inducer (NP2222-HI), a BC3 introgression of RWK into Syngenta's standard transformable inbred line NP2222. We also screened nine non-inducer control lines.

To develop a PCR test that would distinguish between RWK/Stock6 and RWK-NIL haplotypes, two primer pairs were designed: one pair should amplify the RWK/Stock6 frame-shift allele, while the other should amplify the B73/RWK-NIL allele. These pairs worked as expected on RWK-NIL, RWK, and Stock6 DNA: RWK-NIL gDNA only amplified the RWK-NIL primer pair. RWK and Stock6 gDNA only amplified the RWK/Stock6 primer pair, which specifically detects the frame-shift allele. The PCR products were sequenced and the sequences were identical to that from whole genome sequencing. SNPs that were identified in the whole genome sequencing were confirmed in the PCR products. Below, in FIG. 2, the DNA used in each reaction is in capital letters. The primers are “nil.F1/R1” and “rwk.F1/R1.”

GRMZM2G471240_nil.F1:
(SEQ ID NO: 5)
GTACGCCGTGCGCTAACA.
GRMZM2G471240_nil.R1:
(SEQ ID NO: 6)
TCGTACCTCCCTGTCTCCAC.
GRMZM2G471240_rwk.F1:
(SEQ ID NO: 7)
TACGCCGTGCGCTAACATA.
GRMZM2G471240_rwk.R1:
(SEQ ID NO: 8)
GTACCTCGCTCCCTGTCTCC.

The “rwk.F1/R1” and “nil.F1/R1” primer pairs were used to genotype the panel of high, low, and non-inducers. We found that all 19 haploid inducer lines had the 4 bp insertion, including Stock6 (3% haploid induction rate [“HIR”]), RWK (line derived from the University of Honheim stocks, 10-15% HIR), RWS, and Z22, among others. In contrast, the wild-type allele was found in all nine non-haploid inducer lines (average HIR of 0.1%). The data indicates that homozygosity for the frame-shift allele correlates with induction capacity: 12/12 high and 7/7 low inducers amplified the frame-shift assay, but not the wild type assay, while 9/9 non-inducers amplified the wild type but not frame-shift assay. This indicates that induction capacity correlates with the GRMZM2G471240 mutation, and that pPLAIIα underlies qhir 11 and is the primary mutation responsible for haploid induction in these lines.

TABLE 2
GRMZM2G471240 PCR test results.

| | Induc- | RWK | RWK-NIL |
|---|---|---|---|
| | tion | ampli- | ampli- |
| | Rate | con | con |
| Controls: | | | |
| Stock 6 (low inducer) | 2.50% | + | − |
| RWK (high inducer) | 12% | + | − |
| RWK-NIL (non-inducer) | <1% | − | + |
| Good Inducers: | | | |
| ZMS | 7% | + | − |
| Z19-PR | 7% | + | − |
| RWS-Z86 | 10% | + | − |
| K13 | 9% | + | − |
| (ID3002/Z22)B > 29-5 > 2-5-1-B- | 7% | + | − |
| Z-19-//AF4031PR//Z-19-)1- | 9.50% | + | − |
| 1-2-3-1-3-B- | | | |
| ZR86 | 12% | + | − |
| ZR53 | 12% | + | − |
| ZR75 | 13% | + | − |
| (Z21/RWS)B(GS)-75-1-2-3-B- | ~8% | + | − |
| NP2222 inducer-good | ~9% | + | − |
| Poor Inducers: | | | |
| Stock6 R1-nj | 2.50% | + | − |
| (Z21/RWS//[RWS]B$)33-5- | <2% | + | − |
| (K-13-/(ZMS/SEW-PR)B > 2 > | <2% | + | − |
| B-9//K-13-)2-4-1- | | | |
| (K-13-/(ZMS/SEW-PR)B > 2 > | <2% | + | − |
| B-9//K-13-)6-1-2- | | | |
| (ZMS/SEW-PR)B > 2 > B-7-2-1-2- | <2% | + | − |
| NP2222 inducer-low | ~3% | + | − |
| Non-inducer Lines and Donors: | | | |
| Stock6 Rl-nj B1Pl1 | <0.1% | − | + |
| (Z-21-/AF4031PR//Z-21-1-B-)1-1-1-1-B- | <0.1% | − | + |
| FF6096 | <0.1% | − | + |
| ID5829 | <0.1% | − | + |
| XO5744 | <0.1% | − | + |
| ID3002 | <0.1% | − | + |
| AF4031PR | <0.1% | − | + |
| NP2222 | <0.1% | − | + |

We also identified a number of single nucleotide polymorphisms (“SNPs”) between the frame-shift allele and that of RWK-NIL. For many of these SNPs, the STOCK6 and RWK sequences agreed with other inbreds we have sequenced, and thus likely represent natural variation. Indeed most of these SNPs did not alter the amino acid sequence and thus likely do not contribute to the haploid induction phenotype. Two SNPs did result in amino acid changes (H107Y; K232N) and these are not highly conservative changes, so they may have a small contribution to the phenotype, but mostly like they do not impact the phenotype because the frame-shift causes a loss of function.

We renamed pPLAIIα “MATRILINEAL” (MTL; i.e., SEQ ID NO: 1) and the native 4 bp insertion allele “matrilineal” (mtl; i.e., SEQ ID NO: 3). According to the predicted protein sequence, the 4 bp insertion causes a shift in the open reading frame of the protein at amino acid (“AA”) 352 out of 401. The frame-shift leads to a premature stop codon.

After finding the frame-shift knock-out mutation we directly tested the effect it had on haploid induction by complementing a haploid inducer line with a wild-type pPLAIIα transgene. Heterologous complementation of NP2222-HI (10.2% HIR) with a wild-type copy of MATRILINEAL virtually eliminated haploid induction and kernel abortion. Compared to controls the HIR decreased 50-fold, from 10% to 0.23%. It also decreases the embryo abortion rate to 0.65%. Full length functional reporter lines were also made using transgenic fusions of the wild type MTL gene to GFP as well as the mutant allele mtl to GFP, in order to both visualize subcellular localization of wild-type MTL, but also to see if the mutant version of the protein localizes correctly or is produced at all. These lines also served as additional material to test for complementation. Haploid inducer material (NP2222-HI) that was homozygous for the MTL-GFP transgene also did not exhibit the haploid inducer phenotype. The induction rate of NP2222-HI falls to 0.60% when it is homozygous for MTL-GFP. Additionally, the MTL-GFP transgene also knocked down embryo abortion to 4.86%. Finally we tested whether the mutant mtl allele fused to GFP complements the haploid induction phenotype, and it does not. Haploid induction and embryo abortion rates were very similar in NP2222-HI compared to NP2222-HI that was homozygous for the mutant fusion transgene mtl-GFP. See Table 3. This represents conclusive evidence that the MATRILINEAL frame-shift is responsible and required for haploid induction. To apply this knowledge, we demonstrate that mutating or modulating the expression of pPLAIIα in a wild type line leads to the creation of new haploid induction lines.

TABLE 3
Reproductive characteristics of haploid inducer, complementation and edited lines.
This table shows the haploid induction and kernel abortion rates of inducer lines in the
NP2222 background. The number testcrosses is listed first (“ears”) and then the kernel and
embryo statistics are listed. Embryo abortion and haploid induction generally comes together
on the same ear. That is why the embryo abortion rate is so high in an ear crossed by the
NP2222-HI male, which has a 10.17% haploid induction rate (HIR). Both the WT
transgenes, including one without GFP and one fused to GFP, complemented the haploid
induction phenotype. Meanwhile, the mutant mtl fused to GFP did not complement.

| Complementation Assays | | | | Kernel characteristics | | | Embryos tested | |
|---|---|---|---|---|---|---|---|---|
| Male parent | ears | viable | aborted | % aborted | embryos | haploids | diploids | HIR |
| NP2222-HI | 4 | 548 | 498 | 47.61% | 531 | 54 | 477 | 10.17% |
| NP2222-HI + MTL/MTL | 17 | 4403 | 29 | 0.65% | 4321 | 11 | 4310 | 0.25% |
| NP2222-HI + mtl-GFP/mtl-GFP | 3 | 371 | 298 | 44.54% | 360 | 34 | 326 | 9.44% |
| NP2222-HI + MTL-GFP/MTL-GFP | 3 | 1019 | 52 | 4.86% | 836 | 5 | 831 | 0.60% |

Several mtl-like alleles were generated in the inbred NP2222 by introducing small deletions in MTL close to the 4 bp insertion site in mtl, using transcription activator-like effector nucleases (TALEN) (Boch, J. et al., Breaking the code of DNA binding specificity of TAL-type III effectors, SCIENCE 326:1509-1512 (2009), incorporated herein by reference). Several mutant events were self-pollinated and T1 plants lacking the TALEN T-DNA insert but homozygous for small deletions in MTL were outcrossed onto NP2222. Edited lines homozygous for frame-shift deletions in MTL (hereafter called MTL^{TAL-FS}) exhibited an HIR of 4.0-12.5% (average 6.65%) (Table 4). The ploidy status of 118/127 putative haploids was confirmed by Flow Cytometry, and phenotypic evaluations indicated these plants were haploids. These results prove that a frame-shift in MTL is sufficient to induce high rates of haploid induction. Other contributors to the phenotype have been mapped including the neighboring qhir12 (see Liu, 2016)), which may account for the difference between the HIR of MTL^{TAL-FS} and NP2222-HI. It reasonable to infer that seed set, HIR and kernel abortion rates are set through mtl by paternal and maternal genotype-specific interactions.

TABLE 4
Reproductive characteristics of MTL edited lines (generally referred to as MTL^{TAL-FS} lines). This table shows the haploid
induction and kernel abortion rates of inducer lines in the NP2222 background. The transgenic events tested are given on the left
followed by the number of testcrosses made (“ears”) and the progeny statistics.

| | | | | Kernel Characteristics | | | | Ploidy Analysis Data | | |
|---|---|---|---|---|---|---|---|---|---|---|
| | | | | Avg. | Avg. | % | Total | Putative | Confirmed | |
| Event | ID | Mutation(s) | Ears | viable | aborted | aborted | Embryos | Haploids | Haploids | HIR |
| 39A | 3954 | Biallelic (13 bp | 4 | 162 | 128 | 44.10% | 579 | 37 | 35 | 6.04% |
| | | & 28 bp dels) | | | | | | | | |
| 23A | 3924 | Biallelic (8 bp | 2 | 114 | 116 | 50.40% | 128 | 18 | 16 | 12.50% |
| | | & 5 bp dels) | | | | | | | | |
| 81A | 3932 | Homozygous (13 | 2 | 165 | 129 | 43.90% | 169 | 18 | 15 | 8.88% |
| | | bp del) | | | | | | | | |
| 81A | 3317 | Homozygous (13 | 2 | 183 | 108 | 37.10% | 343 | 19 | 19 | 5.54% |
| | | bp del) | | | | | | | | |
| 81A | 3303 | Homozygous (13 | 1 | 189 | 100 | 34.60% | 176 | 7 | 7 | 3.98% |
| | | bp del) | | | | | | | | |
| 38A | 4108 | Biallelic (11 bp | 4 | 147 | 102 | 40.10% | 379 | 28 | 26 | 6.86% |
| | | & 5 bp dels) | | | | | | | | |
| 18A | 22807- | Homozygous (8 bp | 8 | 144 | 97 | 40.20% | 1025 | 47 | 44 | 4.29% |
| | 4016 | del) | | | | | | | | |
| 27A | 22807- | Biallelic (1 bp | 2 | 161 | 92 | 36.40% | 180 | 18 | 18 | 10.00% |
| | 4073 | insert & 5 bp | | | | | | | | |
| | | del) | | | | | | | | |
| 27A | 22807- | Biallelic (1 bp | 6 | 176 | 116 | 39.80% | 931 | 45 | 44 | 4.73% |
| | 4081 | insert & 8 bp | | | | | | | | |
| | | del) | | | | | | | | |
| 76A | 22873- | Homozygous (2 | 2 | 175 | 95 | 35.20% | 117 | 17 | 16 | 13.68% |
| | 3999 | bp | | | | | | | | |
| | | insert) | | | | | | | | |
| 32A | 22873- | Homozygous (1 | 2 | 140 | 105 | 42.90% | 260 | 14 | 14 | 5.38% |
| | 3991 | bp del) | | | | | | | | |
| Total | | Totals | 15 | 160 | 108 | 40% | 390 | 24 | 23 | 7% |

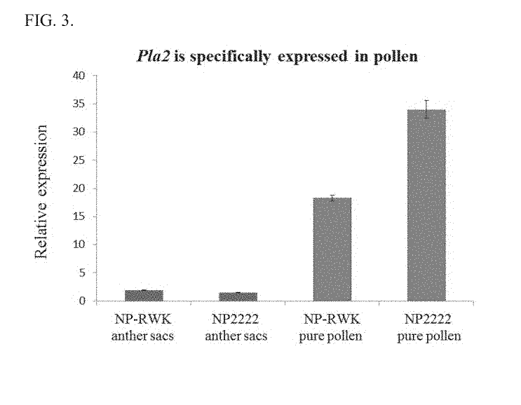

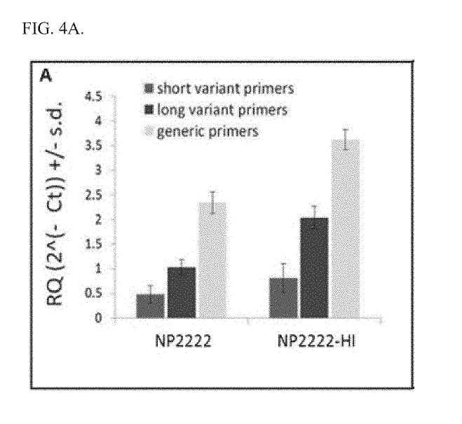

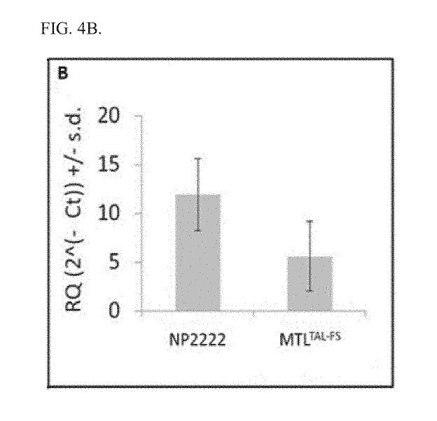

Haploid seed formation in maize is a post-zygotic character triggered by a defective male gametophyte. This fact is reflected in MTL expression data. Public RNA-seq profiles indicate the wild-type MTL transcript is specific to anthesis-staged anthers (see Sekhon, R. S., et al. Genome-wide atlas of transcription during maize development, PLANT JOURNAL, 66, 553-563 (2011), incorporated herein by reference), in agreement with a developmental profile that found it exclusively in pre-dehiscent anthers (see Zhai, J., et al. Spatiotemporally dynamic, cell-type-dependent premeiotic and meiotic phasiRNAs in maize anthers, PNAS 112, 3146-3151 (2015), incorporated herein by reference). We found that wild-type pollen had 18× more MTL transcript than post-anthesis anther sacs, indicating the gene is male gametophyte-specific (FIG. 3). Attempts to knockdown MTL by RNAi led to elevated rates of haploid formation for MTL^{RNAi} only (Table 5). There are two annotated splice variants of MTL, reflecting an alternative splice site 81 nucleotides prior to the 3′ end of exon 2. Compared to NP2222, Mtl was elevated in NP2222-HI but not MTL^{TAL-FS} pollen (FIG. 4), while the abundance of the two annotated splice variants was consistent.

TABLE 5
RNAi construct 22503 (SEQ ID NO: 32) to knockdown Mtl led to haploid induction.

| GRMZM2G471240 RNAi | Event | | Kernel Characteristics | | | Embryos tested for ploidy | | | |
|---|---|---|---|---|---|---|---|---|---|
| Individual ID | ID | ears | viable | aborted | % aborted | embryos | haploids | diploids | HIR |
| 5148 | 001 | 2 | 701 | 43 | 5.78% | 369 | 3 | 366 | 0.81% |
| 5149 | 001 | 2 | 186 | 22 | 10.58% | 166 | 1 | 165 | 0.60% |
| 5153 | 001 | 2 | 625 | 61 | 8.89% | 323 | 7 | 316 | 2.17% |
| 5161 | 001 | 3 | 1116 | 87 | 7.23% | 485 | 4 | 481 | 0.82% |
| 5170 | 028 | 2 | 629 | 23 | 3.53% | 324 | 1 | 323 | 0.31% |
| 5173 | 028 | 2 | 551 | 33 | 5.65% | 322 | 0 | 322 | 0.00% |
| 5187 | 028 | 3 | 379 | 27 | 6.65% | 333 | 9 | 324 | 2.70% |
| 3731 | 014 | 2 | 894 | 23 | 2.51% | 263 | 4 | 259 | 1.52% |
| 3732 | 014 | 2 | 648 | 49 | 7.03% | 351 | 0 | 351 | 0.00% |
| 3736 | 007 | 1 | 277 | 21 | 7.05% | 277 | 0 | 277 | 0.00% |
| 3737 | 007 | 1 | 223 | 49 | 18.01% | 175 | 3 | 172 | 1.71% |
| 3751 | 005 | 1 | 133 | 6 | 4.32% | 118 | 0 | 118 | 0.00% |
| | 5 | | | | | | | | |
| TOTALS | events | 23 | 6362 | 444 | 6.52% | 3506 | 32 | 3474 | 0.91% |

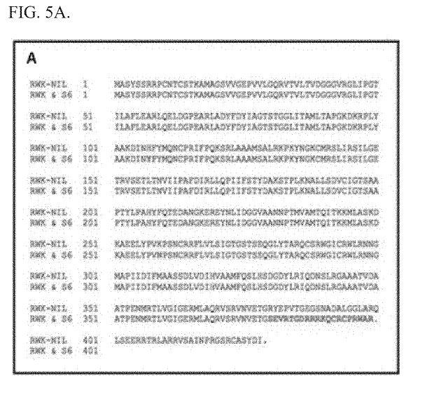

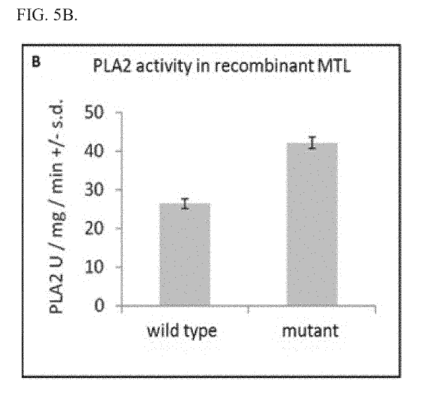

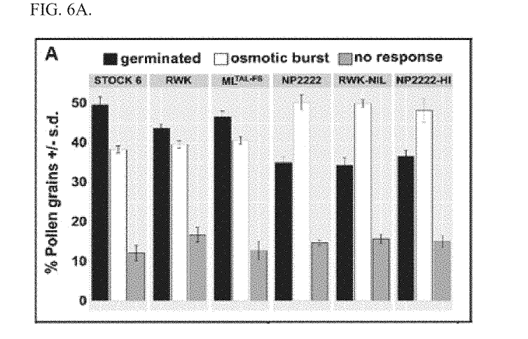

The frame-shift in mtl occurs at amino acid 380, leading to 20 altered amino acids followed by a premature stop codon which truncates the protein by 29 amino acids (FIG. 5a). The wild-type MTL protein was found in LS-MS profiles of RWK-NIL and NP2222 pollen, but was below the detection limit in three out of three RWK and 3 out of 3 NP2222-HI samples (Table 6). This demonstrates that even though there is mutant mtl transcript produced in pollen, the protein is not detected, confirming this is a loss of function mutation. Both mutant and wild type recombinant MTL proteins exhibited phospholipase activity in vitro in pPLAIIα-like fluorescent liposome cleavage assays (FIG. 5b). This demonstrates that the functional annotation of the MTL gene (i.e. that it codes for a phospholipase protein) is correct.

TABLE 6
Proteins off and on in NP2222 and NP2222-HI pollen samples, including MTL,
which is found in NP2222 but not NP2222-HI pollen.

| | | NP2222 | log2 | LFQ | NP2222-HI | | | |
|---|---|---|---|---|---|---|---|---|
| | rep | rep | rep | rep | rep | rep | | |
| | 1 | 2 | 3 | 1 | 2 | 3 | Majority protein ID | best BlastP match (S prot plants) |
| Absent | 23.3 | 23.3 | 23.4 | ND | ND | ND | GRMZM2G028905 | L-fucose alpha-1,3-D-xylosyltransferase |
| in | 22.9 | 24.1 | 23.9 | ND | ND | ND | GRMZM2G046743 | Lysine histidine transporter 1 |
| NP2222-HI | 23.2 | 23.1 | 23.2 | ND | ND | ND | GRMZM2G310362 | Polyadenylate-binding protein 5 |
| | 24.2 | 24.2 | 24.1 | ND | ND | ND | GRMZM2G130121 | Chaperone protein CIpB2, chloroplastic |
| | 23.5 | 23.5 | 23.6 | ND | ND | ND | GRMZM2G375807 | ABC transporter D; COMATOSE |
| | | | | | | | | Phospho-2-dehydro-3-deoxyheptonate aldolase |
| | 24.1 | 23.7 | 23.9 | ND | ND | ND | GRMZM2G396212 | 1 |
| | 23.7 | 23.8 | 23.9 | ND | ND | ND | GRMZM2G467907 | RNA-binding protein 47 |
| Absent | 23.8 | 23.8 | 23.9 | ND | ND | ND | GRMZM2G471240 | Matrilineal |
| in | ND | ND | ND | 23.9 | 23.8 | 23.9 | GRMZM2G013607 | Ferredoxin-6, chloroplastic |
| NP2222-HI | ND | ND | ND | 22.1 | 22.1 | 22.2 | GRMZM2G030971 | Phospholipase A I |
| | ND | ND | ND | 24.5 | 24.6 | 24 | GRMZM2G064967 | Mannan endo-1,4-beta-mannosidase |
| | ND | ND | ND | 24 | 24.3 | 24.2 | GRMZM2G143613 | F-box protein |
| | ND | ND | ND | 24.4 | 24.3 | 24.1 | GRMZM2G166906 | HOTHEAD (synth long-chain a-dicarboxylic |
| | | | | | | | | FAs) |
| | ND | ND | ND | 23.2 | 23.5 | 23.6 | GRMZM2G181259 | beta-D-xylosidase 2 |

*ND, Not detected

II. Pollen Germination and Localization Experiments

Full length functional reporter lines were used to characterize MTL localization. No signal was found in the pollen of NP2222 or NP2222+mtl-GFP/mtl-GFP. In contrast, NP2222+MTL-GFP/MTL-GFP pollen exhibited a strong signal in the cytoplasm of the two sperm cells. This signal was found in the stringy gamete cytoplasm within germinated pollen tubes. NP2222 embryo sacs fixed 18 hours after pollination with MTL-GFP pollen had signal in the area of the degenerating synergid consistent with that of SCs delivered during fertilization. This indicates MTL is part of the male germ unit that is deposited in the embryo sac after pollen tube burst. MTL-GFP but not mtl-GFP eliminated haploid induction in NP2222-HI (Table 3). Collectively these data indicate that MTL is a phospholipase specific to the SC cytoplasm, and that the frame-shift in mtl compromises MTL localization or stability in haploid inducer pollen.

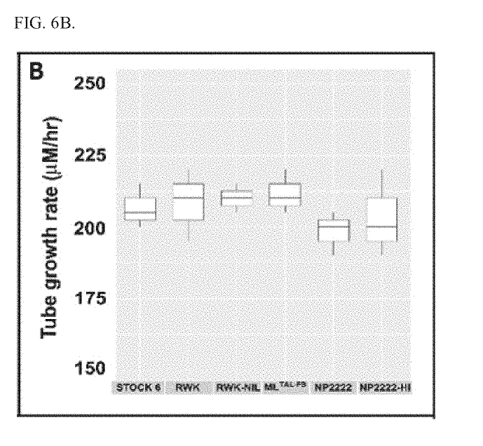

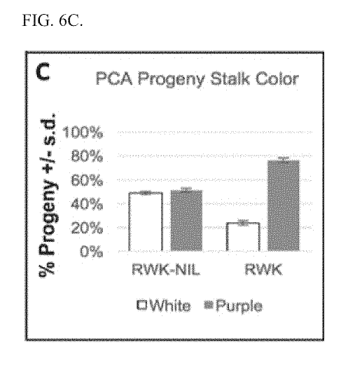

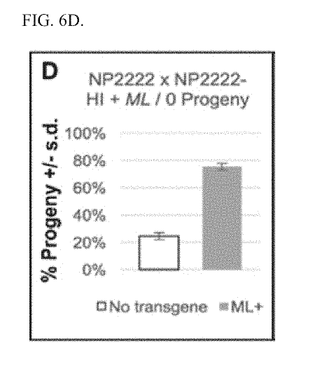

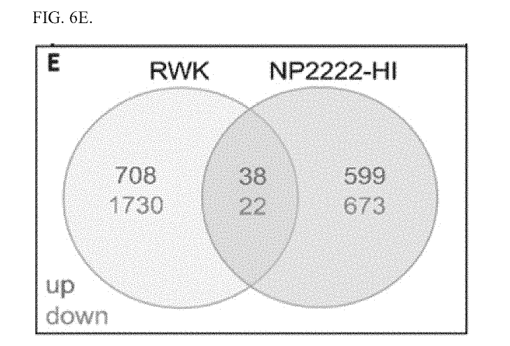

The identification of MTL as the causative gene in maize haploid induction permitted dissection of the pleiotropic phenotypes historically associated with the trait. Phospholipase mutations are associated with delayed pollen germination and tube growth (see Kim, H. J., et al. Endoplasmic reticulum- and golgi-localized phospholipase A2 plays critical roles in Arabidopsis pollen development and germination, PLANT CELL 23, 94-110 (2011)), but these were normal in RWK, Stock 6 and MTLT^{TAL-FS} lines (FIG. 6A, B). Ears pollinated with NP2222-HI and MTL^{TAL-FS} pollen exhibit ˜10-25% fertilization failure, and a pollen competition assay showed that RWK is subject to segregation distortion (SD) (FIG. 6C), consistent with prior reports (see Xu, X., et al. Gametophytic and zygotic selection leads to segregation distortion through in vivo induction of a maternal haploid in maize, J. EXP. BOT. 64, 1083-1096 (2013)). Crosses with hemizygous NP2222-HI+MTL/0 pollen produced a proportional bias towards MTL+progeny (FIG. 6D), indicating that inducer SD is attributable to mtl. Embryo abortion, a persistent byproduct of haploid induction linked to endosperm proliferation failure, occurred at similar rates in native and MTL^{TAL-FS} inducers (Table 4). Collectively these data implicate mtl in every reproductive defect associated with haploid induction. The two mechanisms typically proffered to explain haploid formation are single fertilization and post-zygotic genome elimination (see Sarkar, K. R. & Coe, E. H, A genetic analysis of the origin of maternal haploids in maize, GENETICS 54, 453-464 (1966); Zhang, Z., et al., Chromosome elimination and in vivo haploid production induced by Stock 6-derived inducer line in maize (Zea mays L), PLANT CELL REP. 27, 1851-1860 (2008); and Barret, P., Brinkmann, M., & Beckert, M., A major locus expressed in the male gametophyte with incomplete penetrance is responsible for in situ gynogenesis in maize, THEOR. APPL. GENET. 117, 581-594 (2008)). In the former, haploids result from fertilization of the central cell but not the egg, which subsequently develops via parthenogenesis. In the latter, double fertilization precedes male chromosome elimination. Clarifying the precise mechanism will require careful embryology after MTL^{TAL-FS} pollinations, along with quantitative data tracking the rare persistence of male DNA in maize haploids.

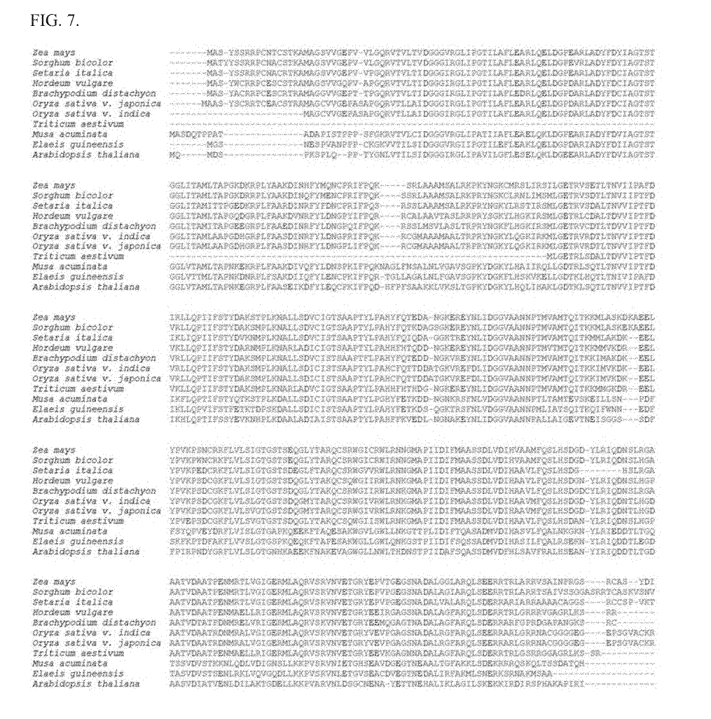

Haploid induction was recently engineered in Arabidopsis via manipulation of CENTROMERIC HISTONE3, which causes uniparental genome elimination through post-zygotic centromere imbalance between hybridized genomes. An attempt to replicate this in maize was successful (see Ravi, M. & Chan, S. W. L. Haploid plants produced by centromere-mediated genome elimination, NATURE 464, 615-618 (2010)), but this filing is the first instance of a haploid inducer system triggered by a cytoplasmic protein that does not bind chromatin. Thus, this work highlights the importance of non-nuclear sperm components in reproductive success and faithful genome transmittance. The conservation of MTL in the grasses (see FIG. 7), especially in rice where the closest homolog is pollen-specific and also found in sperm, suggests these findings will lead to the development of novel intra-specific haploid inducer lines in important crop plants.
III. Chemical Sprays to Induce de novo HI

After discovering that the MTL phospholipase (and specifically, the mtl loss-of-function frameshift mutation) triggers haploid induction, we tested whether phospholipase inhibitors, fatty acids, or other lipid compounds might act as chemical haploid inducers.

We have successfully achieved de novo haploid induction through application of various chemistries to flowers, silks, ears, tassels, and pollen. These chemistries are all in the class of lipids or phospholipase inhibitors. Table 7 outlines those chemistries and the haploid induction rates that resulted from their application. There is also formulation information and mode of application information in Table 7. From this table it is clear that a variety of lipid compounds induce haploids de novo when applied before, during, or after pollination to silks, which is stigmatic tissue specific to the maize plant, pollen, tassels (which contain male flowers containing pollen prior to pollen shed), and ears. Such compounds include mixed oils, fatty acids, fatty acid esters, phospholipids, and phospholipase inhibitors.

We tested many phospholipase inhibitors, including manoalide, a phospholipase inhibitor without any fatty acid chains. This chemistry was able to induce haploid formation de novo. With that result in hand, we started testing more compounds. We found that the most active compound for de novo induction is methyl alpha linolenyl fluorophosphonate (MALFP), a potent phospholipase inhibitor and inhibitor of other lipid modifying enzymes. This compound contains three linolenate fatty acid chains (eighteen carbons with three triple bonds) and a methyol group and fluorine atom at the head group position. Another highly active de novo haploid inducer compound is methyl arachidonyl fluorophosphonate (MAFP), another phospholipase inhibitor with a slightly different fatty acid chain structure (20 carbons and four double bonds). Without wishing to be bound by theory, MALFP and MAFP inhibit phospholipases by sitting in the fatty acid chain binding pocket, and in some cases catalyzing irreversible phosphorylation of the serine amino acid in the active site (see Lio Y. C., Reynolds L. J., Balsinde J., and Dennis E. A. Irreversible inhibition of Ca(2+)-independent phospholipase A2 by methyl arachidonyl fluorophosphonate. BIOCHIM BIOPHYS ACTA 1302:55-60 (1996)). Some fatty acids such as linoleic acid and linolenic acid can also non-competitively inhibit phospholipases by sitting in the fatty acid chain binding pocket without covalently modifying the active site, though the effect of this inhibition is weaker. Ballou, L. R., and Cheung, W. Y. Inhibition of human platelet phospholipase A2 activity by unsaturated fatty acids PROC NATL ACAD SCI 82 (2): 371-375 (1985)). These references may help to explain the reason for our findings that de novo haploid induction is not only triggered by strong inhibitors of phospholipases like MALFP and MAFP, but also by fatty acids esters including arachidonic acid methyl ester (AAME), which has 20 carbons and four double bonds (poly unsaturated 20 carbon fatty acid ester), as well as linoleic acid (LLE), which has 18 carbons and two double bonds (18:2), and linolenic acid (LNA), which has 18 carbons and three double bonds (18:3), and oleic acid ethyl ester (OAEE), and arachidonic acid (which has 20 carbons and four double bonds). Again it is thought that these unsaturated fatty acid chains, whether in their free fatty acid form, their ester form (which are synthesized in the lab and may be able to penetrate tissues) or in some other form, can act as inhibitors of phospholipases. While it is generally not recognized that the ester form of these compounds can inhibit phospholipases (see Ballou, supra), we find that such fatty acid methyl and ethyl esters can indeed lead to de novo haploid function. See Table 7. Of note, saturated fatty acids and derivatives, including stearic acid and palmitic acid ethyl ester, were tested. The prior art states that molecules that contain saturated fatty acid side chains do not or would not be able competitively inhibit a phospholipase by residing in its active site. See Ballou, supra. Such compounds were believed to lack the ability to inhibit phospholipases. However, we were surprisingly able to use molecules containing saturated fatty acid side chains to induce formation of haploids de novo. For example, palmitic acid ethyl ester (PAEE) induces haploids. See Table 7.

We also decided to test whether certain oils and phospholipids could trigger haploid induction de novo, including common plant oils. We found that one highly active de novo haploid inducer lipid mixture is corn oil, a natural plant extract that can be purchased from a grocery store. Another highly active lipid mixture is linseed oil that can be purchased from a hardware store. Canola oil, vegetable oil, peanut oil, sesame oil, or any other oil, when applied in the correct fashion, will work as well. Another highly active compound is 1,2-distearyl-sn-glycero-phosphatidylcholine (DSPC) (also known as 18:0 18:0 PC), a phospholipid with a phosphatidylcholine head group and a two 18-carbon saturated fatty acid chains. This common phospholipid is present in many biological materials because it is one of the core and most abundant components of phospholipid bilayers, which comprise the cellular membranes of all living things on the planet. Another haploid inducing compound is phosphatidylethanolamine with one stearyl and one oleoyl chain (also known as 1-stearyl-2-oleoyl-sn-phosphatidylethanolamine), another common phospholipid. Other phospholipids, as well as lyso-phospholipids and other triacylglycerides, diacylglycerols, lysophospholipids, triterpenoid esters, or glycerolipids will act as de novo haploid inducers. These oils and phospholipids are not commonly known to inhibit phospholipases, but they may be the source material for the generation of the byproducts of phospholipase activity, including the very fatty acids that inhibit phospholipases. The fact that these compounds act as de novo haploid inducer chemistries thus indicates these oils and phospholipids may inhibit phospholipases indirectly, or perhaps they are causing an imbalance or alteration in lipid or membrane composition of pollen leading to haploid induction. Either way, it is clear from this work that an enormous range of lipid-containing compounds, in addition to non-lipidic phospholipase inhibitors, when applied to flowers, can act as chemical inducers of haploid seed.

We also tested whether the buffers we used to apply these compounds to flowers were able to induce haploids by themselves. These buffers variably included certain surfactant blends and/or salt and/or DMSO (dimethyl sulfoxide) and/or other related additions. These buffers were not able to induce haploids, as can be seen in lines 121-123 of Table7. Also, de novo haploid induction without the addition of any compounds is extremely rare in maize, as can be seen from line 1. Two haploids were found among the 3,073 progeny tested (Kelliher, et al. Maternal haploids are preferentially induced by CENH3-tailswap transgenic complementation in maize, FRONTIERS IN PLANT SCIENCE 7: 414 (2016)). Thus, the control induction rate was found to be approximately 0.065%, which falls in the reported background range of gynogenic haploid induction (0.05-0.1%) (Chang, M.-T., and Coe, E. “Doubled haploids,” in Molecular Genetic Approaches to Maize Improvement, eds A. L. Kriz and B. A. Larkins (Heidelberg: Springer),127-142).

The mode of application of these various lipid and lipase inhibitor compounds can be quite variable and still produce haploids. One such mode of application is to dissolve the compounds in a salt solvent with additions of 1% DMSO. Another such solvent is a surfactant blend (Table 8). The tissue of application can also vary, as is evident from Tables 7, where application to both pollen and silks during pollination, as well as to tassels hours or days before pollination, can result in the formation of haploids. Furthermore, a wide variety of concentrations of the compounds can induce haploids, from 20 uM to 100 mM, or from 0.2 mg/mL up to 50 mg/mL. In fact, a much wider range than this is able to induce haploids, as can be seen in Table 7.

In two cases, we have also developed novel surfactant blends to help specific classes of lipids be able to emulsify and form a microemulsion (an aqueous-organic mixture with lipid droplet sizes smaller than a certain size—at least smaller than 10 microns in diameter and in many cases smaller than 1 micron in diameter). See Table 8 for the composition of the two surfactant blends. Blend “91” is ideally used with fatty acids at a concentration of 6.5 parts surfactant blend to 1 part fatty acid. However, multiple related concentrations and related surfactant blends could also work. Surfactant Blend “92” is ideally used with fatty acid esters, as well as oils, either in a pure form or as a triglyceride mixture. This is ideally blended at a ratio of 10 parts surfactant blend to 1 part ester or oil. Either of these blends or related blends of compounds may be suitable for proper dissolving or microemulsion construction with related lipid molecular classes outside of fatty acids, esters, and oils. These may include phospholipids, diacylglycerols, lysophospholipids, triterpenoid esters, or glycerolipids.

These surfactant blends are typically blended with the active ingredient and then mixed at a certain percentage with an aqueous buffer to make an emulsion and ideally a microemulsion. When making the emulsion, the percentage of the surfactant blend+Active ingredient in the aqueous buffer can be anywhere from 0.01-50%. The aqueous buffer can consist of any number of things. We have used 1×PBS+1% DML and PBS+50% DML, but other formulations work as well. “PBS” stands for phosphate-buffered saline; “DML” stands for dimethyl lactamide.

The use of these buffers alone was not able to lead to de novo haploid induction, as can be seen in Table 7, lines 121-123.

The mode of application of the compounds can take any number of forms including floral dip, floral injection, microinjection, pollen soaking, pollen misting, pollen spraying, floral misting, floral spraying, silk dousing, silk spraying, etc. We have most often used tassel, pollen, and silk spraying before, during, or after pollination. We have achieved de novo haploid formation with DSPC and linolenic acid when tassels were sprayed two days prior to pollination. We have also achieved de novo haploid formation with DSPC when tassels were sprayed just a few minutes prior to pollen collection and subsequent pollination. Similarly, we have achieved de novo haploid formation with DSPC when pollen was sprayed just before or during pollination, and also when silks were sprayed before, during, or after pollination, including up to two days before pollination and up to six hours after pollination.

We have also demonstrated de novo haploid formation when different applicators were used, including a cheap, plastic spray bottle that one can purchase at a convenience store, and a nebulizer, which is a medical device used to deliver medicines orally in the form of very small aqueous droplets. While the spray bottle typically produced droplet sizes of 50-150 microns in diameter, the nebulizer is able to generate droplet sizes of less than 10 microns. This is beneficial for application to pollen because pollen sizes range from 20-200 microns in diameter in most plants (approximately 70 microns in maize; approximately 50 microns in rice) and if one of the droplets from a typical spray bottle hits a grain, the droplet would be bigger than the grain. Pollen is extremely sensitive to moisture and osmotic shock. If the pollen grain comes into contact with a droplet of too large a size, that grain fails to germinate a successful, growing pollen tube. The same might be the case if the grain lands on a stigma or silk that is too wet—for instance, if that silk or stigma received too much of the lipid application (be it a microemulsion, or simply lipid droplets or micelles formed in an aqueous solution). If we applied more than 3 mL of lipid spray to the pollen or silks via spray bottle or nebulizer, we would often get very low seed set.

Therefore the mode of application is critical and in particular it is most important to try to apply as little volume of spray as possible to the pollen or stigma or silks or to the flower generally, and the best way to distribute the active ingredient, especially a lipid, with as little volume as possible is to make a microemulsion of that lipid in an aqueous buffer where the lipid droplet sizes is at the sub-micron level, and then to dispense that microemulsion with a nebulizer or similar device capable of making droplet sizes in the 1-2 micron range. This results in delivery of millions of droplets of active ingredient in “vapor” form to the relevant tissues, with the droplet size being >100,000× smaller volume than the pollen grain itself (if the droplet is 1 micron, and the pollen grain is 50 microns, then the diameter of the droplet is 50× smaller than the grain. And 50³=125,000, so the volume of the droplet is >105,000 times smaller than the pollen grain. With microemulsions, the lipid droplet size can range from approximately 20-1000 nm in diameter, so depending on the concentration of the surfactant blend plus active ingredient in the aqueous solution, one might have one or more lipid packets in each droplet delivered to each pollen grain or silk. Furthermore, considering some lipid type molecules or phospholipase inhibitors can be dissolved in an aqueous solution instead of being delivered as a microemulsion, we find that a wide variety of concentrations of the compounds can induce haploids, from 20 μg/mL to up to 50 mg/mL. Possibly higher concentrations will induce haploids de novo.

TABLE 8
Recipes for formulation 91 (“F91”) and formulation 92 (“F92”),
which help active ingredients such as fatty acids, oils, esters,
and phospholipids to be emulsified in very small lipid droplets.
Formulation 91 is a surfactant blend to be mixed with the fatty acid
optimally at 1 part fatty acid to six and a half parts formulation
91. The mixture is meant for creating emulsions with an aqueous buffer
at concentrations of 0.001 - 50%. Formulation 92 is also a surfactant
blend which can be mixed with esters or oils optimally at 1 part
active ingredient to 10 parts formulation 92, and then emulsified
in an aqueous buffer at concentrations of 0.001 - 50%.

| | Amount | |
|---|---|---|
| | (grams) | Chemical identity |
| Formulation 91 | | |
| Jeffsol AG1560 | 95.99 | butylene carbonate solvent |
| Emulpon CO360 | 24.02 | castor oil ethoxylate (36 mole EO) |
| Witconate | 24.08 | dodecyl (branched) benzene sulfonate, |
| P1220EH | | Ca salt. 60% solution in ethyl-hexanol |
| Agrimer AL-22 | 12.03 | poly(alkyl-pyrrolidone). Alkyl mainly C16 |
| | 156.12 | |
| Formulation 92 | | |
| Agnique AMD 3-L | 104 | dimethyl lactamide solvent |
| Witconic 1298 | 15.67 | dodecyl (linear) benzene sulfonic acid |
| soft | | |
| Surfonic T-5 | 12.2 | tallowamine ethoxylate (5 mole EO) |
| Surfonic T-15 | 24.21 | tallowamine ethoxylate (15 mole EO) |
| | 156.08 | |

IV. Increasing the HIR and Restoring Seed Set by Application of a Lipid Compound

Molecular profiling, including metabolomics and lipomics profiling, of haploid inducer and non-inducer pollen (comparatively) show that haploid inducer pollen is particularly deficient in certain types of lipid classes and overabundant in others. This deficiency is particularly pronounced in the 18 carbon chain class of lipid molecules, although it is also seen in 20 carbon chain classes. The deficiency is particularly pronounced with 18 carbon chain lipids with one and two double bonds (the so-called oleates and linoleates) and the overabundance was found particularly with the 18 carbon chain lipids with three double bonds (the so called linolenates). With respect to the types of lipids that are altered in haploid inducer pollen, it is very broad, and includes triglycerides, diacylglycerides, free fatty acids, lyso-phospholipids, and phospholipids. The changes in lipid content in haploid inducer pollen are also variable across different levels of fatty acid saturation. After seeing this data, we decided to apply lipid compounds flowers during pollination with haploid inducer pollen to see if simply adding certain types of lipids might impact the rate of haploid formation or the kernel count. We were particularly interested in applying compounds that had, in their molecular structure, some 18 or 20 carbon saturated fatty acid chains that were among those that were seen to be lower in abundance in haploid inducer pollen (e.g. oleate and linoleates).

Lipid applications can also lead to increases in the rate of haploid formation, seed set, and reductions in the frequency of kernel abortion, as is indicated in Tables 10-13. Table 10 shows nine separate, controlled experiments using the compound methyl alpha linolenyl fluorophosphonate (MALFP). Each experiment involved two ears crossed by the same haploid inducer male parent plant, on the same day, at nearly the exact same time (within 5 minutes of each other) from the exact same pollen population collected from the same male parent. These two ears either received a control buffer application, or a buffer plus active ingredient. Both the male and female test plants were grown next to each other under the same conditions in the same greenhouse. On average, and compared to control applications of buffer without the MALFP, when MALFP was applied the haploid induction rate increased by 3.6%, the number of kernels increased by nearly 42 per ear, and the number of total haploids recovered per ear increased by 11.8 haploids per ear, and the embryo abortion frequency decreased by 1.1% (Table 9). These all represent improvements for a haploid induction production scheme. Improvements were particularly apparent when formulation 91 was used as the surfactant blend to dissolve the MALFP. The mechanism by which these inhibitors are having an additive effect with mtl leading to higher induction rates could be that they are disrupting other phospholipases or lipid-modifying proteins.

The same type of data was collected for the fatty acid ethyl ester LLAEE, leading to an increase in the haploid induction rate of 2.4%, an increase of over 43 kernels formed per ear, an increase over 8 haploids per ear, and a decrease in the frequency of embryo abortion of 3.7%. Though many of these treatments involved different amounts of the active ingredient, on the majority of these experiments LLAEE led to a boost in these haploid formation and seed setting statistics. See Table 10.

The same type of data was collected for the fatty acid linoleic acid (LLA) (Table 11), leading to an increase in the haploid induction rate of 1.5%, an increase of 41 viable kernels per ear and 7.6 haploids per ear, and a decrease in the embryo abortion rate of 2.9%. This is the result of 22 separate experiments, again with each experiment consisting of one ear pollinated by the control buffer and one ear pollinated by the buffer plus active ingredient (LLE). It is interesting that the abundance of 18:2 is down in haploid inducer pollen (as shown by the lipomics data). Without wishing to be bound by theory, it is reasonable to hypothesize that this may cause or contribute to fertilization issues and embryo abortion. When we add these back via spray application of LLA and LLAEE, these molecules could be inhibiting phospholipases leading to higher HIR, and supplementing 18:2 leading to more kernels.

Finally, similar data was collected for MAFP, and similar results were seen, including a boost to the haploid induction rate of 1.9%, an increase in the number of haploids recovered per ear of 6.6 and the number of total kernels of 28, well as a decrease in the embryo abortion rate of 2.2% (Table 12). These compounds, as well as other lipids, can be used to boost the frequency of haploid embryo formation in haploid induction nurseries, and can also be used to increase seed set in the context of self-pollinating haploid inducer lines in order to increase more stock seed of those lines.

TABLE 9
MALFP effect on HIR, kernel set, kernel abortion, and overall haploids recovered.

| | | | Comparing MALFP to the control | | | |
|---|---|---|---|---|---|---|
| Experiment | MALFP | | Δ | | Δ | Δ k. a. |
| No. | Concentration | Buffer | Haploids | Δ HIR | kernels | rate |
| 1 | 2% | formulation 91 | +37 | +16.3% | −52 | +16.3% |
| 2 | 2% | formulation 91 | +25 | +9.4% | +101 | −4.4% |
| 3 | 2% | formulation 92 | −9 | −6.2% | +68 | −3.4% |
| 4 | 2% | formulation 92 | +71 | +29.8% | +86 | −21.0% |
| 5 | 10% | formulation 92 | −20 | −7.9% | −38 | −2.4% |
| 6 | 14% | formulation 91 | +2 | −0.5% | −18 | +19.9% |
| 7 | 14% | formulation 91 | +11 | +3.5% | +70 | −19.2% |
| 8 | 20% | formulation 92 | −9 | −6.0% | +89 | −4.0% |
| 9 | 20% | formulation 92 | −9 | −7.6% | +69 | −4.3% |
| | | | | | | k. a. |
| | | AVERAGE/EAR | haploids | HIR | kernels | rate |
| | | MAFP | 34.6 | 17.1% | 238.8 | 32.4% |
| | | control | 22.8 | 13.5% | 197.3 | 33.5% |
| | | MAFP vs control | +11.8 | +3.6% | +41.5 | −1.1% |

TABLE 10
LLAEE effect on HIR, kernel set, kernel abortion, and overall haploids recovered.

| | | Comparing LLAEE to the control | | |
|---|---|---|---|---|
| Experiment | LLAEE | Δ | Δ | Δ k. a. |

| No. | Concentration | Buffer | Haploids | Δ HIR | kernels | rate |
|---|---|---|---|---|---|---|
| 1 | 2% | formulation 92 | +7 | +11.3% | −29 | −2.4% |
| 2 | 5% | formulation 92 | +3 | +1.7% | +12 | +1.8% |
| 3 | 20% | formulation 92 | +10 | +1.7% | +117 | −14.5% |
| 4 | 0.3 mg/mL | PBS + 1%DMSO | +19 | +6.5% | +97 | +11.1% |
| 5 | 0.3 mg/mL | PBS + 1%DMSO | +4 | +1.8% | +14 | −8.2% |
| 6 | 0.3 mg/mL | PBS + 1%DMSO | −5 | +11.7% | −117 | −11.0% |
| 7 | 1 mg/mL | PBS + 1%DMSO | +20 | +6.8% | +44 | −5.3% |
| 8 | 1 mg/mL | PBS + 1%DMSO | +14 | +7.7% | +67 | +0.2% |
| 9 | 1 mg/mL | PBS + 1%DMSO | +8 | −1.7% | +87 | +5.6% |
| 10 | 3 mg/mL | PBS + 1%DMSO | = | −6.7% | +99 | −17.0% |
| 11 | 3 mg/mL | PBS + 1%DMSO | +13 | +8.8% | −29 | −0.6% |
| 12 | 3 mg/mL | PBS + 1%DMSO | −1 | −5.2% | +110 | −9.8% |
| 13 | 3 mg/mL | PBS + 1%DMSO | +20 | +6.7% | +17 | −0.4% |
| 14 | 3 mg/mL | PBS + 1%DMSO | −7 | +3.0% | +24 | −15.3% |
| 15 | 10 mg/mL | PBS + 1%DMSO | +2 | −0.6% | +31 | −1.5% |
| 16 | 10 mg/mL | PBS + 1%DMSO | +9 | = | +51 | −14.2% |
| 17 | 10 mg/mL | PBS + 1%DMSO | +4 | +2% | −2 | −1.0% |
| 18 | 20 mg/mL | PBS + 1%DMSO | +4 | +0.9% | +21 | +4.7% |
| 19 | 30 mg/mL | PBS + 1%DMSO | +8 | −1.1% | +90 | −6.5% |
| 20 | 30 mg/mL | PBS + 1%DMSO | +5 | +6.6% | −64 | +10.7% |
| 21 | 30 mg/mL | PBS + 1%DMSO | +22 | +0.8% | +165 | −6.2% |
| | | | | | | k. a. |
| | | AVERAGE/EAR | haploids | HIR | kernels | rate |
| | | LLAEE | 23.1 | 13.0% | 194.1 | 29.9% |
| | | control | 14.9 | 10.6% | 150.8 | 33.6% |
| | | LLAEE vs control | +8.2 | +2.4% | +43.3 | −3.7% |

TABLE 11
LLA effect on HIR, kernel set, kernel abortion, and overall haploids recovered.

| | | | Comparing Linoleic acid (LLA) to the control | | | |
|---|---|---|---|---|---|---|
| Experiment | LLA | | | | Δ | Δ k. a. |
| No. | Concentration | Buffer | Δ Haploids | Δ HIR | kernels | rate |
| 1 | 7% | Formulation 91 | −9 | −2.7% | −9 | +7.6% |
| 2 | 0.1 mg/mL | PBS + 1%DMSO | +26 | +2.2% | +134 | +14.3% |
| 3 | 0.1 mg/mL | PBS + 1%DMSO | −18 | +10.2% | −122 | +9.2% |
| 4 | 0.1 mg/mL | PBS + 1%DMSO | +16 | +8.0% | +8 | +15.4% |
| 5 | 0.5 mg/mL | PBS + 1%DMSO | +12 | +0.2% | +108 | −9.4% |
| 6 | 0.5 mg/mL | PBS + 1%DMSO | +9 | +3.2% | +19 | −3.4% |
| 7 | 0.5 mg/mL | PBS + 1%DMSO | −3 | −0.5% | −14 | +2.3% |
| 8 | 1 mg/mL | PBS + 1%DMSO | +14 | +2.2% | +132 | −15.6% |
| 9 | 1 mg/mL | PBS + 1%DMSO | +9 | +1.7% | +87 | −0.7% |
| 10 | 5 mg/mL | PBS + 1%DMSO | +23 | +4.9% | +112 | −6.9% |
| 11 | 5 mg/mL | PBS + 1%DMSO | −1 | −3.1% | +53 | −5.5% |
| 12 | 5 mg/mL | PBS + 1%DMSO | −4 | −0.8% | −18 | +3.7% |
| 13 | 5 mg/mL | PBS + 1%DMSO | +10 | +1.6% | +61 | −3.0% |
| 14 | 10 mg/mL | PBS + 30% DML | +24 | +3.2% | +80 | −7.5% |
| 15 | 10 mg/mL | PBS + 30% DML | +9 | +1.4% | +44 | +2.2% |
| 16 | 10 mg/mL | PBS + 30% DML | −2 | −3.1% | +32 | −0.8% |
| 17 | 10 mg/mL | PBS + 30% DML | −5 | −2.6% | −49 | −0.2% |
| 18 | 10 mg/mL | PBS + 30% DML | +12 | +9.1% | +87 | −6.0% |
| 19 | 25 mg/mL | PBS + 1%DMSO | −15 | +3.7% | −92 | −5.1% |
| 20 | 25 mg/mL | PBS + 1%DMSO | +3 | +6.0% | −59 | +0.7% |
| 21 | 25 mg/mL | PBS + 1%DMSO | +21 | +2.5% | +97 | −11.8% |
| 22 | 25 mg/mL | PBS + 50% DML | +20 | +10.7% | +107 | −11.7% |
| | | AVERAGE/EAR | haploids | HIR | kernels | k. a. rate |
| | | LLAEE | 27.9 | 15.4% | 194.6 | 33.0% |
| | | control | 20.3 | 13.9% | 156.3 | 35.9% |
| | | LLAEE vs control | +7.6 | +1.5% | +41.7 | −2.9% |

TABLE 12
MAFP effect on HIR, kernel set, kernel abortion, and overall haploids recovered.

| Experiment | MAFP | | Comparing MAFP to the control | | | |
|---|---|---|---|---|---|---|
| No. | Concentration | Buffer | Δ Haploids | Δ HIR | Δ kernels | Δ k. a. rate |
| 1 | 20 ug/mL | PBS + 1%DMSO | +2 | +9.3% | −20 | +7.4% |
| 2 | 20 ug/mL | PBS + 1%DMSO | −4 | +0.6% | −27 | −5.9% |
| 3 | 20 ug/mL | PBS + 1%DMSO | +4 | −1.9% | +65 | +1.7% |
| 4 | 20 ug/mL | PBS + 1%DMSO | = | −1.4% | +33 | −0.6% |
| 5 | 50 ug/mL | PBS + 1%DMSO | +16 | +6.8% | +38 | −4.4% |
| 6 | 50 ug/mL | PBS + 1%DMSO | +12 | +4.6% | +60 | −8.8% |
| 7 | 50 ug/mL | PBS + 1%DMSO | +12 | +4.8% | +40 | −2.0% |
| 8 | 50 ug/mL | PBS + 1%DMSO | +17 | +10.5% | +18 | −7.7% |
| 9 | 50 ug/mL | PBS + 1%DMSO | −8 | −1.6% | −35 | +6.4% |
| 10 | 50 ug/mL | PBS + 1%DMSO | = | +9.1% | −86 | +6.1% |
| 11 | 2% | formulation 92 | +8 | = | +109 | −1.2% |
| 12 | 2% | formulation 92 | −1 | −2.4% | +26 | −10.9% |
| 13 | 10% | formulation 92 | +8 | +0.8% | +44 | +0.7% |
| 14 | 10% | formulation 92 | −5 | −6.9% | −7 | −2.1% |
| 15 | 20% | formulation 92 | −10 | −0.9% | +90 | −6.7% |
| 16 | 20% | formulation 92 | = | −0.8% | +10 | −5.8% |
| | | AVERAGE/EAR | haploids | HIR | kernels | k. a. rate |
| | | MAFP | 23.6 | 14.6% | 172.5 | 34.6% |
| | | control | 0 | 12.7% | 144.4 | 36.8% |
| | | MAFP vs control | +6.6 | +1.9% | +28.1 | −2.2% |

V. Mutagenesis/Knockouts of PLA

In an effort to alter the haploid induction rate or decrease the embryo abortion rate during haploid induction crosses, we created or obtained several mutant lines by several methods, including GM RNAi lines, TILLING lines, CRISPR lines, and TALEN lines. First, we sought evidence that targeted mutagenesis of pPLAIIα is a viable strategy to create new haploid inducer lines. Therefore, we tested both CRISPR/CAS9 and TALEN maize targeted mutation strategy aimed at the same sequence that contains the frame-shift in the mutant haploid inducer allele. This led to the generation of lines with novel mutations, which we tested for haploid induction.

There are three key components to the CRISPR process. See U.S. Pat. No. 8,697,359 B1, incorporated herein by reference in its entirety. The first key component is the target sequence. The second is the Cas9, which is the endonuclease. The third key component is the guide RNA (“gRNA”), which is complementary to the target sequence and is responsible for recruiting Cas9 to the desired location. The target sequence is 18 to 20 bp long, and optimally should be sitting just 5′ to a protospacer adjacent motif (“PAM”) in the plant genome. For Cas9 from Streptococcus pyogenes, the PAM sequence should be 5′-NGG-3′. Transcription of the gRNA can be driven by the Pol III promoter U3 (RNA starts with an A) or U6 (RNA starts with a G). The gRNA should carry target sequence at the 5′ end right after the A (U3) or G (U6). Cas9 will generate a double-stranded break (“DSB”) at the target sequence three base pairs 5′ to the PAM sequence. The amino acid sequence of Cas9 is the same as Cas9 from Streptococcus pyogenes strain SF370, with two amino acid changes, L1164V and I1179V in the PI domain (1099-1368) in NUC lobe. Cas9 activity has been demonstrated in transformation experiments to have approximately a 90% mutation frequency of tested target sequence in corn. Generally, it is advisable to identify multiple candidate PAMs and target sequences in the target region, then look for the best one by seeing which of the sequences is unique in the genome of the target. The target plant is maize, rice, or any monocot plant.

This strategy was followed to identify CRISPR target sequences that overlap with the existing frame-shift mutation. The precise cut site is just two base pairs away from the insertion point in the frame-shift. Constructs containing both the Cas9 and the gRNA were transformed into maize plants. Generally, biallelic or homozygous mutant plants are recoverable from the multiple events generated, but heterozygous mutant plants are also useful. The heterozygous plants were selfed, then the T1 seed was grown up, screened for homozygosity of the mutation, and outcrossed. Homozygous or biallelic mutant T0 transformants were simply selfed and outcrossed to untransformed NP2222. All outcrossed embryos were isolated for ploidy analysis to find haploids.

Three different targeted mutagenesis constructs created: CRISPR/CAS9 I, CRISPR/CAS9 II, and TALEN. The difference between CRISPR/CAS9 I and II is minor. The target site locus for all three constructs was the same region where the frame-shift was found in haploid inducer lines. For the CRISPR constructs, the guide RNA sequence starts at nucleotide +1560: -GTCAACGTGGAGACAGGG- (i.e., SEQ ID NO: 20). The —AGG— PAM site of SEQ ID NO: 20 is underlined and italicized. The four basepair insertion in haploid inducer lines is at that exact site, at nucleotide +1576. After transformation, several different CRISPR I events (comprising the expression construct found in SEQ ID NO: 34), CRISPR II events (comprising the expression construct found in SEQ ID NO: 36), and TALEN events (comprising the expression construct found in SEQ ID NO: 35) were selected, grown to maturity, and set viable seed. In the T0 generation, we performed PCR at the target site and sequenced the PCR products after sub-cloning. We identified many unique mutations amongst those events (and many of the events were chimeras or had multiple alleles).

Many plants were chimeric, as evidenced by multiple different sequences appearing in the T1 generation. After T0 self-pollination, the T1 plants segregated 1:2:1 for the target mutagenesis construct, and many had novel mutations at the target locus in either a biallelic or homozygous state. We screened seedlings at the DNA level using TAQMAN markers, identified the biallelics that lacked the Cas9 or TALEN transgenes, and performed PCR sequencing to produce PCR product reading basepairs +1494 to +1691 in the GRMZM2G471240 gene sequence. We then tested homozygous mutants for haploid induction capacity. See SEQ ID NOs: 9-19 & 42-44 for the sequences of the new T1 plants at the mtl gene.

The HIR was measured for the putative new lines. See Table 4, above. This HIR data is from crosses where the male was a putative haploid inducer line and the female was our standard inbred transformation line NP2222. The putative haploid inducer lines were created using either TALEN- or CRISPR/CAS9-mediated targeted mutation of the pPLAIIα locus. Among those shown here, there are eleven different putative inducer plants comprising eight different events from three distinct transformation constructs. Event 39A was a TALEN event. Events 18A and 27A were CRISPR events. The latter was a chimera as a T0 plant, and after it was self-pollinated, multiple mutations were found in the T1 population, including “biallelic” plants (by biallelic, we mean that when we sequenced the region of pPLAIIα that was mutated, we found two different novel alleles—such that it is clear that both wild type copies of the gene had been mutated, but they were mutated differently, so there are two novel alleles). Each of these eleven individual plants thus had distinct combinations of mutations in pPLAIIα. What they all had in common is that none of the eleven plants had a wild type copy of pPLAIIα. Therefore, these are all “homozygous mutant” for the pPLAIIα gene. The mutations were all frameshifts in exon 4, mimicking the original mutation in the native haploid inducer lines. Using these five plants as males, we crossed onto either one or several female ears, generating thousands of embryos. We dissected and did ploidy analysis on those progeny and discovered that each of the progeny sets had at least 3.98% haploids with a maximum of 12.5% haploids. This demonstrates that generating mutations in pPLAIIα will lead to haploid induction. We think that other types of mutations, besides frameshifts, will also lead to haploid induction. Those mutations could be anywhere in the gene, and they could be point mutations or insertions or deletions or other types of mutations.

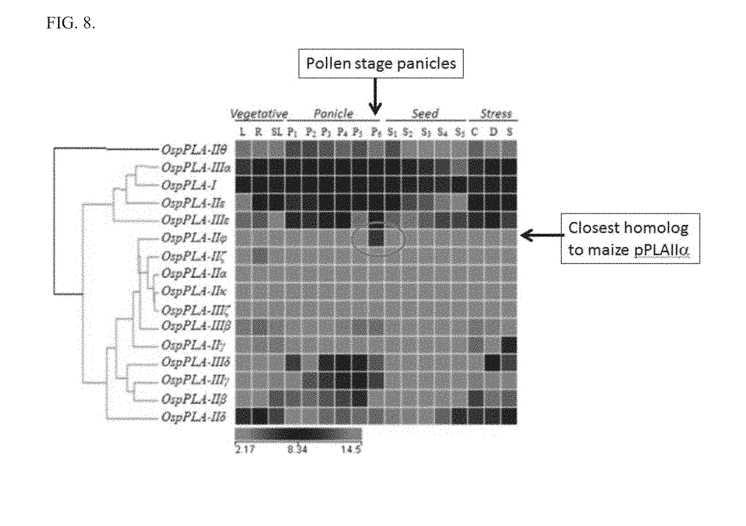

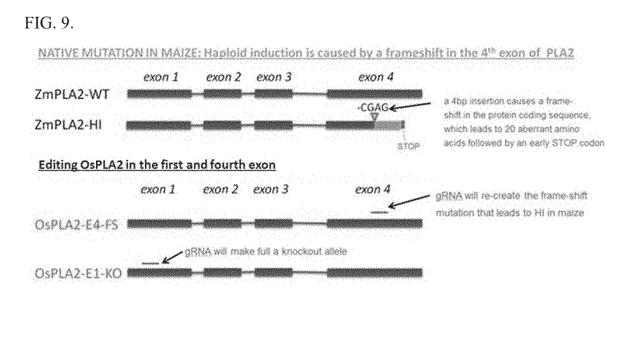

RNAi was also used to generate haploid inducer lines. For the RNAi, two hairpin constructs were made; one mapping to the border between exon 1 and 2, and the other mapping to exon 4 (FIGS. 8 and 9). The constructs were transformed into wild-type and the T0 plants were selfed. The T1 seed from three events per construct were grown, screened for homozygosity of the transgene, and outcrossed onto several ears as tests for haploid induction. After examining over 1500 kernels from these outcrosses, we found both events induce haploids at a rate of approximately 1% to 2%. The highest rate of haploid induction obtained on a single ear was 4.3%. That ear had about 300 kernels, so we can conclude that the embryo abortion rate was also lower than a typical high-inducer line. This work demonstrates than an RNAi+GM strategy can be used to create new haploid inducer lines in otherwise-typically wild-type lines by altering the expression of pPLAIIα.

The TILLING mutagenesis method was also used to create and identify the phospholipase mutations and maize of the present invention. Publications describing TILLING are available for crop plants such as rice: Till et al., BMC Plant Biology 7:19 (2007), tomato: Rigola et al. PLOS ONE Mar. 13, 2009, and maize: Till et al. BMC Plant Biol. 2004 Jul. 28;4:12 (2004), all of which are incorporated herein by reference. In the basic TILLING methodology, plant material, such as seed, is subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest.

Any cultivar of maize having at least one phospholipase gene with substantial homology to SEQ ID NO: 1 may be used in accordance with the present invention. As used herein, “substantial homology” means that the DNA sequence of the gene is sufficiently similar to SEQ ID NO: 1 at the nucleotide level to code for the equivalent protein as SEQ ID NO: 1, allowing for allelic differences between cultivars. In accordance with one aspect of an exemplary embodiment of the invention, “substantial homology” may be present when the homology between the phospholipase gene and SEQ ID NO: 1 is as low as about 85%, provided that the homology in the conserved regions of the gene is higher (e.g., at least about 90%). Preferably, the percent identity in the coding region is 85-90%, more preferably 90-95%, and optimally, it is above 95%. One of skill in the art may prefer a maize cultivar having commercial popularity or one having specific desired characteristics in which to create the phospholipase-mutated maize. Alternatively, one of skill in the art may prefer a maize cultivar having few polymorphisms, such as an in-bred cultivar, in order to facilitate screening for mutations within the phospholipase loci.

In accordance with one aspect of an exemplary embodiment of the present invention, seeds from rice and maize were mutagenized and then grown into M1 plants. The M1 plants were then allowed to self-pollinate and seeds from the M1 plant were grown into M2 plants, which were then screened for mutations in their phospholipase locus. While M1 plants may be screened for mutations, an advantage of screening the M2 plants is that all somatic mutations correspond to the germline mutations. One of skill in the art would recognize that a variety of maize plant materials, including, but not limited to, seeds, pollen, plant tissue or plant cells, may be mutagenized in order to create the phospholipase-mutated maize of the present invention. However, the type of plant material mutagenized may affect when the plant DNA is screened for mutations. For example, when pollen is subjected to mutagenesis prior to pollination of a non-mutagenized plant, the seeds resulting from that pollination are grown into M1 plants. Every cell of the M1 plants will contain mutations created in the pollen, thus these M1 plants may then be screened for phospholipase mutations instead of waiting until the M2 generation.

Mutagens that create primarily point mutations and short deletions, insertions, transversions, and or transitions (about 1 to about 5 nucleotides), such as chemical mutagens or radiation, may be used to create the mutations of the present invention. Mutagens conforming with the method of the present invention include, but are not limited to, ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino]acridine dihydrochloride (ICR-170), and formaldehyde. Spontaneous mutations in the nucleolar organizing region (“NOR”) that may not have been directly caused by the mutagen can also be identified in accordance with various embodiments of the present invention.

Any suitable method of plant DNA preparation now known or hereafter devised may be used to prepare the maize plant DNA for phospholipase mutation screening. For example, see Chen and Ronald, Plant Molecular Biology Reporter 17:53-57, 1999; Stewart and Via, Bio Techniques 14:748-749, 1993. Additionally, several commercial kits are available, including kits from Qiagen (Valencia, Calif.) and Qbiogene (Carlsbad, Calif.).

In accordance with one aspect of an exemplary embodiment of the invention, DNA samples from individual maize plants are prepared and then pooled in order to expedite screening for mutations in phospholipase of the entire population of plants originating from the mutagenized plant tissue. The size of the pooled group may be dependent upon the sensitivity of the screening method used. In accordance with one aspect of an exemplary embodiment of the invention, groups of four or more individual maize plants are pooled.

In accordance with another aspect of an exemplary embodiment, after the DNA samples are pooled, the pools are subjected to phospholipase sequence-specific amplification techniques, such as Polymerase Chain Reaction (PCR). For a general overview of PCR, see PCR Protocols: A Guide to Methods and Applications (Innis, Gelfand, Sninsky, J., and White, eds.), Academic Press, San Diego, 1990, which is incorporated herein by reference. Any primer specific to the phospholipase locus or the sequences immediately adjacent to the phospholipase locus may be utilized to amplify the phospholipase sequences within the pooled DNA sample. Preferably, the primer is designed to amplify the regions of the phospholipase locus where useful mutations are most likely to arise. Most preferably, the primer is designed to detect mutations in the coding region of the phospholipase gene. Additionally, it is preferable for the primer to avoid known polymorphic sites in order to ease screening for point mutations. To facilitate detection of PCR products on a gel, the PCR primer may be labeled using any conventional or hereafter devised labeling method.

In accordance with one aspect of an exemplary embodiment of the invention, the PCR amplification products may be screened for phospholipase mutations using any method that identifies nucleotide differences between wild type and mutant sequences. These may include, without limitation, sequencing, denaturing high pressure liquid chromatography (dHPLC), constant denaturant capillary electrophoresis (CDCE), temperature gradient capillary electrophoresis (TGCE) (see Li et al., Electrophoresis 23 (10):1499-1511, 2002), or by fragmentation using enzymatic cleavage, such as used in the high throughput method described by Colbert et al., Plant Physiology 126:480-484, 2001. Preferably, the PCR amplification products are incubated with an endonuclease that preferentially cleaves mismatches in heteroduplexes between wild type and mutant sequences. In accordance with another aspect of an exemplary embodiment, cleavage products are electrophoresed using an automated sequencing gel apparatus, and gel images are analyzed with the aid of a standard commercial image-processing program.

The present inventors have determined that to achieve haploid induction in maize, mutations that alter phospholipase function are desirable. Preferred mutations include missense, nonsense and splice junction changes, including mutations that prematurely truncate the translation of the phospholipase protein from messenger RNA, such as those mutations that create a stop codon within the coding regions of the phospholipase gene. Such mutations include insertions, repeat sequences, modified open reading frames (ORFs) and, most preferably, point mutations.

In accordance with yet another aspect of an exemplary embodiment of the invention, once an M2 plant having a mutated phospholipase sequence is identified, the mutations are analyzed to determine its effect on the expression, translation, and/or activity of the protein. In accordance with one exemplary embodiment, the phospholipase fragment containing the mutation is sequenced, using standard sequencing techniques, in order to determine the exact location of the mutation in relation to the overall phospholipase sequence. Each mutation is evaluated in order to predict its impact on protein function (i.e., completely tolerated to loss-of-function) using bioinformatics tools such as SIFT (Sorting Intolerant from Tolerant; Ng et al., Nucleic Acids Research 31:3812-3814, 2003), PSSM (Position-Specific Scoring Matrix; Henikoff and Henikoff, Computer Applications in the Biosciences 12:135-143, 1996) and PARSESNP (Taylor and Greene, Nucleic Acids Research 31:3808-3811, 2003). For example, a SIFT score that is less than 0.05 and a large change in PSSM score (e.g., roughly 10 or above) indicate a mutation that is likely to have a deleterious effect on protein function.

In accordance with a further aspect of an exemplary embodiment, if the initial assessment of a mutation in an M2 plant indicates it to be of a useful nature and in a useful position within the phospholipase gene, then further phenotypic analysis of the maize plant containing that mutation is pursued. First, the M2 plant is backcrossed or outcrossed twice to create a BC1 plant in order to eliminate background mutations. Then the backcrossed or outcrossed BC1 plant is self-pollinated in order to create a BC1F2 plant that is homozygous for the phospholipase mutation.

Several physical characteristics of these homozygous phospholipase mutant plants are assessed to determine if the mutation results in a useful phenotypic change in the maize. Mutant phospholipase maize are evaluated for haploid induction compared to normal (e.g., wild type) parental maize or to wild type sibling control maize. Table 13 shows novel mutations obtained by TILLING.

TABLE 13
Novel pPLAIIα Mutations Obtained by TILLING & their HIR. The nucleotide
change column represents the position from the start of the cDNA sequence (SEQUENCE
No. 1), and the changed nucleotide is capitalized within its codon context. The amino acid
change is then indicated followed by the impact of that change (Tolerated or Not Tolerated).
Of the two alleles that were not tolerated, one induced haploids at a rate of 1.04% (3/288).

| | | | AA | | | | PA | |
|---|---|---|---|---|---|---|---|---|
| Line | Nucleotide change | Exon | change | Tolerated? | Diploids | Haploids | confirmed | HR |
| 1139 | bp + 128 tGt/tAt | 1 | C13Y | Yes | 389 | 0 | 0 | 0.00% |
| 3594 | bp + 167 cCc/cTc | 1 | P26L | Yes | 381 | 0 | 0 | 0.00% |
| 0505 | bp + 431 ccg/cTg | 1 | P114L | No | 235 | 0 | 0 | 0.00% |
| 2658 | bp + 718 Gcg/Acg | 4 | A237T | Yes | 379 | 0 | 0 | 0.00% |
| 1983 | bp + 1077 atG/atA | 4 | M356I | No | 285 | 3 | 3 | 1.04% |
| 2732 | bp + 1163 aCt/aTt | 4 | T385I | Yes | 383 | 0 | 0 | 0.00% |
| 2414 | bp + 1226 aGa/aAa | 4 | R406K | Yes | 392 | 0 | 0 | 0.00% |

The nomenclature used in the Table 13 indicates the wild type nucleotide or amino acid, followed by its position according to the referenced SEQ ID N01, followed by the changed nucleotide or amino acid at that position using standard genetic code terminology.

For maize, TILLING the maize pPLAIIα gene generates new alleles which have low rates of haploid induction. This enables the creation of an allelic series, including knock-outs, of GRMZM2G471240. The sequence of two segments of this gene (maximum 1.5 kb, which equals 20 amplicons per gene) are screened for mutations. These sequences included the genomic sequence including introns, plus the predicted cDNA sequence and coding sequences for the two splice variants, elevant and unique amplicon sequences are designed based on those sequences, and mutation screening is performed in an existing bulked-M2 corn population. The identified mutants are characterized in terms of DNA sequence and consequences on translated protein sequence. The M3 seed is grown and selfed to generate M4 lines with putative mutant homozygous individuals segregating. These individuals are identified by PCR sequencing and outcrossed and selfed to test for these mutant lines' ability to induce haploids.

To execute the test crosses, the new lines are grown alongside a marker line which is homozygous recessive for a non-lethal color marker gene. Reciprocal crosses are used to test the specificity of induction to male vs. female transmission by evaluating the resulting plants for haploids, which exhibit the color phenotype. Positive hits are confirmed by the ploidy analysis as described above.

Individuals that are homozygous for the SNP mutations were crossed as males to the marker line female and led to the formation of a low rate of haploids in some instances (see Table 18). Positive hits are confirmed by the ploidy analysis as described above. In particular, a line that led to haploid formation had a G to A mutation at base pair 1077 of the cDNA sequence. This mutation causes an amino acid substitution of a methionine (M) to an isoleucine (I) at amino acid 356. This is a non-conservative amino acid change that may disrupt the protein's activity leading to the formation of low rate of haploids. Among 288 progeny tested, we found three haploids, for an induction rate of 1% (3/288).
VI. Creating Haploid Inducing Lines in Rice

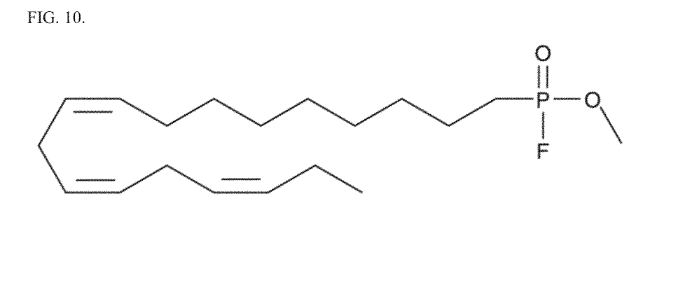

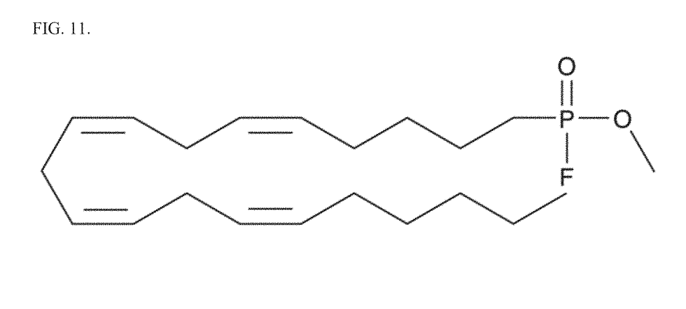

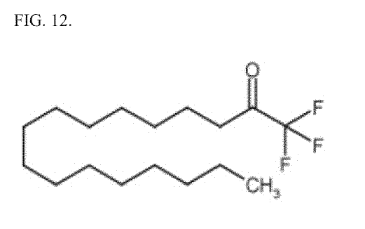

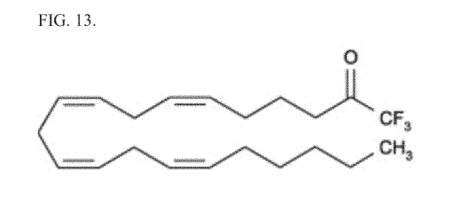

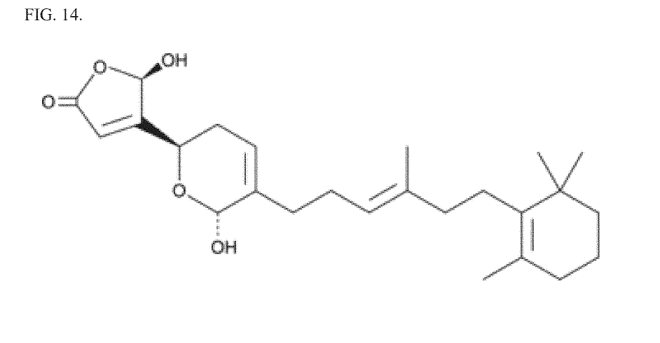

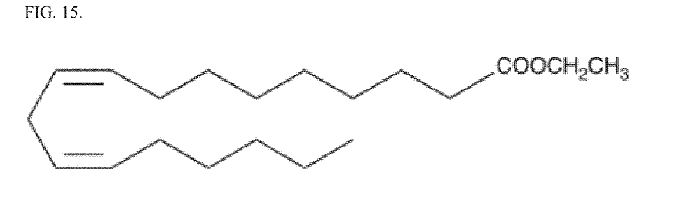

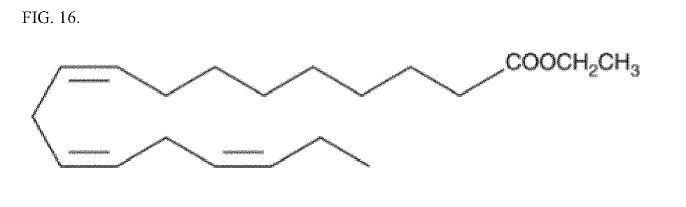

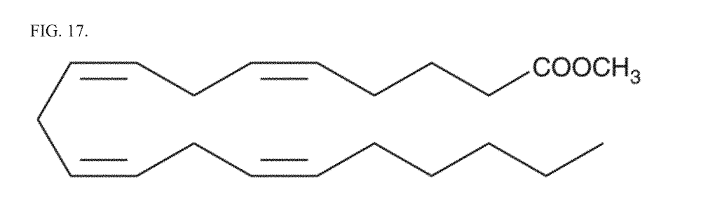

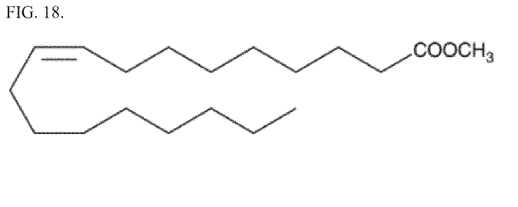

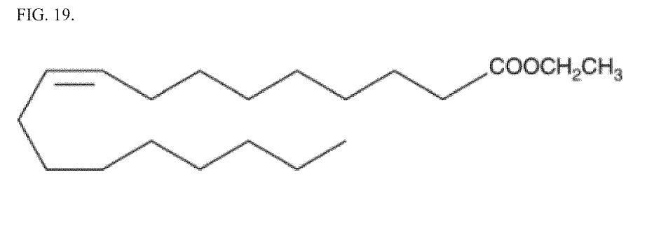

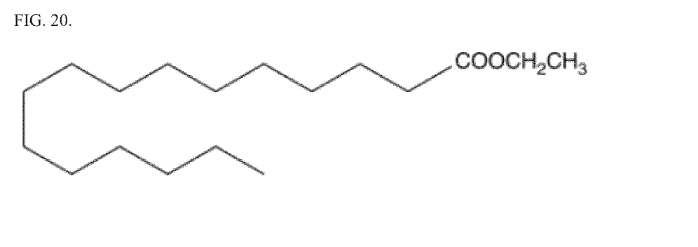

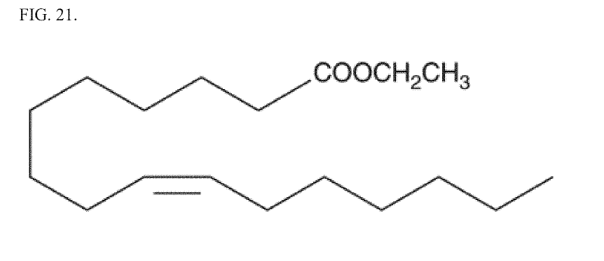

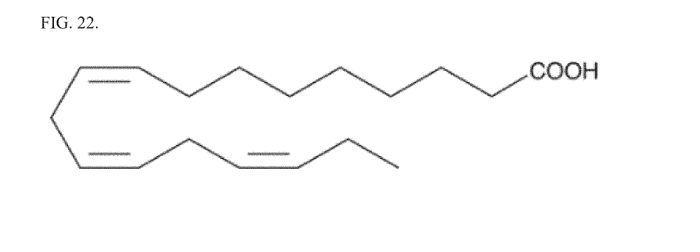

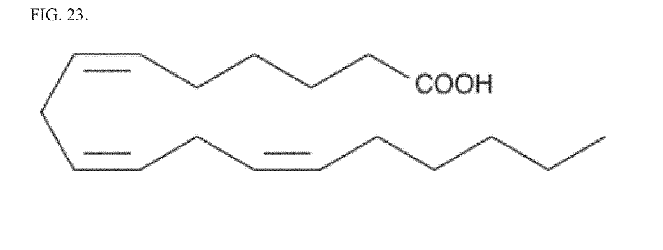

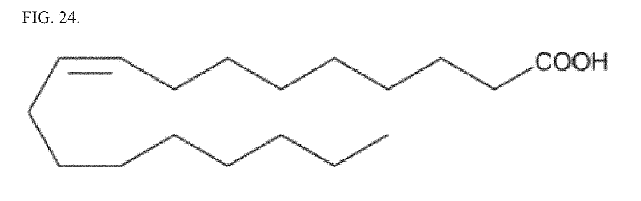

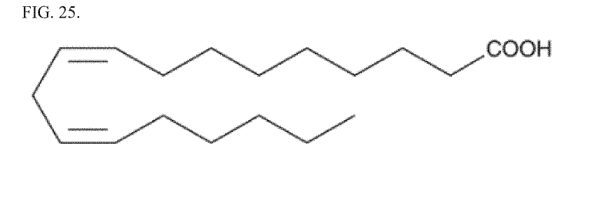

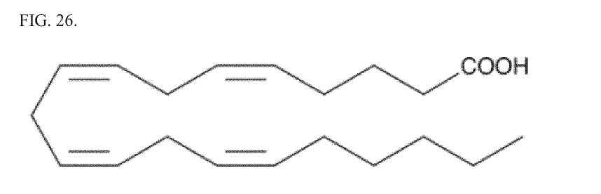

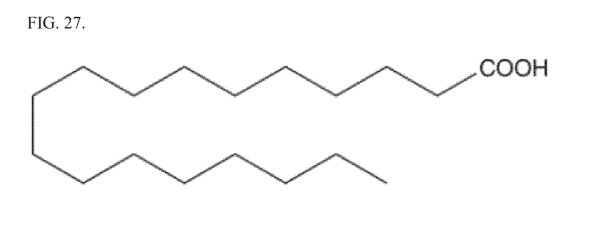

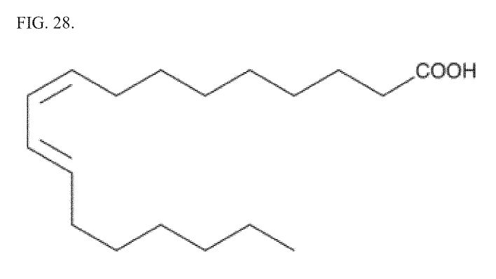

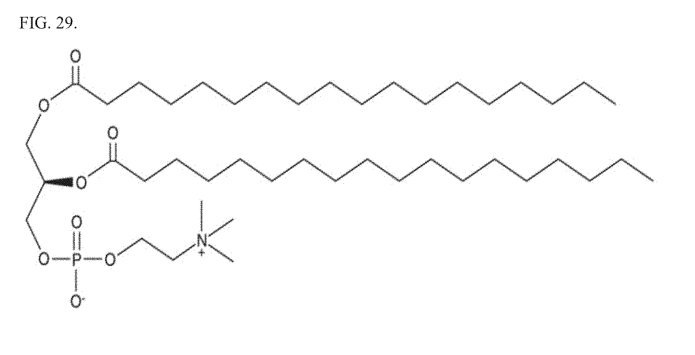

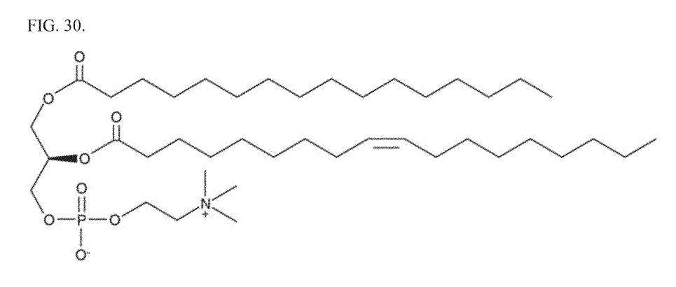

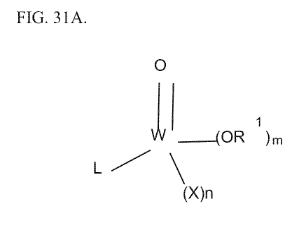

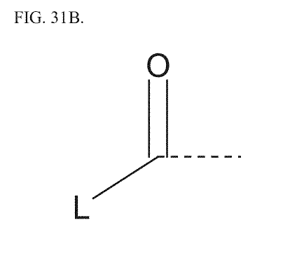

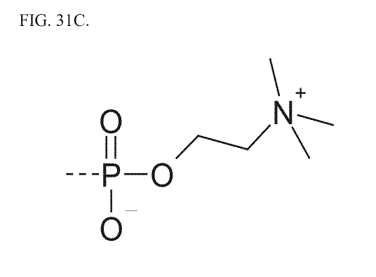

In rice, the closest homolog to the maize pPLAIIα is Os03g27610, a rice patatin-like phospholipase (OspPLAIIφ) with a similar annotation, gene structure and expression pattern, i.e., expressed in pollen and absent elsewhere (FIG. 10). SEQ ID NO: 21 comprises the genomic DNA sequence of Os03g27610, SEQ ID NO: 22 comprises the cDNA sequence, and SEQ ID NO: 23 comprises the amino acid sequence. The close agreement of these features, along with the short evolutionary distance between these two grasses, suggests that a mutation in the rice gene may also give rise to a haploid induction line. In a recent publication the rice protein was detected in sperm nuclei of pollen grains (Abiko et al., 2013), suggesting involvement of this protein in fertilization and/or zygote development.

To improve the haploid induction rate in maize and create the first haploid inducer lines in rice, a reverse genetics TILLING approach was used to obtain novel mutants in the maize GRMZM2G471240 gene and the rice Os03g27610 gene. See McCallum C M et al. (2000) Targeting induced local lesions IN genomes (TILLING) for plant functional genomics, PLANT PHYSIOL. 123: 439-42, incorporated herein by reference. TILLING provides an unbiased approach to generating new mutants as there is no control by the researcher of where the ethylmethanesulfonate (EMS) mutagen will create new mutations. A diversity and abundance of new alleles were generated and tested for haploid induction rate.

Thirteen different TILLING M3 lines were obtained. See Table 14. The PosGenomic column indicates the nucleotide position of the mutation and the change (e.g., G803A indicates that base pair G at position 803 was changed to an A). The effect is the amino acid change or other protein change that results from the mutation (e.g. A209T indicates that an Alanine at amino acid 209 was changed to a Threonine). The BLOSUM score is a prediction of the strength of the effect the amino acid change will have on the protein's conformation or fold (the more negative, the more severe the effect). The “Type” indicates the type of amino acid change (“NSM” means non-silent mutation; “PSM” means partially silent mutation; “silent” means silent mutation; “splice” means splice site mutation resulting in aberrant splicing; “intron” means mutation is in an intron). Finally the GSOR# is the line ID for the Genetic Stocks-Oryza collection at the USDA.

These thirteen lines were selfed to make the M4 and the M4 seed are grown and tested for homozygosity. Homozygous mutant individuals are selfed and outcrossed to test for haploid induction capacity. The resulting progeny are examined for DNA content per cell (ploidy) using the ploidy analyzer.

The non-conservative changes, such as the splice site changes and the changes with most negative BLOSUM scores have the greatest haploid induction potential. These should have the more destabilizing effects on the protein product, and so are the superior haploid induction TILLING alleles compared to the others, giving rise to more haploids per haploid induction cross and likely resulting in partially compromised seed set. Indeed, we have already started to see that in some of the T4 self-pollinations. The line with the lowest seed set was the splice site mutant G153A, with only 29 seeds being recovered per 12 homozygous mutant M4 plants crossed. The other lines had more than 100 recovered.

TABLE 14
TILLING alleles in rice Os03g27610.

| Gene | PosGen | PosTIL | Effect | BLOSUM | Type | GSOR# |
|---|---|---|---|---|---|---|
| Os03g27610 | G803A | G590A | A209T | 0 | NSM | 406317 |
| Os03g27610 | G761A | G548A | D195N | 1 | NSM | 405490 |
| Os03g27610 | G1163A | G950A | G293E | −2 | PSM | 403403 |
| Os03g27610 | G1189A | G976A | G302R | −2 | PSM | 406250 |
| Os03g27610 | T374C | T161C | intron | NA | intron | 403453 |
| Os03g27610 | G1026A | G813A | K247= | NA | silent | 406338 |
| Os03g27610 | C738T | C525T | P187L | −3 | PSM | 405205 |
| Os03g27610 | G1149A | G936A | Q288= | NA | silent | 405898 |
| Os03g27610 | G366A | G153A | splice | NA | splice | 403878 |
| Os03g27610 | G366A | G153A | splice | NA | splice | 405549 |
| Os03g27610 | C792T | C579T | T205I | −1 | PSM | 404794 |
| Os03g27610 | A1021G | A808G | T246A | 0 | NSM | 404534 |
| Os03g27610 | G558A | G345A | V156M | 1 | NSM | 404675 |

Alternately, the rice phospholipase gene found in Os03g27610 may be edited by CRISPR/Cas9 methods. As stated above, there are three key components to the CRISPR process. The first key component is the target sequence. The second is the Cas9, which is the endonuclease. The third key component is the guide RNA (“gRNA”), which is complementary to the target sequence and is responsible for recruiting Cas9 to the desired location. Guide RNAs can be in the form of single guide RNA (sgRNA) or double guide RNA (dgRNA). For rice, we created four constructs targeting the rice phospholipase gene. SEQ ID NO: 38 comprises an expression cassette that provides for dgRNA targeting Os03g27610, in exon 4 very near to where the native four base pair mutation is located in the maize homolog. In the rice gene, the guide RNA target site is GAGACCGGCAGGTACGTCGAGG. SEQ ID NO: 39 comprises an expression cassette that provides for sgRNA targeting Os03g27610, exon 4, at the same gRNA target site as is targeted in SEQ ID NO: 38. The frameshift mutations for both SEQ ID NO 38 and 39 are expected to occur where the vertical bar is placed between the G and the T in the sequence CAGGTACG|TCGAGG (at base pair+1150 of the gDNA sequence in the SEQ ID NO 22. Therefore, both of these constructs are expected to generate haploid inducer mutations that are only seven base pairs downstream from where the maize haploid inducer insertion is located. These mutations in most cases will be frame-shifting mutations that induce small insertions or deletions, for instance a deletion of a G or a T at the cut site, or any other similar mutation. SEQ ID NO: 40 comprises an expression cassette that provides for dgRNA targeting Os03g27610. SEQ ID NO: 41 comprises an expression cassette that provides for sgRNA targeting Os03g27610. Both of these harbor guide RNAs that target the sequence CCTCGCCGATTACTTCGACTGCA in Exon 1. This should generate a knockout of the majority of the coding sequence of the gene. The mutation that is generated should occur at the cut site where the vertical bar is placed between the C and the C in the sequence CCTCGC|CGATTAC (at base pair +215 of the cDNA sequence in SEQ ID NO 22). Therefore both construct 40 and 41 are expected to generate a high frequency of plants containing knockout mutations of the gene, which should also lead to high haploid induction rates in rice.

Rice plants are transformed with a transformation construct comprising a sequence selected from the group consisting of SEQ ID Nos: 38-41. Through the CRISPR/Cas9 machinery encoded in the transformation construct, new phospholipase alleles are generated in the transformants, i.e., the T0 rice plants. T0 rice plants, are grown and crossed (i.e., self-pollinated) to create T1 plants. The T1 rice plants are tested for homozygosity at the new phospholipase allele. Homozygous T1 rice plants are crossed with a rice line, and resulting progeny are tested for haploidy using a ploidy analyzer. Haploid embryos containing no detectable T1 DNA are identified and counted, and the HIR is measured. At least one haploid embryo is produced from the cross, and the HIR is elevated. Preferably, the HIR is at least 5%. The at least one haploid embryo is treated with a chromosome doubling agent, for example colchicine, and a doubled-haploid plant is grown therefrom.

1. A method for inducing haploid seed production, the method comprising treating reproductive tissues of a plant with a composition comprising a compound according to formula (I): 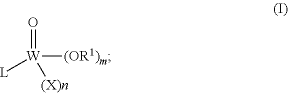
wherein
(a) W is C, P, or S;
(b) m and n are each independently 0 or 1;
(c) X is selected from the group consisting of OH, CN, O(C1-C4 alkyl), halogen, C1-C4 alkyl, and C1-C4 alkyl substituted by one, two, or three halogen or carbonyl;
(d) R1 is:
(i) selected from the group consisting of H, C1-C6 alkyl, and C1-C6 alkyl substituted by one or more hydroxyl groups wherein optionally one or more of said hydroxyl groups is esterified with a radical independently selected from the group consisting of: 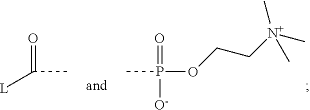
or
(ii) a bond to W when W is S;
(e) each L is independently a C2-C30 carbon chain, said carbon chain optionally comprising one or more groups independently selected from alkenyl, alkynyl, phenyl, and heteroaryl, and said carbon chain optionally interrupted by 1-6 oxygen atoms;
(f) provided that n is 0 when W is C and m is 1; and
(g) provided that n is 1 when W is C and m is 0;
wherein the treatment occurs immediately preceding, during or immediately following pollination, and wherein at least one haploid seed is produced.

2. A method for inducing haploid seed production, the method comprising treating reproductive tissues of a plant with a composition comprising a compound according to formula (I): 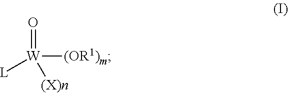
wherein
(a) W is C or P;
(b) m and n are each independently 0 or 1;
(c) X is selected from the group consisting of OH, CN, O(C1-C4 alkyl), halogen, C1-C4 alkyl, and C1-C4 alkyl substituted by one, two, or three halogen or carbonyl;
(d) R1 is selected from the group consisting of H, C1-C4 alkyl, and C1-C4 alkyl substituted by one or more hydroxyl groups;
(e) L is a C2-C30 carbon chain, said carbon chain optionally comprising one or more groups independently selected from alkenyl and alkynyl, and said carbon chain optionally interrupted by 1-6 oxygen atoms;
(f) provided that n is 0 when W is C and m is 1; and
(g) provided that n is 1 when W is C and m is 0;
wherein the treatment occurs immediately preceding, during or immediately following pollination, and wherein at least one haploid seed is produced.

3. The method of claim 1, wherein L of formula (I) is selected from the group consisting of:
(a) (CH2)8-(CH)2-CH2-(CH)2-CH2-(CH)2-CH2-CH3;
(b) (CH2)3-(CH)2-CH2-(CH)2-CH2-(CH)2-CH2-(CH)2-(CH2)4-CH3;
(c) (CH2)7-(CH)2-(CH2)7-CH3;
(d) (CH2)8-(CH)2-CH2-phenyl-CH2-(CH)2-CH2-CH3;
(e) (CH2)8-(CH)2-(CH2)2-O—CH2-(CH)2-CH2-CH3; and
(f) (CH2)8-(CH)2-CH2-phenyl-O—(CH2)3-CH3.

4. (canceled)

5. A method for inducing haploid seed production, the method comprising treating reproductive tissues of a plant with a composition comprising a lipid or a phospholipase inhibitor, wherein the treatment occurs immediately preceding, during or immediately following pollination, and wherein at least one haploid seed is produced.

6. The method of claim 5, wherein the haploid seeds are induced at an increased rate.

7. The method of claim 5, wherein the lipid or the phospholipase inhibitor is selected from the group consisting of compounds listed in Table 7.

8. The method of claim 7, wherein the lipid or the phospholipase inhibitor is selected from the group consisting of methyl alpha-linolenoyl fluorophosphonate (“MALFP”), linoleic acid ethyl ester (“LLAEE”), linoleic acid (“LLA”), corn oil, distearyl-phosphatidyl choline (“DSPC”), or methyl arachidonyl fluorophosphonate (“MAFP”).

9. The method of claim 5, wherein the composition of a lipid or a phospholipase inhibitor comprises a compound selected from Table 7 dissolved or emulsified in a suitable formuation at a concentration of between 0.0001 mg/mL to 100 mg/mL, or between 0.1 mg/mL to 50 mg/mL, or between 1 mg/mL to 10 mg/mL, or is approximately 5 mg/mL.

10. The method of claim 9, wherein the suitable formulation is selected from the group consisting of water, alcohol, DMSO, Tris-EDTA, EDTA, acetic acid, benzene, DME, glycerin, hexane, ethanol, isopropanol, propanol, buffered saline, glycine-HCl, sodium acetate, cacodylate buffer, citrate buffer, So/rensen's phosphate buffer, and phosphate-citrate buffer, barbital buffer, Tris buffer, Glycine-NaOH buffer, Formulation 91, Formulation 92, phosphate buffered saline (“PBS”), PBS plus Tween and DMSO, and any other available solvent or buffer in which a fatty acid may be diluted or emulsified.

11. The method of claim 10, wherein the suitable compound is phosphate buffered saline (“PBS”).

12. The method of claim 5, wherein the plant is a dicot.

13. The method of claim 5, wherein the plant is a monocot.

14. The method of claim 13, wherein the plant is rice.

15. The method of claim 13, wherein the plant is maize.

16. The method of claim 12, wherein the dicot is sunflower or soybean.

17. The method of claim 5, wherein the lipid is applied by a technique selected from the group consisting of: dipping, injection, spray-based topical application, nebulizer, pipette-based topical application, and brush-based topical application, and any other topical application.

18-43. (canceled)
